(19) United States
(12) Patent Application Publication
(10) Pub. No.: US 2026/0102151 A1
(43) Pub. Date: Apr. 16, 2026

(54) KNOTLESS SUTURE ANCHORS

(71) Applicant: S. Wendell Holmes, JR., Columbia, SC (US)

(72) Inventor: S. Wendell Holmes, Jr., Columbia, SC (US)

(21) Appl. No.: 19/359,361

(22) Filed: Oct. 15, 2025

Related U.S. Application Data
(60) Provisional application No. 63/707,395, filed on Oct. 15, 2024.

(51) Int. Cl. A61B 17/04

(52) U.S. Cl. CPC A61B 17/0401; A61B 2017/0414; A61B 2017/044; A61B 2017/0445; A61B 2017/0448

(57) ABSTRACT

A readily implantable knotless suture anchor that de-couples the angle of insertion of the anchor from the tissue passage step and other steps of conventional procedures. An anchor provides an internal locking mechanism configured such that: suture knots do not have to be tied; suture and tissue tension can be easily adjusted intraoperatively; and suture passage and tensioning can be done at a variety of positions and angles. A method includes: passing a first suture through a patient's tissue and then through a loop of a shuttle suture; pulling the open end of the shuttle suture until the first suture passes through an internal suture locking mechanism; applying tension in the first suture until adequate tension is achieved with respect to the tissue; automatically engaging an internal locking member; and securing the first suture against further relative movement with respect to the anchor.

### CROSS-REFERENCE TO RELATED APPLICATION

The present application claims priority to U.S. Provisional Ser. No. 63/707,395, filed Oct. 15, 2025, the disclosure of which is herein incorporated by reference in its entirety.

### FIELD

The present technology is generally related to orthopedic implants broadly related to bone anchors, suture devices, and corresponding surgical tools for driving of an anchor or implant and facilitating the holding and tying of corresponding sutures.

### BACKGROUND

In arthroscopic orthopedic surgery, it is often necessary to reattach soft tissue to a boney structure. This occurs frequently in the shoulder and other tissues. In these situations where soft tissue is being reattached to bone, it is useful to use a suture anchor device to make the necessary repair. A suture anchor is typically placed into the bone and will not ordinarily pull out. Such suture anchor typically has sutures of its own that can be used to effectively reattach and tie down soft tissue to the bone. This reattachment allows the soft tissue to eventually heal to the bone, and during the process of healing, the suture anchor holds the tissue stationary on the bone.

A suture anchor preferably resists gapping at the tissue bone interface and also resists cyclic loosening of the suture and the tissue from the bone. Anchors can be made of metal, plastic, or absorbable plastics, and designs vary with respect to the number of sutures provided and with respect to whether or not such sutures are allowed to slide within the anchor.

In the normal use of suture anchors, the sutures are passed through the tissue and then knots are tied which allow the anchor to be held into place. A disadvantage can be that suture anchors are normally used for an arthroscopic situation where the anchor is being implanted into a joint where access is limited, and also, where fiber optic arthroscopic cameras are required for visualization by the surgeon. Tying knots in such circumstances can be laborious and time-consuming.

Knotless anchors have been introduced in the surgical device market. However, such anchors may have certain technical pitfalls which the present invention seeks to remedy.

The knotless anchors that have been produced to this point typically involve arthroscopically drilling a hole in a bone using a guide, removing the guide from that area, passing sutures through soft tissue. The surgeon must then re-find the hole and insert some sort of push-in anchor or screw-in anchor. As the anchor is screwed or pushed in, it varies as to how much tension is actually developed in the soft tissue. Certain joint geometries and locations make using these types of anchors difficult and/or less effective than desired. The process of drilling the hole and then having to re-find it after removing a specific guide (once the sutures have been passed through the tissue) is a technical difficulty for certain repair locations that have limited access. Additionally, using these types of anchors in certain locations can cause damage to adjacent tissue, which is undesirable.

Conventional suture anchors also only allow a single suture to be attached to adjacent patient tissue. In the case of multiple sutures being required for a particular surgery, a surgeon must install multiple bone anchors.

Accordingly, an anchor design which eliminates the need to tie knots, and which provides internal cinching mechanisms would be desirable. Similarly, an anchor design which allows the tying of multiple sutures to adjacent patient tissue would be desirable.

The above information disclosed in this Background section is only for enhancement of understanding of the background of the present disclosure, and therefore it may contain information that does not form the related art that is already known to one having ordinary skill in the art.

### SUMMARY

The techniques of this disclosure generally relate to bone anchors and suture anchors that may be tightened and maintained at tension without requiring the surgeon to tie a knot.

In one aspect, the present disclosure provides for a two-part suture anchor substantially as described herein.

In one aspect, the present disclosure provides for a suture anchor including a unidirectional woven or nonwoven flexible sleeve for maintaining tension on a suture.

In one aspect, the present disclosure provides for a suture anchor having a plurality of working sutures and at least one shuttle suture.

In one aspect, the present disclosure provides for a flexible suture shell including a unidirectional woven or nonwoven sleeve for maintaining tension on a suture.

In one aspect, the present disclosure provides for a system including any one of the suture anchors disclosed herein and a corresponding driver having a hollow shaft in which a working suture and a shuttle suture may extend therethrough. In various embodiments, a handle of the driver includes a first spool for supporting a free end of the working suture and a second spool for supporting a free end of the shuttle suture.

Generally, the present invention includes, in one embodiment, an easily implantable suture anchor that is knotless, and that de-couples the angle of insertion of the anchor from the tissue passage step and also other steps of the conventional procedure. The present invention anchor provides an internal locking mechanism. i.e., at least one unidirectional suture locking mechanism, configured such that: suture knots do not have to be tied; suture and tissue tension can be easily adjusted intraoperatively; and suture passage and tensioning can be done at a variety of positions and angles.

In one exemplary use of the present invention, a surgeon would pass a first suture through the patient's tissue and then through a loop of a shuttle suture. The free end of a shuttle suture would then be pulled until the first suture next passes through an internal suture locking mechanism constructed in accordance with the present invention. Tension would be applied in the first suture until adequate tension is achieved with respect to the tissue, and then the internal locking member automatically engages and secures the first suture against further relative movement with respect to the anchor.

In another embodiment of an internal locking mechanism constructed in accordance with the present invention, the desired tension is applied in the first suture with respect to the tissue, and then the other end of a line or suture, i.e., a “locking limb,” is pulled in the opposite direction until the internal locking mechanism locks the first suture in place.

In either case, excess suture extending outwardly from the tissue is then cut and removed.

In another exemplary implementation of the present invention, an anchor for implantation in tissue and for use in cooperation with a suture is provided and includes a body member and a cleat connected to the body member that moves between a locking position and an unlocking position. The cleat engages the suture upon being in the locking position and generally fixes movement of the suture with respect to the body member. The cleat substantially permits relative movement between the suture and the body member upon being in the unlocking position, and the cleat automatically moves from the unlocking position to the locking position upon the suture being drawn in a predetermined direction, wherein the anchor is secured to the tissue.

In another implementation, the apparatus further comprises a plurality of teeth being provided on the cleat for engaging the suture upon the cleat being in the locking position.

A further exemplary implementation includes an anchor for implantation in tissue and for use in cooperation with a suture, comprising a body member and a snap lock mechanism connected to the body member that moves between a locking position and an unlocking position. The snap lock mechanism engages the suture upon being in the locking position and generally fixes movement of the suture with respect to the body member. Additionally, the snap lock mechanism substantially permits relative movement between the suture and the body member upon being in the unlocking position, and a line is connected to the snap lock mechanism that moves the snap lock mechanism from the unlocking position to the locking position upon the line being drawn in a predetermined direction, wherein the anchor is secured to the tissue.

In another implementation of the present invention the snap lock device is conically shaped.

The present invention also includes in an exemplary implementation a method for implantation of a suture anchor in tissue, comprising: providing a first suture; providing a shuttle suture; providing an internal suture locking mechanism having a suture lock; passing the first suture through the tissue and then through a loop of the shuttle suture; pulling the free end of a shuttle suture until the first suture next passes through the internal suture locking mechanism; applying tension in the first suture until desired tension is achieved with respect to the tissue; and allowing the suture lock to automatically engage and secure the first suture against relative movement with respect to the anchor.

In one exemplary implementation of the present disclosure, an anchor for implantation in tissue includes a body portion, or shell, defining a passage and a first suture in the passage attached to the shell. A longitudinally extending suture entwinement is provided in the passage, and a shuttle suture having a first end and a second end is provided, where the first end is configured to engage the first suture. The shuttle suture passes in a first direction in the at least one passage and loops to pass in a second direction generally opposite to the first direction into and through the longitudinally extending suture entwinement, where, upon the first suture being received by the first end of the shuttle suture and the second end of the shuttle suture being pulled, the first suture first passes into the longitudinally extending suture entwinement, and the longitudinally extending suture entwinement locks the first suture to the anchor.

Further, a method of the present invention may further comprise applying tension in the first suture with respect to the tissue from one end of the first suture, and through use of the other end of the first suture, pulling in the opposite direction until the suture lock locks the first suture against relative movement with respect to the anchor.

In another exemplary implementation, the anchor may further include a first passage and a second passage in the shell, with the longitudinally extending suture entwinement being proximate to the second passage, where the shuttle suture passing in a first direction in the at least one passage incudes the shuttle suture passing in the first passage and looping to pass into the second passage in the second direction into and through the longitudinally extending suture entwinement.

Other exemplary implementations of the present disclosure include the longitudinally extending suture entwinement being generally a double helix formation comprising the shuttle suture and a first portion of the first suture and/or an entwinement suture, cord, wire, strip, or the like, and configured to allow the shuttle suture to move longitudinally in only one direction with respect the first portion of the first suture and/or the longitudinally extending suture entwinement may comprise the shuttle suture and a portion of the first suture or entwinement suture being generally interlaced or coiled with one another to accomplish a similar result.

In certain exemplary implementations, a partition defines the first passage and the second passage, and the partition defines a partition passage and a post, where the shuttle suture passes through the partition passage when the shuttle suture passes from the first passage to the second passage and where the first suture is tied to the post to attach the first suture to the shell, and may further include the shell defining a lateral passage in communication with at least one of the first passage, the second passage, and the partition passage and where the lateral passage is configured to allow access to the first suture sufficient for attaching, such as by tying, fusing, adhesion, etc., the first suture to the post.

In some exemplary implementations, an anchor is provided for implantation in tissue and includes an elongated shell defining a passage and a partition defining a second passage generally perpendicularly intersecting the first passage and in communication with the exterior of the shell. A first suture is provided in the first passage and attached to the partition, and a unidirectional suture locking mechanism is in the first passage. A shuttle suture is provided having a first end and a second end, where the first end is configured to engage the first suture, and where the shuttle suture passes in a first direction in the passage and looping to pass in a second direction generally opposite to the first direction into and through the unidirectional suture locking mechanism, such that upon the first suture being received by the first end of the shuttle suture and the second end of the shuttle suture being pulled, the first suture passes into the unidirectional suture locking mechanism, and the unidirectional suture locking mechanism locks the first suture to the anchor. In certain implementations, the unidirectional suture locking mechanism is a woven sleeve finger trap, noose and/or a pre-tied knot.

In some implementations, the unidirectional suture locking mechanism is a suture entwinement comprising the shuttle suture and at least one of the first suture, cord, ribbon, strip, strap, or the like, and an entwinement suture.

In certain implementations, the shell includes a first end in communication with the first passage and the second passage and a second end opposite the first end and body member (which can be pivotable) is attached to the second end and defining a suture passage configured for receiving at least one of the first and shuttle sutures.

Other implementations include an anchor for implantation in tissue having a shell defining a passage, a first suture attached to the shell, a unidirectional suture locking mechanism in the passage comprising a plurality of generally opposing teeth configured to receive and engage the first suture and to allow the first suture to move in only one direction with respect to the unidirectional suture locking mechanism. A shuttle suture having a first end and a second end is provided, with the first end being configured to engage the first suture, and the shuttle suture passes in a first direction in the passage and loops to pass in a second direction generally opposite to the first direction into and through the unidirectional suture locking mechanism, where, upon the first suture being received by the first end of the shuttle suture and the second end of the shuttle suture being pulled, the first suture passes into the unidirectional suture locking mechanism, and the unidirectional suture locking mechanism locks the first suture to the anchor. In this implementation, the unidirectional suture locking mechanism may be configured to be insertable into the passage and to automatically couple with the shell upon achieving a predetermined location within the shell.

Further implementations include an anchor for implantation in tissue including an elongated woven or nonwoven flexible or pliable shell, which may be a generally U-shape, and a passage. A first suture passes through the passage the length of the elongated woven or nonwoven pliable flexible shell and has a first end, which may include a loop, and a second end, the first end being configured to engage with the second end (such as by passing through the loop) in a manner that attaches the first suture to the elongated shell. A unidirectional suture locking mechanism is provided in the passage connected to the first suture, and a shuttle suture having a first end and a second end is provided, the first end being configured to engage the first suture, and the shuttle suture passes into and through the unidirectional suture locking mechanism, where, upon the first suture being received by the first end of the shuttle suture and the second end of the shuttle suture being pulled, the first suture passes into the unidirectional suture locking mechanism, and the unidirectional suture locking mechanism locks the first suture to the anchor.

In an implementation, where the elongated shell is generally U-shaped, the shuttle suture passes in a first direction in the at least one passage and loops to pass in a second direction generally opposite to the first direction into and through the unidirectional suture locking mechanism. In some implementations, the unidirectional suture locking mechanisms may include woven or nonwoven flexible/pliable sleeves, elongated suture entwinements, Chinese finger trap type devices (a type of woven sleeves), pre-tied knots, and hangman nooses.

A further implementation may include providing a system including one or more of the anchors disclosed herein and a corresponding driver having a hollow shaft in which a first suture and a shuttle suture may extend therethrough, where a handle of the driver includes a first spool for supporting a free end of the first suture and a second spool for supporting a free end of the shuttle suture.

In an example implementation of the present disclosure, an anchor is provided for implantation in tissue configured for receipt of a suture, with the anchor including a body portion having a bottom portion and defining a first opening in the bottom portion configured for receipt of the suture. The body portion defines a passage in communication with the first opening and which is configured for receiving the suture. At least one of the first opening and the passage is progressively narrowed and configured to form a cinching arrangement to induce friction in the suture as the suture is pulled through the cinching arrangement, where the suture may be selectively fixed with respect to the anchor as the suture is pulled through the cinching arrangement.

In certain implementations, the bottom portion of the anchor includes the first opening defining at least one of a conical shape, a bell shape, a dome shape, a box shape, and a pyramidal shape and/or the passage defines at least one of a conical shape, a bell shape, a dome shape, a box shape, and a pyramidal shape.

In another example implementations, the bottom portion of the anchor includes the first opening and the passage together defining at least one of a conical shape, a bell shape, a dome shape, a box shape, and a pyramidal shape. In example implementations, the anchor further comprising the body portion defining second suture openings in communication with the first opening and configured to allow the suture to pass through the body portion.

Another example implementation of the present disclosure includes an anchor configured for receipt of a suture and for implantation in a boney tissue hole, where the anchor includes a pliable body portion constructed of at least one of ultra-high molecular weight polyethylene, nylon, polyester and other suitable polymer material, and the body portion being configured for receipt of the suture and for insertion into the boney tissue hole. The body portion being configured for bunching up or balling up upon tension being applied to the suture, where, upon the body portion being inserted in the boney tissue hole and tension being applied to the suture, the body portion bunches up or balls up to expand outwardly sufficiently to effectively fix the body portion in the hole, and consequently, the suture to the anchor. In certain implementations, the body portion is constructed of a woven material, nonwoven material and/or an extruded or seamed material.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other aspects, features, and advantages, as well as the following detailed description of the embodiments, will be better understood when read in conjunction with the accompanying drawings. However, the present disclosure is not intended to be limited to the details shown in the drawings, and various modifications and structural changes may be made therein without departing from the spirit of the present disclosure and within the scope and range of equivalents of the claims. Like reference numbers and designations in the various drawings generally indicate like elements.

FIGS. 1A-1EA illustrate a first embodiment of a suture anchor utilizing a first knotless mechanism for maintaining tension of a suture.

FIGS. 1F-1G illustrate a unidirectional woven or nonwoven flexible/pliable sleeve that functions as a suture trap type mechanism for maintaining tension of a suture.

FIGS. 2A-2EA illustrate a second embodiment of a suture anchor utilizing a second knotless mechanism for maintaining tension of a suture.

FIGS. 3A-3F illustrate a third embodiment of a suture anchor utilizing a third knotless mechanism for maintaining tension of a suture.

FIGS. 3G-3GC illustrate an alternate version of the third embodiment of a suture anchor utilizing a third knotless mechanism for maintaining tension of a suture.

FIGS. 4A-4DA illustrate a fourth embodiment of a suture anchor utilizing a two-part anchor and a knotless mechanism for maintaining tension of a suture.

FIGS. 5A-5CB illustrate a surgical tool for use with any of the suture anchor embodiments disclosed herein.

FIG. 6A illustrates a fifth embodiment of a suture anchor with fins utilizing a knotless mechanism for maintaining tension of a suture.

FIG. 6B illustrates a fifth embodiment of a suture anchor with threads utilizing a knotless mechanism for maintaining tension of a suture.

FIGS. 6C-6DA are production drawings of the embodiment of FIG. 6A.

FIGS. 6E-6FA are production drawings of the embodiment of FIG. 6B.

FIGS. 7A-7B illustrate a sixth embodiment of a suture anchor utilizing a knotless mechanism for maintaining tension of a suture.

FIGS. 7C-7DA are production drawings of the embodiment of FIGS. 7A-7B.

FIGS. 8A-8BA illustrates a seventh embodiment of a suture anchor including an insertable suture shell utilizing a knotless mechanism for maintain tension of a suture.

FIG. 9 is a perspective view, with parts cut away, of a knotless suture anchor with an internal suture locking mechanism constructed in accordance with the present invention.

FIG. 10 is a perspective view, with parts cut away, of an alternate embodiment of a knotless suture anchor with an internal suture locking mechanism constructed in accordance with the present invention.

FIG. 11 is a plan view of a knotless suture anchor with an internal suture locking mechanism constructed in accordance with the present invention.

FIG. 12 is a sectional view of an alternate embodiment of a knotless suture anchor with an internal suture locking mechanism constructed in accordance with the present invention.

FIGS. 13-13F are views of an eighth embodiment, and variations thereof, of a hard knotless suture anchor with an internal suture locking mechanism constructed in accordance with the present invention.

FIGS. 14-14B are views illustrating the structure and a first exemplary method of using the eighth embodiment of a knotless suture anchor shown in FIGS. 13-13D in accordance with the present invention, where FIG. 14 is a sectional view of such anchor along Fines 14-14 of FIG. 14B.

FIGS. 15-15B are views illustrating an exemplary method of using the eighth embodiment of a knotless suture shown in FIGS. 13-13F in accordance with the present invention, where FIG. 15 is a sectional view of such anchor along lines 15-15 of FIG. 15B.

FIGS. 16-16B are views illustrating another exemplary method of using the eighth embodiment of a knotless suture shown in FIGS. 13-13F in accordance with the present invention, where FIG. 16 is a sectional view of such anchor along lines 15-15 of FIG. 16B.

FIGS. 17 and 18 are schematic views illustrating further methods of use of the eighth embodiment of a knotless suture shown in FIGS. 13-13F in accordance with the present invention.

FIGS. 19-19B are views illustrating an exemplary method of using the eighth embodiment of a knotless suture shown in FIGS. 13-13F in accordance with the present invention, where FIG. 19 is a sectional view of such anchor along lines 19-19 of FIG. 19B.

FIGS. 20-26 are schematic views of an embodiment of a soft knotless suture anchor with a suture locking mechanism constructed in accordance with the present invention, and exemplary methods of use thereof.

FIGS. 27-27B are views illustrating an exemplary method of using the eighth embodiment of a knotless suture shown in FIGS. 13-13F in accordance with the present invention, where FIG. 27 is a sectional view of such anchor along lines 27-27 of FIG. 27B.

FIG. 28-28A are schematic views of exemplary implementations of a soft knotless suture anchor with a suture locking mechanism constructed in accordance with the present invention, and an exemplary method of use thereof.

FIG. 29 is a schematic view of an of an implementation of suture anchors disclosed herein for a double row tissue repair.

FIG. 30 is a schematic view of an embodiment of a soft knotless suture anchor with a suture locking mechanism constructed in accordance with the present invention, and an exemplary method of use thereof.

FIG. 31 is a schematic view of an of an implementation of suture anchors disclosed herein for a double row repair.

FIGS. 32-35 are schematic views of an embodiment of a soft knotless suture anchor with a suture locking mechanism constructed in accordance with the present invention, and exemplary methods of use thereof.

FIGS. 36 and 36A are views of an exemplary embodiment of a suture anchor insertion tool constructed in accordance with the present invention and an exemplary method of use thereof.

FIGS. 37 and 37A are views of exemplary embodiments of components of the suture anchor insertion tool shown in FIGS. 36 and 36A and an exemplary method of use thereof, where FIG. 37A includes a sectional view taken along lines 37-37 of FIG. 37, of an exemplary eyelet used in connection with such tool.

FIGS. 38-38B are views of exemplary embodiments of components of the suture anchor insertion tool constructed in accordance with the present disclosure shown in FIGS. 36 and 36A and an exemplary method of use thereof, where FIG. 38A is a sectional view taken along lines 37-37 of FIG. 38, of an exemplary eyelet used in connection with such tool, and FIG. 38B illustrates schematically use of such tool in an exemplary insertion of a suture anchor, which could be without limitation a suture anchor as disclosed herein.

FIG. 39 illustrates an exemplary implementation of a soft anchor insertion tool constructed in accordance with the present disclosure in use with a meniscal repair.

FIG. 40 is a schematic view of an exemplary implementation of pre-tied knot constructed in accordance with the present disclosure designed for use in connection with cinchable suture anchors, which could, without limitation, be suture anchors such as disclosed herein.

FIG. 41 is a schematic view of another exemplary implementation of pre-tied knot constructed in accordance with the present disclosure designed for use in connection with cinchable suture anchors, which could, without limitation, be suture anchors such as disclosed herein.

FIGS. 42 and 43 illustrate an exemplary implementation of an insertion tool constructed in accordance with the present disclosure, which, in one application, could be used, without limitation, for meniscal repair with cinchable suture anchors, including suture anchors such as disclosed herein (as shown in FIG. 42).

FIG. 44 illustrates another exemplary implementation of an insertion tool constructed in accordance with the present disclosure, which, in one application, could be used, without limitation, for meniscal repair with cinchable suture anchors, including suture anchors such as disclosed herein.

FIGS. 45, 45A, and 45B illustrate components of a further exemplary implementation of an insertion tool constructed in accordance with the present disclosure, which, in one application, could be used, without limitation, for meniscal repair with cinchable suture anchors, including suture anchors such as disclosed herein.

### DETAILED DESCRIPTION

Embodiments of the present disclosure relate generally, for example, to orthopedic implants, bone anchors, suture devices, and corresponding surgical tools for use in an orthopedic surgery. For example, a surgery in which soft tissue of a patient, such as muscle tissue, is tied off to a bone anchor by way of a suture. Embodiments of the devices and corresponding methods of use are described below with reference to the Figures.

The following discussion omits or only briefly describes certain components, features and functionality related to medical implants, installation tools, and associated surgical techniques, which are apparent to those of ordinary skill in the art of orthopedic surgery and more particularly to the installation of suture devices. It is noted that various embodiments are described in detail with reference to the drawings, in which like reference numerals, when viewed in context, represent like parts, assemblies, and views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may also be used herein to emphasize this meaning and if a value and/or a range of values is provided in the specification or claims with the modifier “about” a meaning of +/− ten percent (10%) to those provided values are encompassed by the meaning of “about,” unless the context clearly indicates otherwise. Further, as used herein, “anchor”and “shell”may at times be used interchangeably.

Components of the disclosed embodiments can be fabricated from biologically acceptable materials suitable for medical applications, including metals, synthetic polymers, ceramics, and bone material and/or their composites. For example, the components, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, and their combinations.

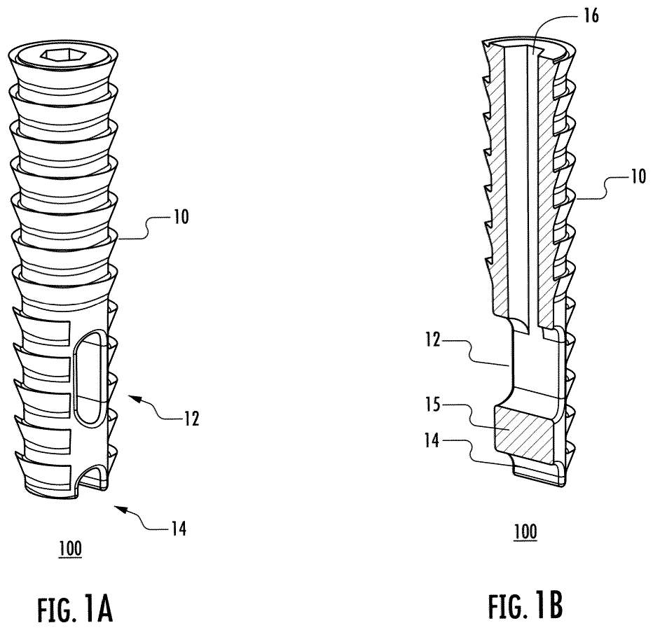

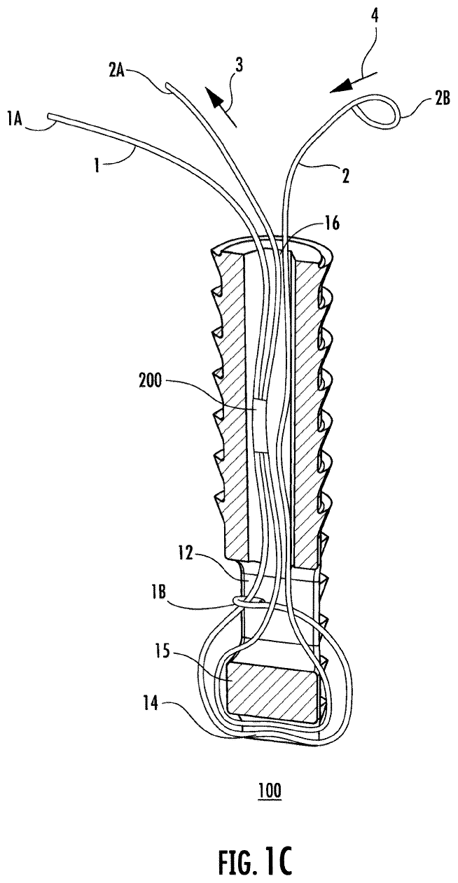

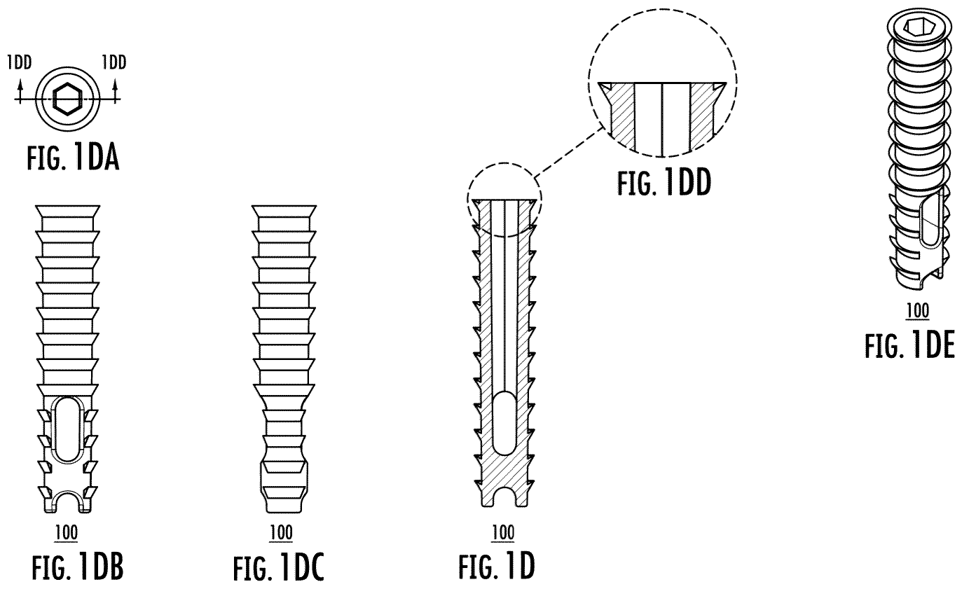

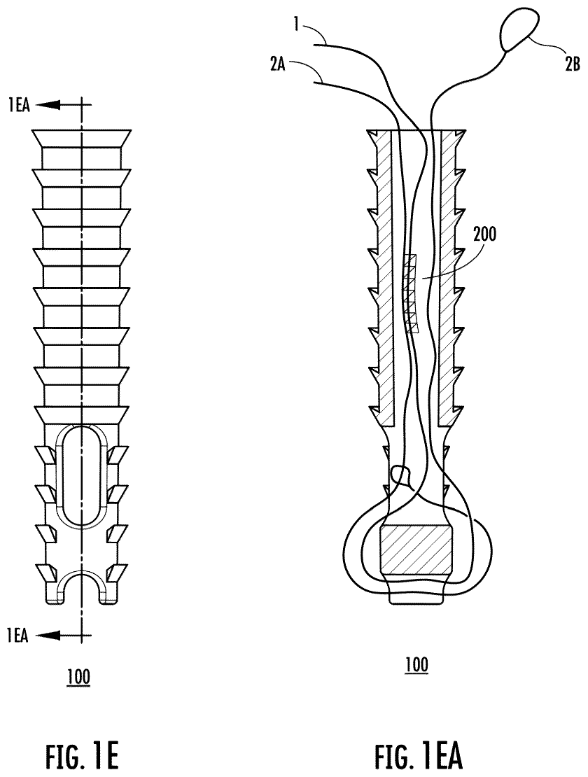

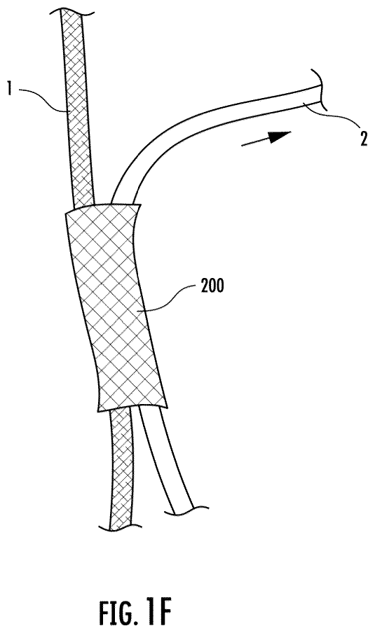

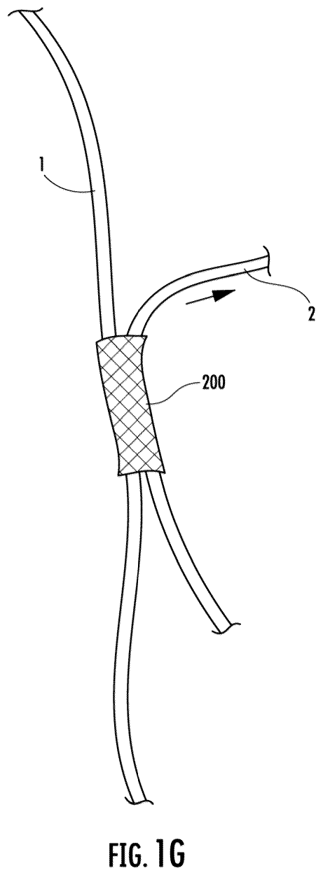

FIG. 1A-FIG. 8B illustrate various embodiments of surgical tools and a suture anchors (i.e., a bone anchor adapted for use with a suture) and various types of knotless weaves, mechanisms, structures, etc. that capture a working suture to maintain a tension on the suture. Various embodiments may also have the advantage of allowing a surgeon to tighten a suture without requiring the surgeon to tie a knot to maintain tension on the suture. In some embodiments, a working suture and a shuttle suture may be used in a cooperative way such that the working suture is attached to adjacent patient tissue and the shuttle suture pulls the working suture towards a bone anchor to a desired tension. In pulling the working suture the capture means (knotless weaves, mechanisms, structures, etc.) allow the working suture to travel forward in a first direction while preventing the working suture from traveling back in an opposite direction. In this sense, the various suture capture means disclosed herein may be referred to as “unidirectional” or “knotless” in that they automatically maintain a tension on a working suture. More detailed examples of the various ways the objects of this disclosure may be implemented are discussed below with reference to the detailed figures.

FIGS. 1A-1EA illustrate a first embodiment of a suture anchor 100 utilizing a first knotless mechanism for maintaining tension of a suture. Referring to FIGS. 1A-1B, it is shown that a suture anchor includes a plurality of ridges 10 sequentially disposed along an outside circumference of the anchor 100. The ridges may facilitate and/or be configured to allow the suture anchor 100 to be tapped into an aperture or hole in boney anatomy. For example, a surgeon may tap the anchor 100 into a pre-existing hole in boney anatomy and the anchor 100 may be held in place by a friction fit. The suture anchor 100 may include a central longitudinal passageway 16 extending from a top of the suture anchor towards a bottom of the suture anchor (i.e., a proximal end for receiving a drive tool to a distal end/leading end that is inserted into boney anatomy). The suture anchor 100 may include a first lateral passageway 12 separated from a second lateral passageway 14 by a block 15 which closes or terminates the longitudinal passageway 16. For example, the first lateral passageway 12 may extend through the side of the suture anchor 100 thereby communicating with the central longitudinal passageway 16. In some embodiments (not illustrated) the second lateral passageway 14 may extend through the side of the suture anchor 100.

FIG. 1C illustrates a working suture 1 having an open end 1A (or free end 1A) and a looped end 1B. The open, or free, end 1A may have a hook or needle to puncture through adjacent tissue (not illustrated) and the looped end 1B may be integrally formed with the working suture 1 during manufacture or tied by hand in advance of performing a surgery. Similarly, a shuttle suture 2 may have an open, or free, end 2A and a looped end 2B. In this embodiment, both ends of the shuttle suture 2 are free, e.g., the shuttle suture is not secured to the bone anchor 100 like the working suture 1 is. As illustrated, the working suture 1 is secured at the looped end 1B to the block portion 15 by passing through the longitudinal passageway 16 and looped around the block structure 15 through the first lateral passageway 12 and second lateral passageway 14. In use, a surgeon may pass a hook or other piercing structure (not illustrated) attached to the open end 1A of the working suture 1 through adjacent patient tissue at least once, although often several times. Next, the surgeon may pull on the open end 1A of the working suture 1 to pull the attached and adjacent patient tissue closer to the anchor 100. Thereafter, the surgeon may loop the open end 1A of the working suture through the looped end 2B of the shuttle suture and/or attach the working suture 1 open end 1A to the shuttle suture 2 looped end 2B. Thereafter, the surgeon may pull the shuttle suture open end 2A upwards (arrow 3) and away from longitudinal passageway 16. In doing so the looped end 2B of the shuttle suture 2 and the open end 1A of the working suture are pulled downward (arrow 4) into the passageway 16 (at least marginally). Given the free end 2A shuttle suture 2 has been preloaded through a unidirectional suture locking mechanism, such as through and internal passage of a unidirectional woven or nonwoven flexible sleeve 200 (see FIGS. 1F-1G), continued pulling of shuttle suture 2 upwardly (in the orientation of suture anchor 100 shown in FIG. 1C) eventually draws both shuttle suture looped end 2B and working suture 1 upwardly through unidirectional sleeve 200, and still further upward pulling of shuttle suture 2 causes shuttle suture 2 to exit sleeve 200, with working suture 1 passing by itself through sleeve 200. It is to be understood that a variety of unidirectional suture locking configurations could be used instead of or in addition to sleeve 200, including without limitation, suture entwinements, Chinese finger trap type devices (a type of woven sleeves), pre-tied knots, and hangman nooses. The internal passage is configured to only allow passage of one or more sutures in a first predetermined direction and attempts to pull one or more of the sutures in a second direction opposing the first direction causes fibers, yarns and/or other elements within the internal passage to engage, grip and restrain the one or more sutures from movement in the second direction.

In so doing, the working suture 1 is allowed to move in one direction only, in that attempting to pull working suture 1 in the other direction would cause a tightening process, whereby unidirectional woven sleeve 200 engages and grips working suture to substantially prevent the pulling of working suture 1 in the other direction. For example, the working suture 1 may only move in one direction due to the unidirectional woven sleeve 200 (see FIGS. 1F-1G) which functions similarly to a Chinese finger trap. In this way, as the surgeon pulls on the shuttle suture 2 the working suture 1 pulls the adjacent tissue closer to the bone anchor 100 and maintains the tension to the working suture by binding or cinching together. As shown in FIGS. 1F-1G, the unidirectional woven sleeve 200 may be integrally woven onto a side portion of working suture 1. In use, as the shuttle suture 2 is pulled upward and away a cinching function (binding function) occurs via friction between the unidirectional sleeve 200 and the working suture, thereby maintaining a tension to the working suture 1. Stated another way, the interior diameter of the woven sleeve may, in effect, decrease and compress against the working suture 1 thereby capturing the working suture 1 in place, i.e., substantially refraining the pulling of the working suture in a direction opposite the direction in which the shuttle suture 2 pulls the working suture 1. The internal passage can be similar to the unidirectional sleeve 200 discussed above, in that it only allows passage of sutures in one predetermined direction and attempts to pull one or more sutures in a second direction opposing such predetermined direction causes fibers, yarns, or other elements within the internal passage to engage, grip and restrain the one or more sutures from movement in the second direction.

FIGS. 1D-1EA are production drawings which show the suture anchor 100 from alternate perspectives, where FIG. 1DA is a plan view, FIG. 1 DB is an elevational view, and FIG. 1DC and 1DD are sectional views of anchor 100 (taken along the arrows shown in FIG. 1DA), FIG. 1DE is a perspective view of anchor 100, and FIG. 1EA is a sectional view taken in the direction of the arrows in FIG. 1E. The particular dimensions shown in these drawings are example in nature only, i.e., relative dimensions may be adjusted.

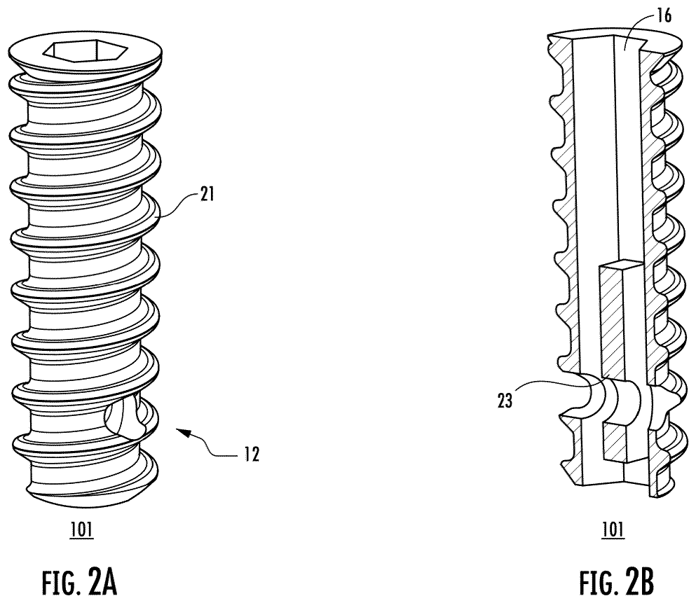

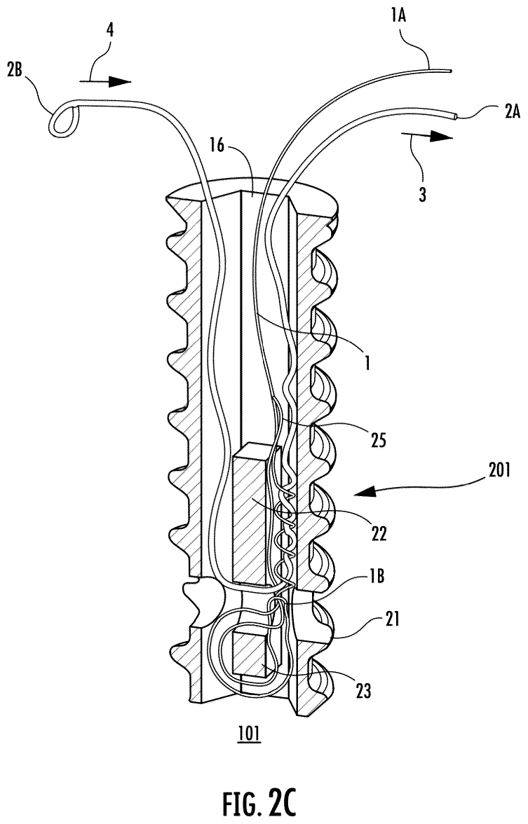

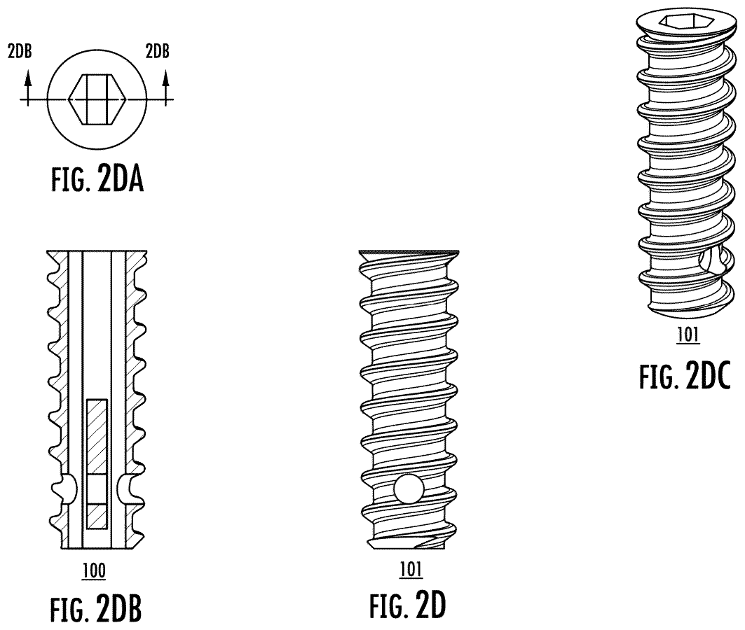

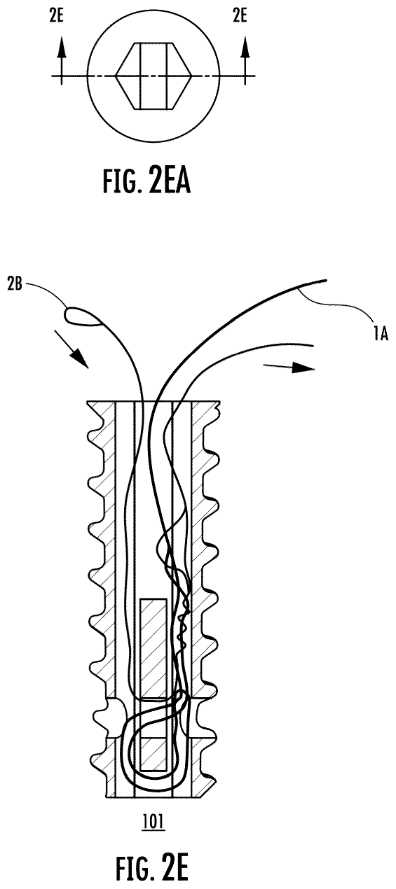

FIGS. 2A-2EA illustrate a second embodiment of a suture anchor 101 utilizing a second knotless mechanism for maintaining tension of a suture. Suture anchor 101 may include the same, similar, or substantially the same features and functionality as explained above with respect to suture anchor 100. Accordingly, duplicative description will be omitted and like numbering of parts will be maintained where possible. As illustrated, anchor 101 may include a thread pattern for rotatably screwing the anchor 101 into boney anatomy of a patient. Anchor 101 may also include a central passageway 16 including a lateral passageway 12 that passes through the central longitudinal passageway 16. A first partition or block 22 may be disposed above the lateral passageway 21 (which may be configured to allow access to the working suture 1 sufficient for tying such suture 1 to the post, or, block 23) and a second block 23 may be disposed beneath the lateral passage or passageway 21. Referring to FIG. 2C, the working suture 1 may be secured to the anchor 101 by being passed down through passageway 16 and anchored to the second block 23 by a double pass looped end 1B. One of the ends of the shuttle suture may extend upwards through the passageway 16 without being woven around the shuttle suture 2 while the other end of the working suture may be coiled around the coils of the shuttle suture to form a unidirectional suture locking mechanism, such as a longitudinally extending suture entwinement which may include a noose like configuration 201, and could also include a knot. As shown in FIGS. 2C and 2E, the unidirectional suture locking mechanism 201 includes working suture 2 and shuttle suture 2 being intertwined, or enmeshed, with one another, but it is to be understood, however, that other unidirectional suture locking configurations could be used, including without limitation, Chinese finger trap type devices, pre-tied knots, and hangman nooses. The number of coils may vary from about 3 to about 9 depending on the type of suture material. In at least one embodiment, the number of coils may range from about 4-5 coils of the working suture 1 around the shuttle suture 2. In some embodiments, at least one or both of the sutures 1, 2 may be circle like or oval like in cross section and in other embodiments at least one or both of the sutures 1, 2 may be ribbon like. In those embodiments in which the working suture 1 is a ribbon like suture, the shuttle suture 2 may optionally pierce the working suture 1 at a piercing location 25. The elongated, or longitudinally extending, suture entwinement 201 may include a double helix formation comprising the shuttle suture 2 and a first portion of the first suture 1 and/or a separate entwinement suture apart from working suture 1, if desired. It is to be understood that as used herein, “entwinement suture” may include use of a suture, cord, string, cable, rope, strand, wire, strap, ribbon, or the like.

Similarly, as explained above with respect to anchor 100, a surgeon may loop the open end 1A of the working suture through the looped end 2B of the shuttle suture and/or attach the working suture 1 open end 1A to the shuttle suture 2 looped end 2B. Thereafter, the surgeon may pull the shuttle suture open end 2A upwards and away (arrow 3) from longitudinal passageway 16. In doing so the looped end 2B of the shuttle suture 2 and the open end 1A of the working suture are pulled downward (arrow 4) into the passageway 16 (at least marginally). Given the free end 2A shuttle suture 2 has been preloaded through the noose like knot 201 (see FIG. 2C), continued pulling of shuttle suture 2 upwardly (in the orientation of suture anchor 101 shown in FIG. 2C) eventually draws both shuttle suture looped end 2B and working suture 1 downwardly and around the bottom end of first block 22 and upwardly through the noose like knot 201, and still further upward pulling of shuttle suture 2 causes shuttle suture 2 to exit the noose like knot 201, with working suture 1 passing by itself through the noose like knot 201. In doing so, the noose like knot 201 will bind and thereby maintain the tension in the working suture 1 in a manner similar in effect as discussed above regarding unidirectional sleeve 200 of the first embodiment of suture anchor 100 discussed above.

FIGS. 2D-2EA are production drawings which show the suture anchor 101 from alternate perspectives, where FIG. 2DA is a plan view, FIG. 2 DB is a sectional view of anchor 101 (taken along the arrows shown in FIG. 2DA); and FIG. 2DC is a perspective view of anchor 101. The particular dimensions shown in these drawings and the corresponding commentary are example in nature only, i.e., relative dimensions may be adjusted.

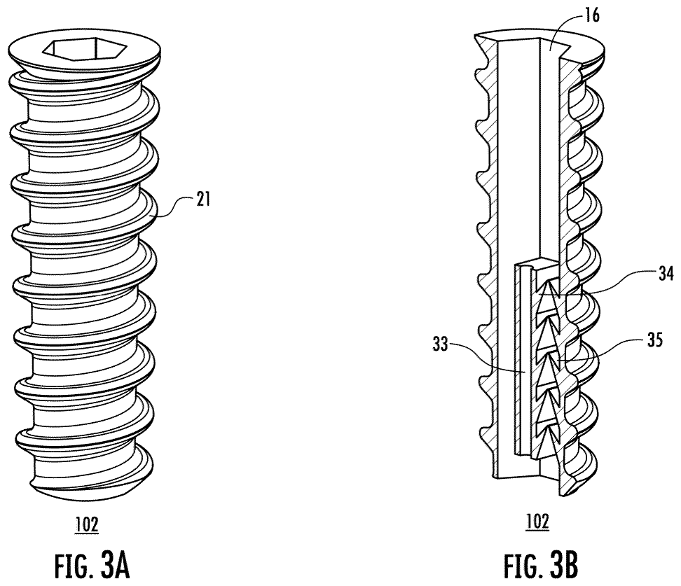

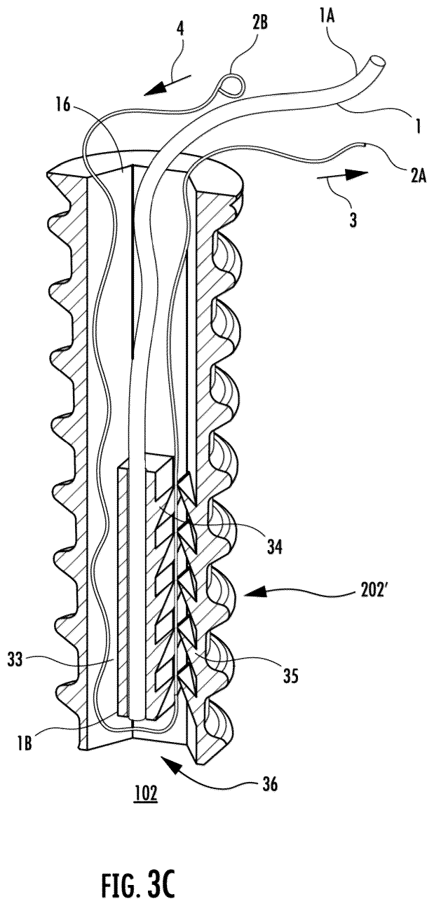

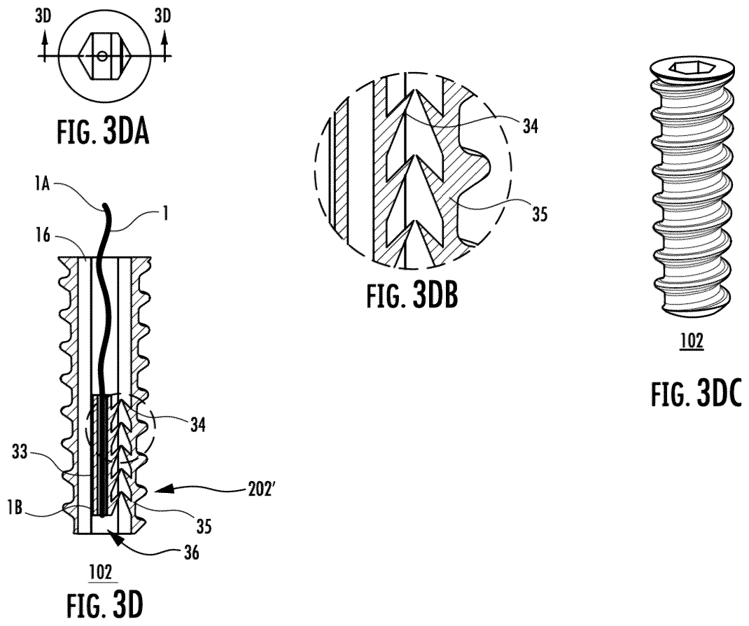

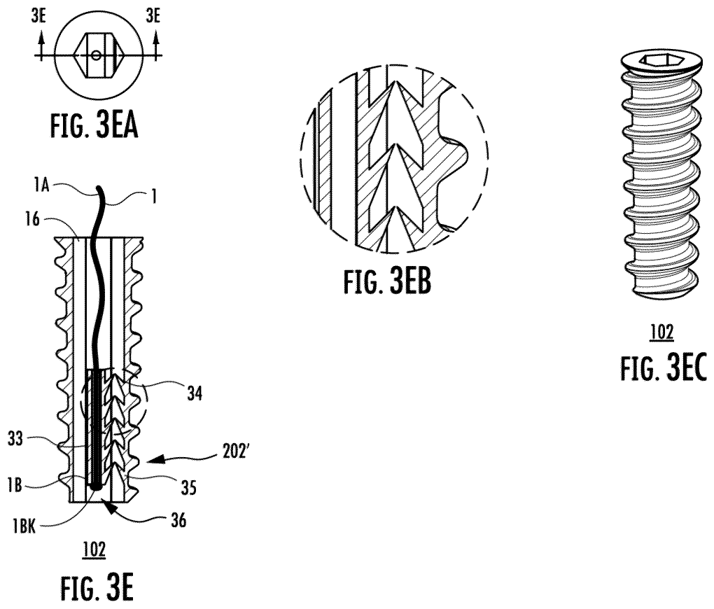

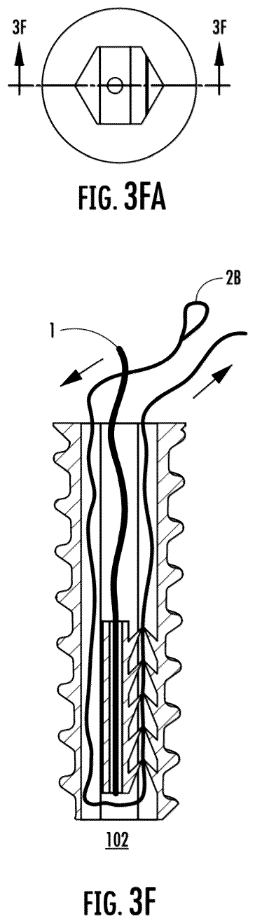

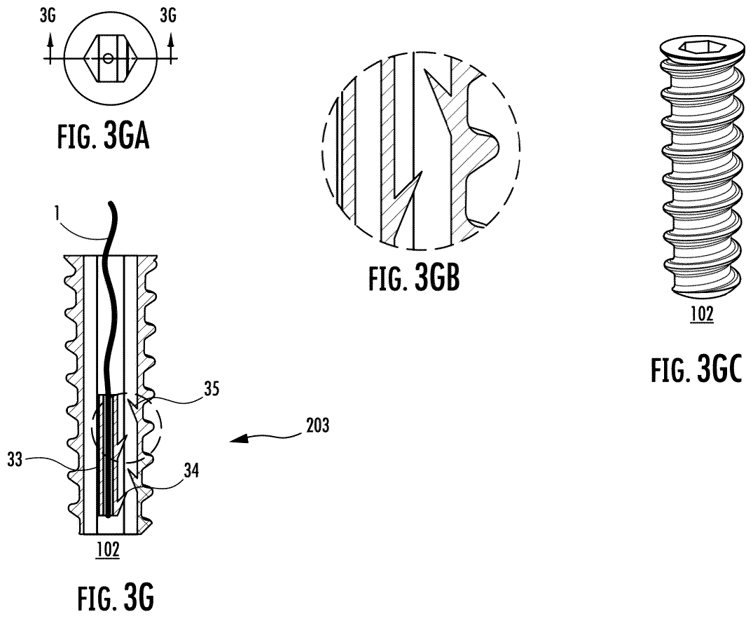

FIGS. 3A-3FA illustrate a third embodiment of a suture anchor 102 utilizing a third knotless mechanism for maintaining tension of a suture, where FIG. 3F is a sectional view of anchor 102 taken in the direction of the arrows in FIG. 3FA. Suture anchor 102 may include the same, similar, or substantially the same features and functionality as explained above with respect to suture anchors 100 and101. Accordingly, duplicative description will be omitted and like numbering of parts will be maintained where possible. As illustrated, anchor 102 may include a thread pattern for rotatably screwing the anchor 102 into boney anatomy of a patient. Consistent with the disclosure and inventive spirit herein thread pattern may be substituted for a tap in style anchoring means, e.g., fins 10 or ribs or barbs. Anchor 102 may also include a central passageway 16 including an anchoring block 33 for securing a working suture 1 thereto. Working suture 1 may be secured to or integrally formed with anchoring block 33, e.g., working suture 1 may be secured by a knot (see FIG. 3E) at a lowermost end thereof or adhered by epoxy or the like. In some embodiments, anchoring block 33 may include a central passageway for passing working suture 1 therethrough. Anchoring block 33 may include at least one tooth like protrusion 34 that extends at an angle away from anchoring block 33 in the longitudinal passageway 16. The tooth like protrusions 34 may extend towards the adjacent tooth like protrusions 35 formed on an interior sidewall of anchor 102. In this way, the generally opposing teeth like protrusions 34, 35 may form a unidirectional suture locking mechanism, namely, a securing structure 202′ that may allow the shuttle suture 2 to be pulled therethrough between the adjacent corresponding teeth in a single direction while binding the shuttle suture 2 and preventing it from being pulled out in the opposing direction.

Referring to FIG. 3C, the working suture 1 may be secured to the anchor 102 via the anchoring block 33 at fixed end 1B. In this way, the working suture 1 may extend out of passageway 16. The shuttle suture 2 may extend down through passageway 16 and underneath the anchoring block 33 and be passed back upwards between the teeth like protrusions 34, 35 of the unidirectional securing structure 202′. Similarly, as explained above with respect to anchors 100, 101 a surgeon may loop the open end 1A of the working suture through the looped end 2B of the shuttle suture and/or attach the working suture 1 open end 1A to the shuttle suture 2 looped end 2B. Thereafter, the surgeon may pull the shuttle suture open end 2A upwards and away (arrow 3) from longitudinal passageway 16. In doing so the looped end 2B of the shuttle suture 2 and the open end 1A of the working suture are pulled downward (arrow 4) into the passageway 16 (at least marginally). Given the free end 2A shuttle suture 2 has been preloaded through the unidirectional securing structure 202′ (see FIG. 3C), continued pulling of shuttle suture 2 upwardly (in the orientation of suture anchor 102 shown in FIG. 3C) eventually draws both shuttle suture looped end 2B and working suture 1 downwardly and around the bottom end of anchoring block 33 and then upwardly through the unidirectional securing structure 202′, and still further upward pulling of shuttle suture 2 causes shuttle suture 2 to exit the unidirectional securing structure 202′, with working suture 1 passing by itself through the unidirectional securing structure 202′. In doing so, the unidirectional securing structure 202′ will bind and thereby maintain the tension in the working suture 1 in a manner similar in effect as discussed above regarding the noose like knot 201 of the second embodiment of suture anchor 101 discussed above. In doing so, the teeth like protrusions will bind the shuttle suture 2 in place and thereby maintain the tension in the working suture 1.

In FIG. 3C, the ratchet teeth like protrusions are disposed directly adjacent to one another on opposing surfaces, e.g., they may be diametrically opposed. In an alternate and similar embodiment shown in FIG. 3G, the ratchet teeth like protrusions 34, 35 are offset from one another. Ratchet teeth like protrusions 34 may be arranged like a helix or spiral winding up and coiled around the corresponding surfaces of the block 33. Similarly, the ratchet teeth like protrusions 35 may be arranged like a helix or spiral winding up and coiled around the corresponding sidewall surface of passageway 16. In this way, the helix pattern of ratchet teeth like protrusions 34, 35 may form a unidirectional securing structure 203 having helically oriented teeth like protrusions. A method of operation of this type of securing structure 203 may be the same or substantially the same as explained above with respect to securing structure 202′.

FIGS. 3D-3FA are production drawings which show the suture anchor 102 from alternate perspectives where FIG. 3DA is a plan view, FIG. 3D is a sectional view of anchor 102 (taken along the arrows shown in FIG. 3DA), FIG. 3DB is a detail view taken from FIG. 3D, and FIG. 3DC is a perspective view of anchor 102. The particular dimensions shown in these drawings and the corresponding commentary are example in nature only, i.e., relative dimensions may be adjusted.

FIGS. 3G-3GC are a production drawings which show an alternate version of the suture anchor 102, where FIG. 3GA is a plan view of anchor 102, FIG. 3G is a sectional view of anchor 102 (taken along the arrows shown in FIG. 3GA), FIG. 3GB is a detail view taken from FIG. 3G, and FIG. 3GC is a perspective view of anchor 102. The particular dimensions shown in these drawings and the corresponding commentary are example in nature only, i.e., relative dimensions may be adjusted. In the version of the anchor 102 shown in FIG. 3G, teeth 34, 35 are alternatingly offset vertically with respect to one another (in the orientation of FIG. 3G) such that shuttle suture 2 and working suture 1 follow essentially a serpentine path through securing structure 203.

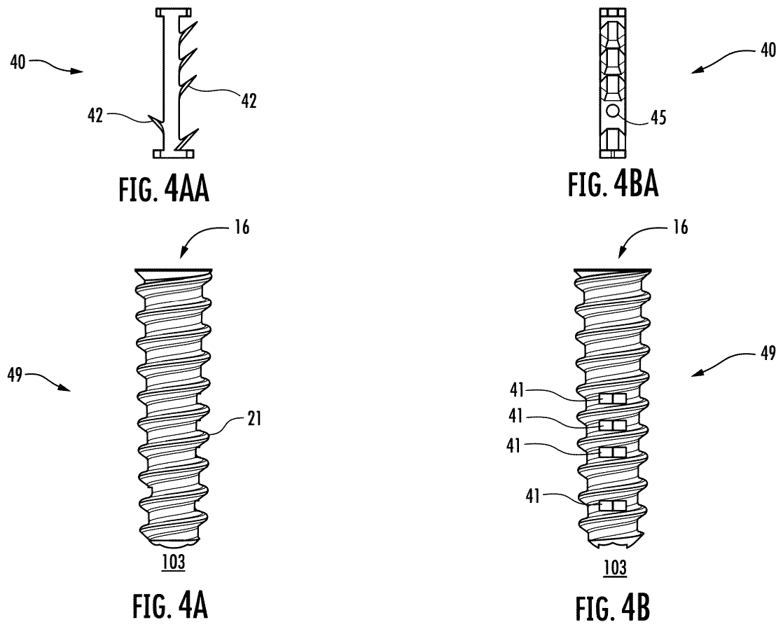

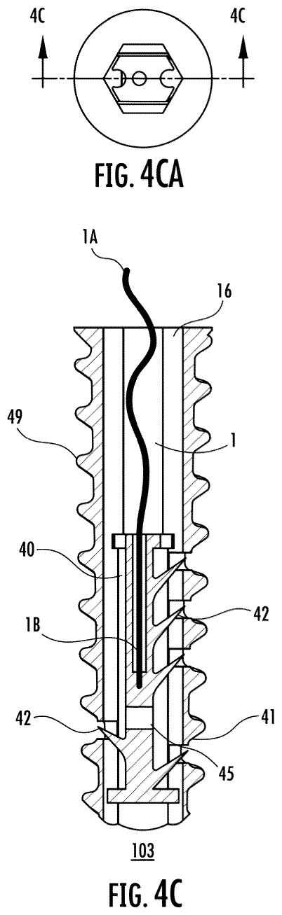

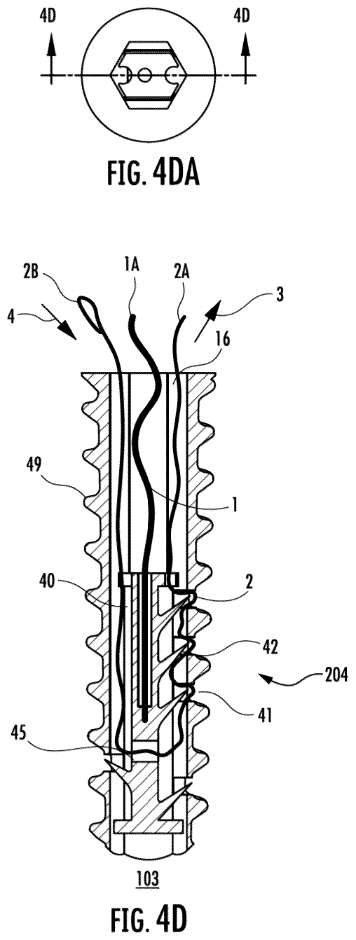

FIGS. 4A-4DA illustrate a fourth embodiment of a suture anchor 103 utilizing a two-part anchor and a unidirectional suture locking mechanism, namely, a knotless mechanism 204 for maintaining tension of a suture. Suture anchor 103 may include the same, similar, or substantially the same features and functionality as explained above with respect to suture anchors 100, 101, 102. Accordingly, duplicative description will be omitted and like numbering of parts will be maintained where possible. Anchor 103 may be a two-part anchoring system in which a first component 49 (FIGS. 4A and 4B) may be configured for anchoring to boney anatomy of a patient and a second component 40 may be disposed within a central passageway 16 of first component 49. As illustrated, first component 49 may include a thread pattern for rotatably screwing the two-part anchor 103 into boney anatomy of a patient. Consistent with the disclosure and inventive spirit herein thread pattern may be substituted for a tap in style anchoring means.

First component 40 may be configured to be inserted inside of a central passageway 16 of second component 49. As shown in FIG. 4C, the first component 40 may include an anchor point for securing a working suture 1 thereto. Working suture 1 may be secured to or integrally formed with the first component 40, e.g., working suture 1 may be secured by a knot, adhered by epoxy, interference fit, thermal or ultrasonic welding, or by some other fixation technique. In some embodiments, first component 40 may include a central passageway for passing working suture 1 therethrough to secure working suture 1 to the first component 40. As shown in FIGS. 4A and 4B, the first component 40 may include at least one tooth like protrusion 42 that extends outward at an angle. When the two-part anchor is disposed within the central passageway 16 of the second component 49, the teeth like protrusions 42 may extend away at an angle of about 30 degrees to 60 degrees from the longitudinal extension direction of the first component 40. In this way, when the first component 40 is positioned inside of the longitudinal passageway 16 the tooth like protrusions 42 (FIG. 4AA and 4BA) may extend upwards and through the windows 41 (aperture or opening) extending through a sidewall of second component 49. Referring to FIGS. 4C-4D, where FIG. 4C is a sectional view of anchor 103 taken along the arrows show in a plan view of anchor 103 in FIG. 4CA, and where the teeth like protrusions 42 of the first component may extend upwards and be seated within the windows 41. First component 40 may also include a passageway 45 for passing of the shuttle suture 2 therethrough as will be explained in further detail below.

As seen best in FIG. 4D, where FIG. 4D is a sectional view of anchor 103 taken along the arrows show in a plan view of anchor 103 in FIG. 4DA, and where the working suture 1 may be secured to the first component 40 at fixed end 1B. In this way, the working suture 1 may extend out of passageway 16. The shuttle suture 2 may extend down through passageway 16 and through shuttle passageway 45 of the first component 40 and be passed back upwards between the teeth like protrusions 42 and windows 41 of the unidirectional securing structure 204. Similarly, as explained above with respect to anchors 100, 101, 102 a surgeon may loop the open end 1A of the working suture through the looped end 2B of the shuttle suture and/or attach the working suture 1 open end 1A to the shuttle suture 2 looped end 2B. Thereafter, the surgeon may pull the shuttle suture open end 2A upwards and away (arrow 3) from longitudinal passageway 16. In doing so the looped end 2B of the shuttle suture 2 and the open end 1A of the working suture are pulled downward (arrow 4) into the passageway 16 (at least marginally). Given the free end 2A shuttle suture 2 has been preloaded through the unidirectional securing structure 204 (see FIG. 4D), continued pulling of shuttle suture 2 upwardly (in the orientation of suture anchor 103 shown in FIG. 4D) eventually draws both shuttle suture looped end 2B and working suture 1 downwardly and around the bottom end of first component 40 and then upwardly through, in serpentine fashion, through the interfaces of teeth 42 and windows 41 of the unidirectional securing structure 204, and still further upward pulling of shuttle suture 2 causes shuttle suture 2 to exit the unidirectional securing structure 204, with working suture 1 passing by itself through the unidirectional securing structure 204. In doing so, the unidirectional securing structure 204 will bind and thereby maintain the tension in the working suture 1 in a manner similar in effect as discussed above regarding the noose like knot 201 of the second embodiment of suture anchor 101 discussed above. In doing so, the teeth like protrusions 42 will unidirectionally bind the working suture 1 in place and thereby maintain the tension in the working suture 1. First component 40 may be configured to be insertable into the passageway 16 and to automatically couple, such as by a snap or interference fit, with the anchor, or shell, upon achieving a predetermined location within the shell.

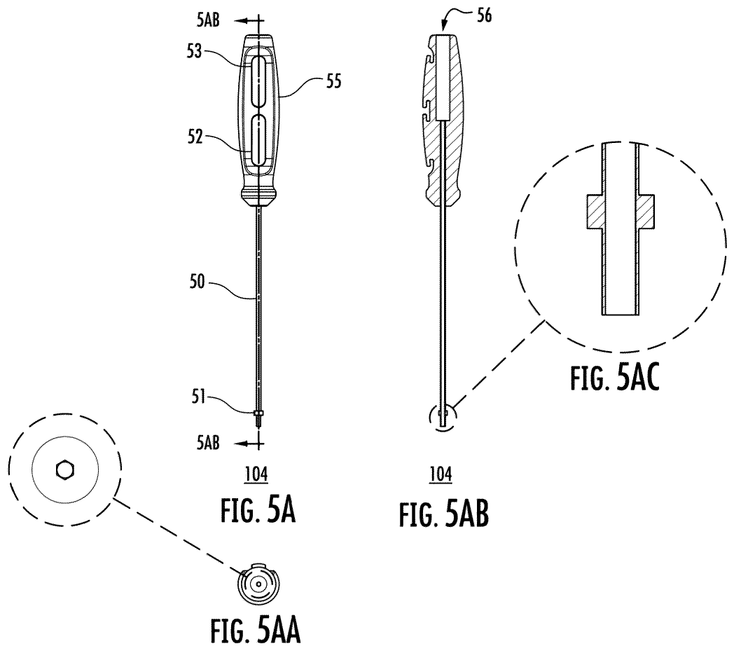

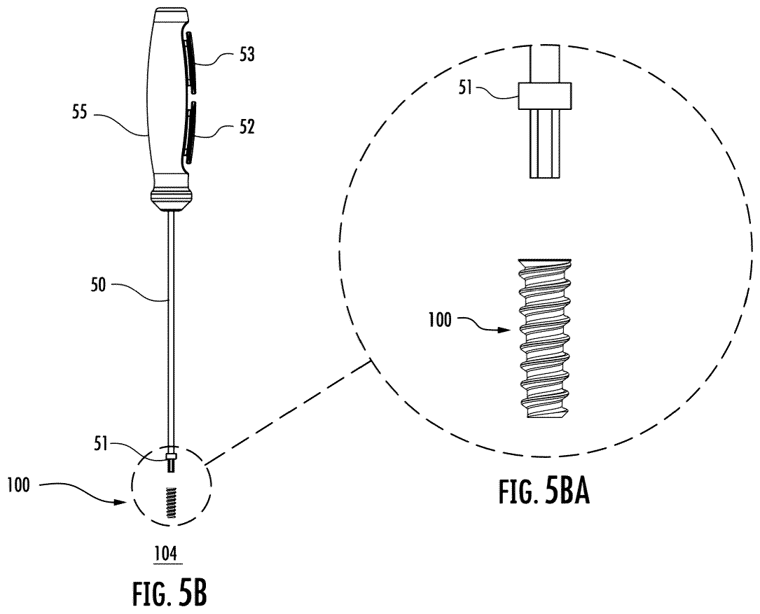

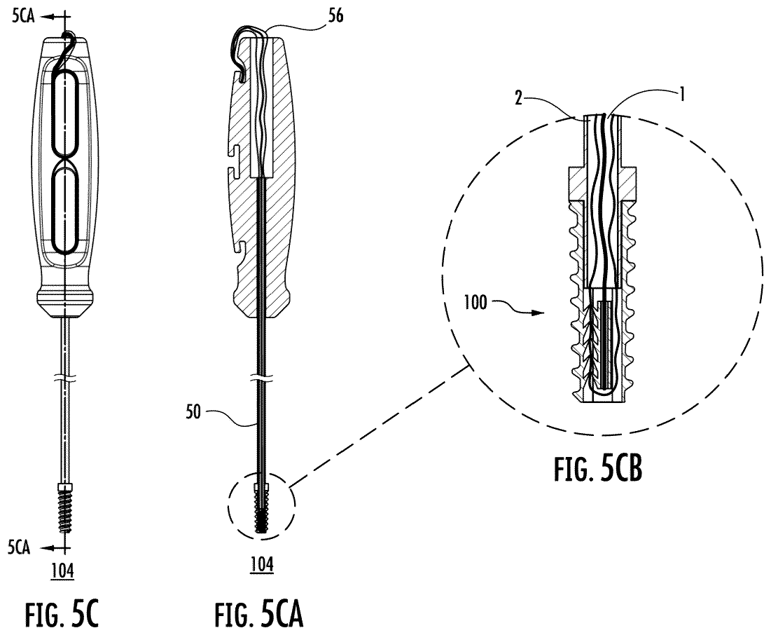

FIGS. 5A-5CB illustrate a surgical tool 104 for use with any of the suture anchor embodiments disclosed herein, where FIG. 5AB and 5CA are sectional views of surgical tool 104 (and FIG. 5AC is a detail view taken from FIG. 5AB, and FIG. 5CB is a detail view taken from FIG. 5CA), and FIG. 5AA is a plan view with a detail of the top of surgical tool 104. The surgical tool 104 may be used for driving any of the various anchor structures 100, 101, 102, 103, etc. disclosed herein. Additionally, the surgical tool 104 may be used as a complete pre-arranged system, kit, sterile pack, etc., in which any of the various anchor structures disclosed herein, including the working suture 1, and shuttle suture 2, are pre-attached and the ends thereof are attached to the outside of the surgical tool 104.

Surgical tool 104 may include a handle 55 attached to a hollow drive shaft 50. An end of the hollow drive shaft 50 may include a shoulder 51 to provide a stop surface against the top of a corresponding suture anchor 100, 101, 102, 103, etc. The hollow drive shaft 50 and handle 55 may also form a central passageway 56 for passing the working suture 1 and shuttle suture 2 therethrough. In this way, the sutures 1, 2 may pass through the hollow shaft 50 and out of the handle 55. Additionally, the outside of the handle 55 may include a first mounting spool 52 for coiling the slack of the working suture 1 and a second mounting spool 53 for coiling the slack of shuttle suture 2. For example, the free ends of the working suture 1 and shuttle suture 2 may be coiled around the corresponding mounting spools 52, 53. As shown in FIG. 5C, the working suture 1 and shuttle suture 2 may extend from the suture anchor 100, up through the hollow shaft 50 of the surgical tool through passageway 56 and then be coiled or spooled around the spools 52, 53.

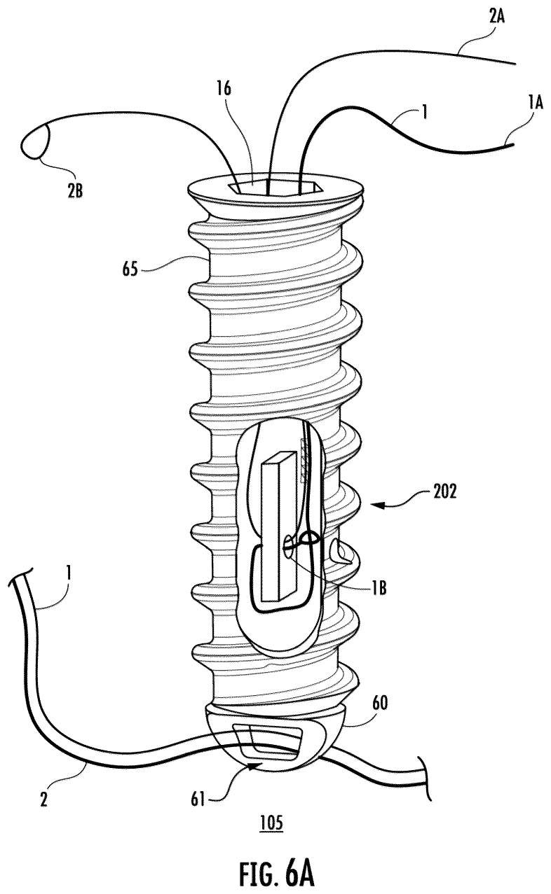

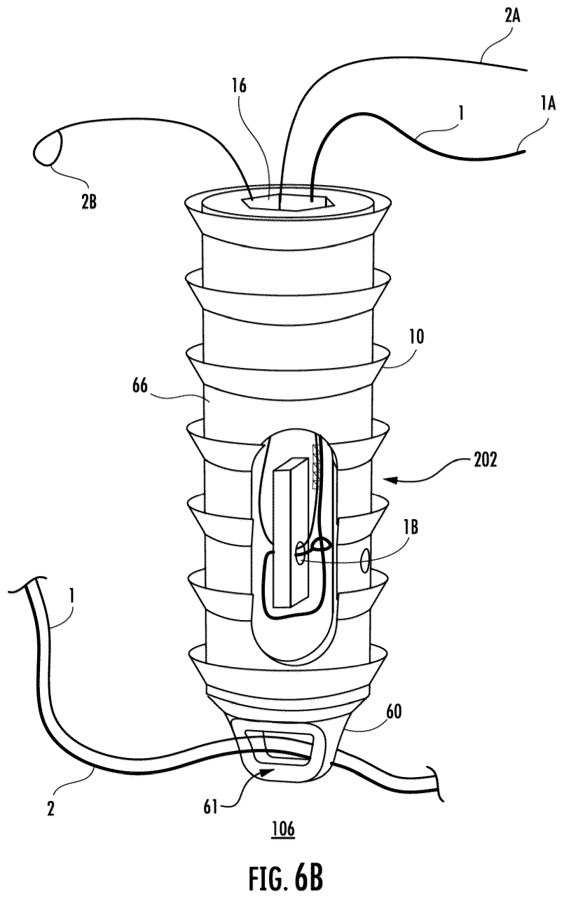

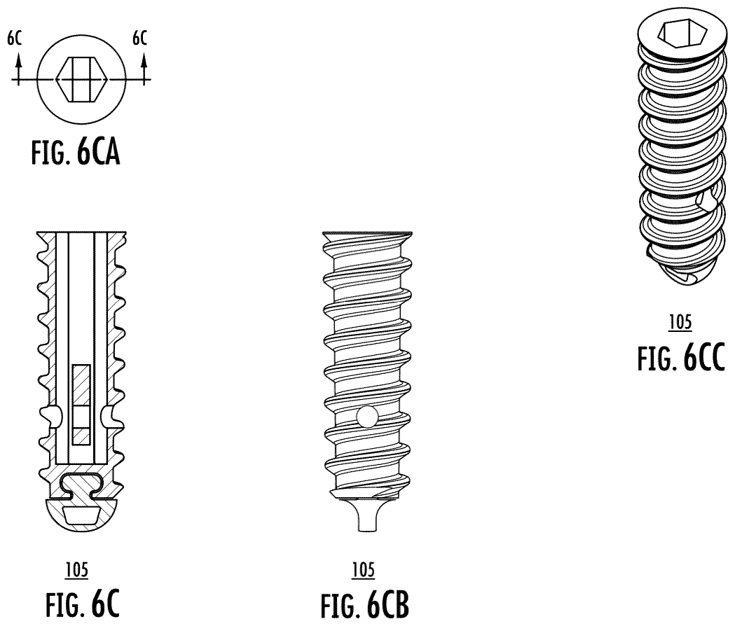

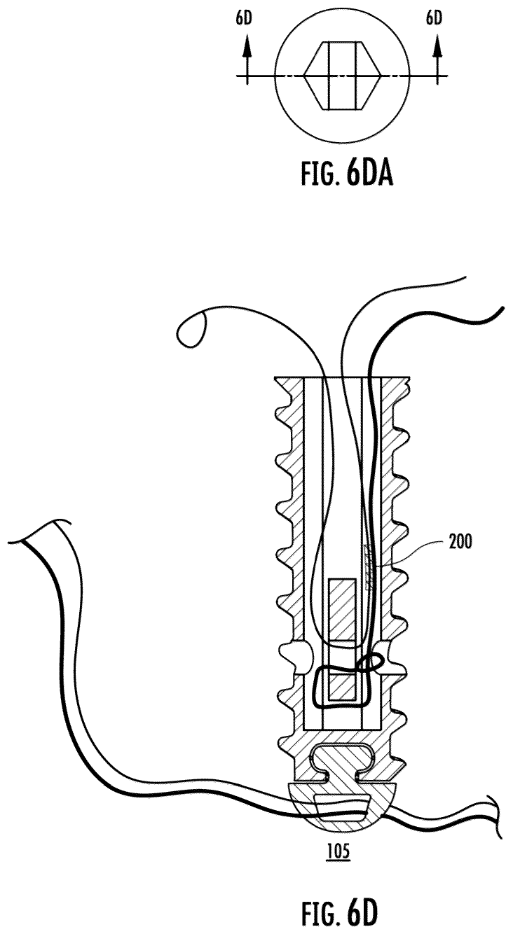

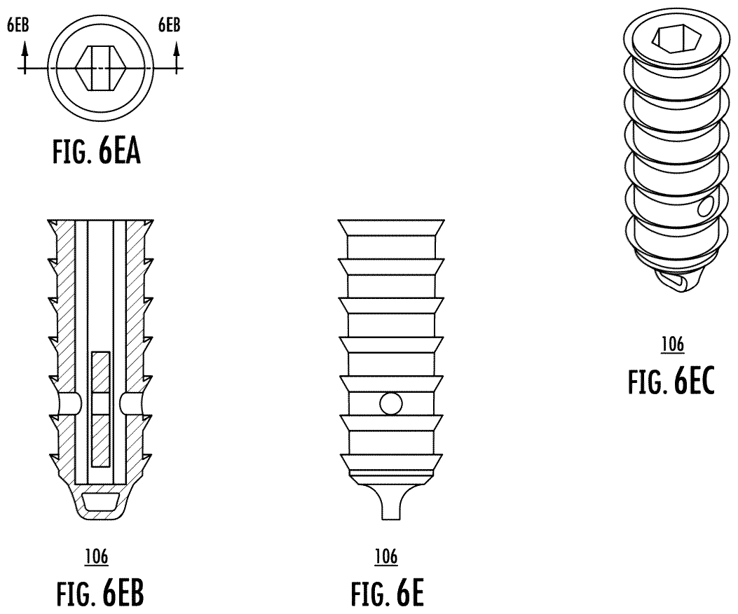

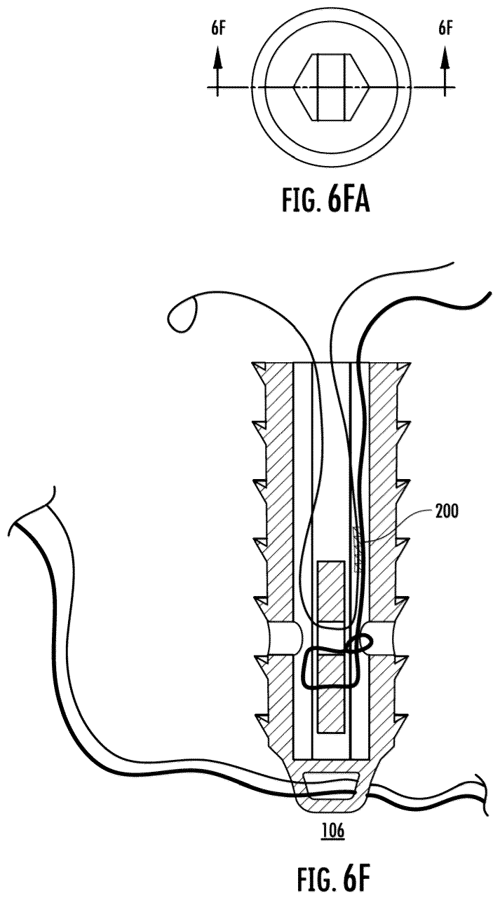

FIG. 6A illustrates a fifth embodiment of a suture anchor, namely suture anchor 05. Suture anchor 105 may include the same, similar, or substantially the same features and components as explained previously. As illustrated, suture anchor 105 may include a pivotable first component, or body member, 60 having a passageway 61 in which a corresponding first working suture 1 and first shuttle suture 2 may be passed therethrough. The second component 65 may be the same as, similar too, or substantially the same as any of the relevant anchoring sutures 100, 101, 102, 103, etc. disclosed herein. For example, the second component 65 may include a unidirectional sleeve 202 or the like, although it is to be understood that a variety of unidirectional suture locking configurations could be used instead of or in addition to sleeve 202, including without limitation, elongated suture entwinements, Chinese finger trap type devices (a type of woven sleeves), pre-tied knots, and hangman nooses. The example suture anchor 105 is configured to provide tension to a first working suture and a second working suture. For example, the first component 60 may be configured to provide and maintain tension to a first working suture 1 and the second component 65 may be configured to provide and maintain tension to a second working suture 1.

In use, a surgeon may first create a corresponding hole in bone by utilizing a drill. Thereafter, the surgeon may attach the first working suture 1 to adjacent soft tissue of a patient as explained previously. The first component 60 may be pushed down into the hole to a first initial depth to retain the first component 60 in place. Once the first component 60 is positioned in place (or sometime before) the surgeon may attach the second working suture 1 to a different location or different soft tissue of the patient. These steps may be performed out of order, e.g., the surgeon need not always position the first component 60 within the hole in boney anatomy first, e.g., they may be secured simultaneously.

The suture anchor 105 may be rotatably driven into the pre-existing hole of a corresponding diameter to secure and maintain the tension of the first working suture 1. In this embodiment, by rotating the suture anchor 105 and first portion 60, the first working suture is tensioned by a friction fit against the side surfaces of the boney anatomy. Thereafter, the surgeon may tension the second working suture 1 as explained previously with respect to suture anchor 100. For example, by pulling on the free end 2A of the shuttle suture 2 such that the unidirectional sleeve 202, or other unidirectional suture locking mechanism as discussed or contemplated herein, will maintain tension on the second working suture.

FIG. 6B illustrates a variation of the embodiment shown in FIG. 6A. In FIG. 6B, suture anchor 106 may include the same, similar, or substantially the same components and functionality as previously explained above with respect to suture anchor 105. In this embodiment, suture anchor 106 may include a plurality of fins 10 to enable this anchor to be tapped into boney anatomy rather than rotatably driven into boney anatomy.

FIGS. 6C-6DA are production drawings of the embodiment of FIG. 6A, where FIG. 6C is a sectional view of anchor 105 taken along the arrows shown in plan view FIG. 6CA, FIG. 6CB is an elevational view of suture anchor 105, FIG. 6CC is a perspective view of anchor 106, and FIG. 6D is a sectional view taken in the direction of the arrows in plan view FIG. 6DA. FIGS. 6E-6FA are production drawings of the embodiment of FIG. 6B, where FIG. 6EB is a sectional view of anchor 106 taken in the direction of the arrows shown in plan view FIG. 6EA, FIG. 6E is an elevational view of suture anchor 106, FIG. 6EC is a perspective view of anchor 106, and FIG. 6F is a sectional view of anchor 106 taken in the direction of the arrows in FIG. 6FA.

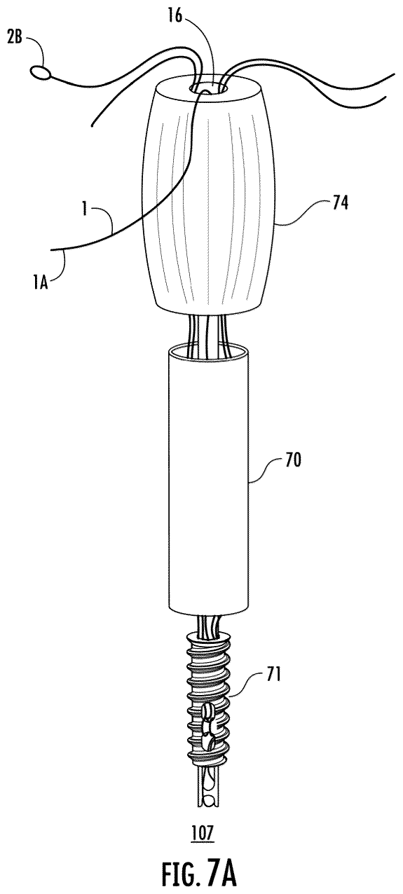

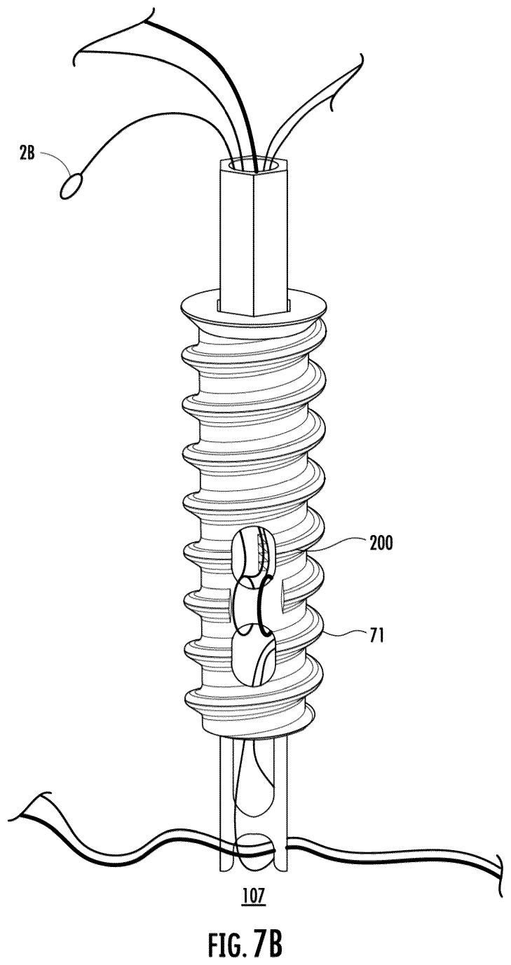

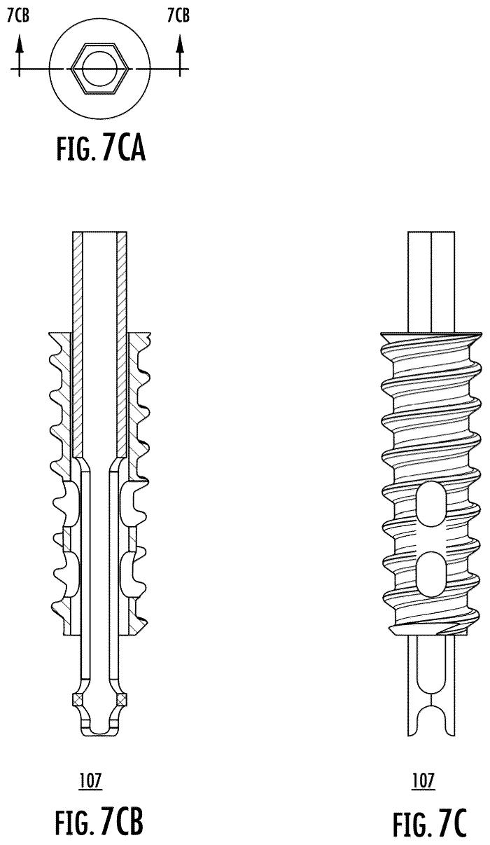

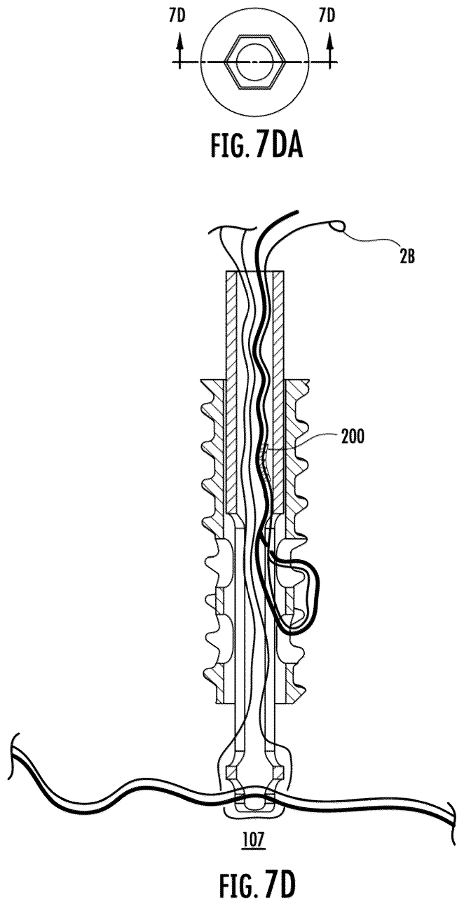

FIGS. 7A-7B illustrate a sixth embodiment of a suture anchor 107. Suture anchor 107 may include the same, similar, and or substantially the same features and functionality as explained previously. Suture anchor 107 may include a first component 71 that may be the same as or similar to any of the earlier explained suture anchor embodiments, e.g., 100, 101, 102, 103. First component 71 may include at least one working suture 1 and shuttle suture 2. Some embodiments may include a plurality of working sutures 1 and shuttle sutures 2. The working suture(s) 1 and shuttle sutures (2) may extend upwards through a corresponding passageway (not labeled), up through a sheath or sleeve that may protect the suture(s) during the installation thereof. The second component 74 may then slip down over the sheath and the first component 71. At this point, the surgeon may tighten any number of working suture(s) 1 via any number of corresponding shuttle suture(s) 2 as explained previously. FIG. 7B is an enlarged view of first portion 71 with example commentary and explanation of parts. Suture anchor 107 also includes one or more unidirectional suture locking mechanisms (not shown) as discussed or contemplated herein. FIGS. 7C-7DA are production drawings of the embodiment of FIGS. 7A-7B, where FIG. 7CB is a sectional view taken in the direction of the arrows in plan view FIG. 7CA of anchor 107, FIG. 7C is an elevational view of anchor 107, and FIG. 7D is a sectional view taken in the direction of the arrows in FIG. 7DA.

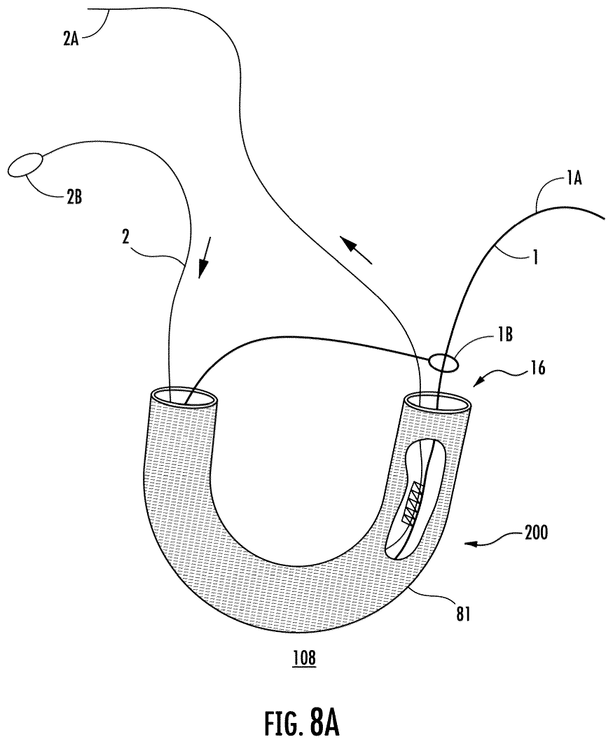

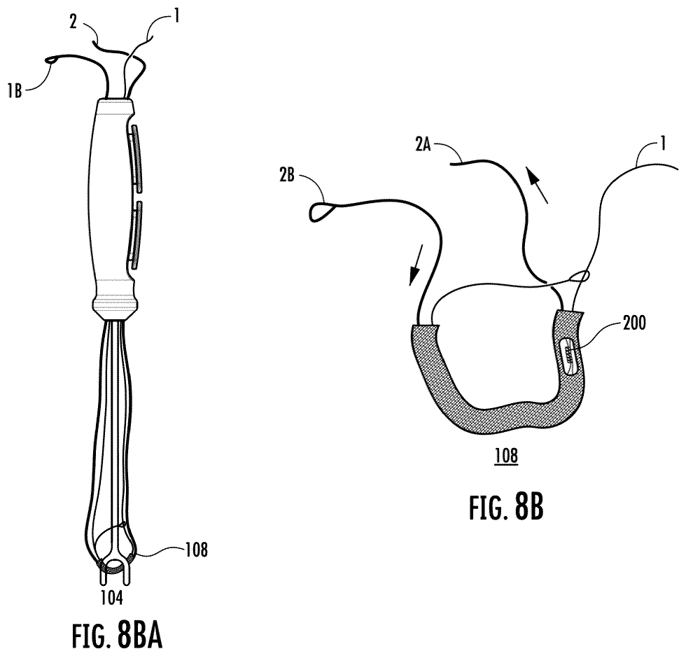

FIGS. 8A-8BA illustrate a seventh embodiment of an insertable flexible tubular suture shell 108, which could be constructed of suture or other material and which itself forms a unidirectional suture locking mechanism, i.e., a knotless mechanism for maintain tension of a suture. Suture shell 108 is, in contrast to the generally rigid anchors, or “shells,” 100, 101, 102, 105, 107, and 107 discussed above, is pliable and manipulatable in form. In this embodiment a tool, which may take the general shape of a fork, and in particular, a pickle fork 80, may be used to push a suture shell 81 down into a pre-existing hole in boney anatomy of a patient. The suture shell 81 may bunch or ball up, i.e., wrinkle or fold, or “scrunch up”, etc., to expand outwardly and bind itself via an interference fit and form a U-shape in the pre-existing hole, while still maintaining an internal passage sufficient to carry multiple sutures. The outside surface of the suture shell 81 may be a high friction surface that is useful for retaining the suture shell 81 within the pre-existing hole by a friction fit. The internal passage is configured to only allow passage of one or more sutures in a first predetermined direction, and attempts to pull one or more of the sutures in a second direction opposing the first direction causes fibers, yarns and/or other elements within the internal passage to engage, grip and restrain the one or more sutures from movement in the second direction. Additionally, the working suture 1 and shuttle suture 2 may be passed through a central passageway 16 of the shuttle suture 81 as illustrated.

With the suture shell 81 in place, a surgeon may tighten the working suture 1 similarly as explained above. For example, a surgeon may loop the open end 1A of the working suture through the looped end 2B of the shuttle suture and/or attach the working suture 1 open end 1A to the shuttle suture 2 looped end 2B. Thereafter, the surgeon may pull the shuttle suture open end 2A upwards and away from a passageway 16 extending through the suture shell 81. In doing so the looped end 2B of the shuttle suture 2 and the open end 1A of the working suture are pulled downward into the passageway 16 (at least marginally). Given the free end 2A of shuttle suture 2 has been preloaded through the suture shell 81 (see FIG. 4D), continued pulling of shuttle suture 2 upwardly (in the orientation of suture anchor 103 shown in FIG. 4D) eventually draws both shuttle suture looped end 2B and working suture 1 downwardly through the bottom of the U-shape of shell 81 and then upwardly through the remainder of shell 81, and still further upward pulling of shuttle suture 2 causes shuttle suture 2 to exit shell 81, with working suture 1 passing by itself through shell 81. In doing so, the shell 81 acts as a unidirectional suture locking mechanism to bind and thereby maintain the tension in the working suture 1 in a manner similar in effect as discussed above regarding other unidirectional suture locking mechanisms. In doing so, the suture shell 108 may maintain a tension in the working suture 1 similarly as explained above.

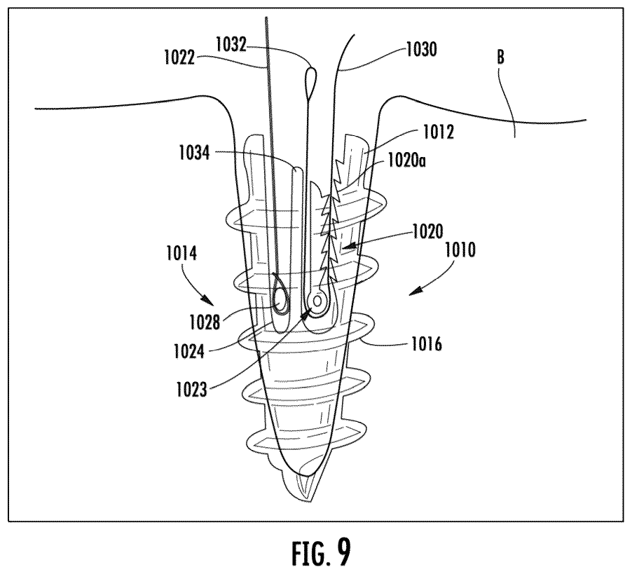

Referring now to FIGS. 9-12, additional embodiments of a knotless suture anchor (also referred to herein as “suture anchor” and as simply “anchor”) with an internal suture locking mechanism of the present disclosure is indicated generally in FIGS. 9-12by reference character 1010.

As shown in FIGS. 9-11, suture anchor 1010 includes a shell, generally 1012, which may be similar to conventional suture anchors. Shell 1012 may include, in one preferred embodiment, a screw-in portion 1014 having threaded portions 1016 and/or a tap-in configuration (not shown) with either barbs or threads, based on whether anchor 10 is to be tapped in or screwed into the bone, generally B.

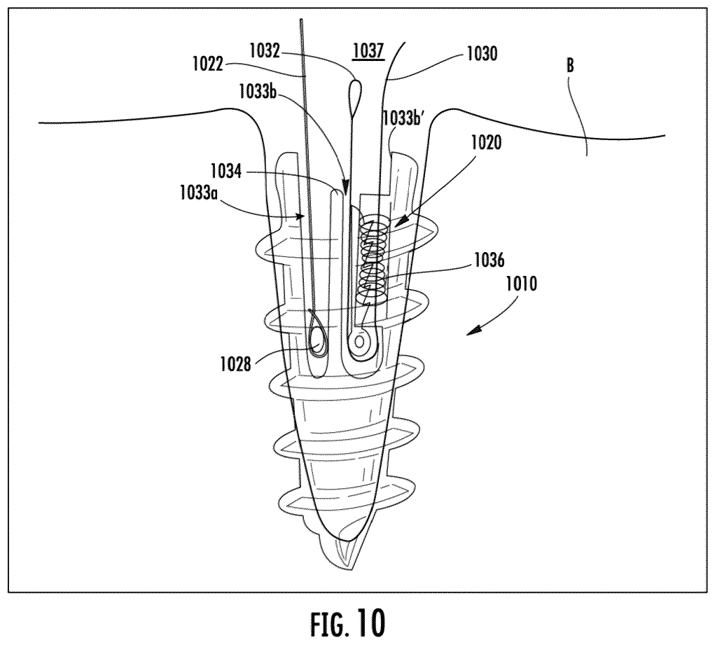

Body member, or shell, 1012 may be constructed of a non-absorbable plastic polymer, bioabsorbable plastic polymer, metal, or some other suitable and implantable material. An internal mechanism, generally 1020, of anchor 1010 may include one or more high-strength braided nylon sutures, generally 1022, with one end of such suture 1022 being fixed, as shown in FIGS. 9 and 10, in an elongated passage 1033a, which extends generally parallel to an elongated passage 1033b, both passages 1033a, 1033b being defined by a guide 1034 in the body, generally 1024, of anchor 10. Such fixation of suture 1022 to body 1024 can be done in various ways. One way includes actually fusing suture 1022 to a polymer portion of body 1024. Other ways may include using some sort of post, such as post 2108, about which suture 1022 can be securely looped around and tied within anchor 1010.

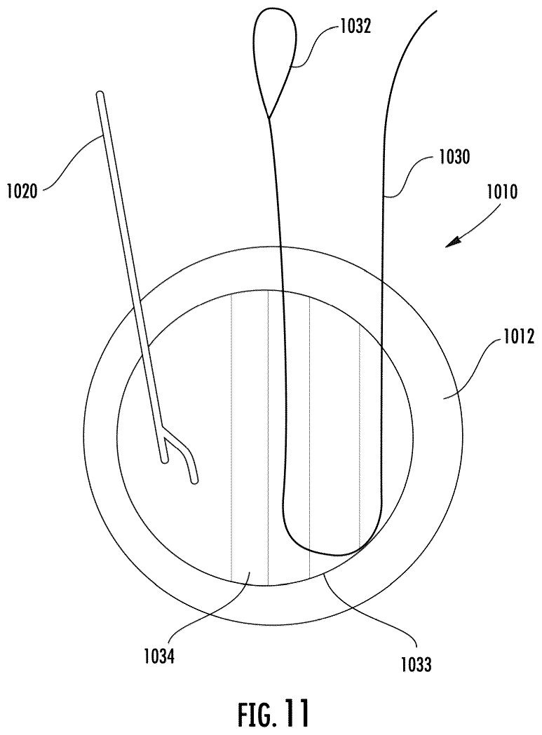

As shown in FIG. 11, in addition to suture 1022, anchor 1010 may also include, in one preferred embodiment, a heavy monofilament nylon shuttle suture, generally 1030, placed in anchor 10. Shuttle suture 1030 has two free ends, one having a suture loop 32 which provides a shuttle through anchor 1010 once the loop 1032 has been passed through tissue (not shown). Once loop 1032 is passed through anchor 1010 via a passage 1033b defined by guide 1034, internal locking mechanism 1020 then allows both the suture shuttle and the braided nylon suture 1022 (which passes through loop 1032 of shuttle suture 1030) to be pulled through anchor 1010 in a uni-directional fashion, with internal locking mechanism 1020 cinching suture 1022 by acting as an internal locking cleat 1023, wherein suture 1022 can pass one way but not the other. Accordingly, as the free end of shuttle suture 1030 is gradually pulled, it initially passes through internal locking mechanism 1020, but eventually, with continued pulling of shuttle suture 1030, suture 1022 passes into passage 1033b and through internal locking mechanism 1020 to ultimately cinch the tissue down (the tension may be adjusted in the tissue tension “rolling” tissue in either direction), such that the tissue is pulled down to the bone B in and/or near the suture anchor 1010. This allows re-approximation of the attachment of the tissue to the bone and allows adjustment of the tension both within suture 1022, and thus also the tissue to attach itself to bone B.

Internal locking mechanism 1020 can be of various configurations, and the present disclosure is not to be limited to the mechanisms disclosed and/or shown herein. Multiple other internal locking mechanisms could be used in anchor 1010, and the internal locking mechanisms disclosed and/or shown herein are for illustrative purposes and are not intended to limit the scope of the internal mechanisms that could be used in connection with anchor 1010.

One variation of internal locking mechanism 1020 could be a series of opposing resilient, directional barbs, or teeth, 1020a which allow suture 1022 to be pulled in between teeth 1020a in only one, i.e., a “first” direction. An attempt to pull suture 1022 in the other (“second”) direction would cause the teeth 1020 a to engage and hold suture 1022 against further movement in the second direction. However, in the event suture 1022 is again pulled in the first direction, teeth 1020a would release suture 1022 to allow suture 1022 to again be pulled in the first direction. Internal locking mechanism 1020 thus allows passage of sutures 1030, 1022 in one direction, but not in the opposite direction.

Another variation of internal locking mechanism 1020 could be an internal pre-tied locking knot, such as a hangman's noose knot 1036 adjacent a projection 1037 along a sidewall 1033b′ of passage 1033b, as shown in FIG. 10. As also shown in FIG. 10, shuttle suture 30 passes downwardly in passage 1033b in a first direction and then reverses direction before passing upwardly into and through knot 1036.

Still another variation could be a woven suture inside a suture anchor which once again allows uni-directional passage of the braided nylon suture. This could be a configuration similar to a Chinese finger trap type of device, which tightens against the suture moving in a predetermined direction.

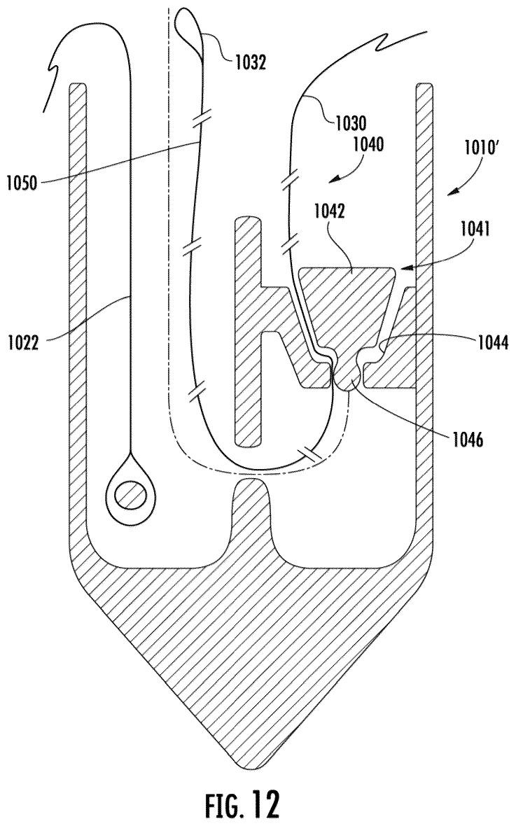

Other variations could include, but are not limited to, a conical locking mechanism, generally 1040, as shown in anchor 1010′ in FIG. 12. Conical snap locking mechanism, generally 1041, includes a generally conical member 1042 which is biased by weight and/or spring force towards a seat 1044. Although not shown, a strap, cable, line, or the like could be used to prevent member 1042 from falling out of anchor 1010′. Suture 1022 may pass in a first direction whereby member 1042 is caused to be lifted slightly above seat 1044, to thereby form a passage for suture 1022. If suture 1022 is pulled in the other (“second”) direction, however, member 1042 automatically moves downwardly, with neck portion 1046 ultimately seating in a snap-fit, interference-fit arrangement with respect to seat 1044 and engages suture 1022 and fixes it against movement in the second direction. Similarly, as discussed above with respect to other versions of internal locking mechanism 1020, in use, the surgeon would pass suture 1022 through the patient's tissue and then load suture 1022 in and through loop 1032. The free end of shuttle suture 30 would then be pulled until suture 1022 (moving in the first direction) passes through anchor 10. Tension would be applied in suture 1022 until adequate tension is achieved, and then the other free end of a second, line or suture, 1050, the “locking limb,” which has its other end connected to member 1042, is pulled in the second direction until the conical snap locking mechanism 1041 locks suture 1022 in place. Excess suture extending outwardly from the tissue is then cut and removed.

Further, a cam mechanism (not shown) could be provided that flips back to lock upon itself in the event suture is pulled in reverse fashion.

It is to be understood that the above various internal locking mechanism configurations could be used either singularly or in combination with one another in a given anchor 10, if desired.

Conventional knotless anchor designs generally marry the angle of insertion of the anchor to a portal, since the anchor has to be placed through the portal, and then suture passage through tissue must be achieved. At this point, the anchor position must be re-found through the same portal. Then, the tissue is cinched down. However, for certain positions, in the shoulder in particular, this creates a technical problem, because frequently it is in an inappropriate location to place a portal. For example, in repairing a superior labrum or SLAP lesion, anchors are frequently placed percutaneously through the rotator cuff. It is advantageous in this situation to make these perforations through the rotator cuff as small as possible. However, normally a 6-8 mm diameter cannula is required to be inserted through this tissue in order to position conventional cinching knotless suture anchor designs.

With anchor 1010 of the present disclosure, anchor 1010 could be placed percutaneously through a 3 or 4 mm hole, and then the surgeon would have the freedom to pass the suture through tissue in a variety of other angles within the shoulder. This would likely minimize damage to the adjacent tissue, particularly with respect to the rotator cuff noted in the example above. Labral repair is but one application of the present disclosure. Anchor 1010 could also be used in other repairs, such as rotator cuff repair in the shoulder, biceps tenodesis, and a myriad of other procedures in the shoulder and elsewhere. Anchor 1010 could be used in any other joint in the body in which suture anchor placement is required for soft tissue fixation to bone.

In short, anchor 1010 provides a versatile device which could be used in the shoulder, in particular, and for numerous other applications in the musculoskeletal system to facilitate soft tissue fixation to bone, with minimal suture damage. The present disclosure may find particular applications in procedures involving the: shoulder (labrum, rotator cuff, biceps tenodesis, etc.); knee (backup hamstring fixation, MCL/LCL reconstruction, ligament avulsions, etc.); elbow (UCL/LUCL reconstruction, biceps, etc.); foot and ankle; and wrist. The present disclosure also provides a lower profile by eliminating prominent knots on articulating surfaces.

Aspects of the present disclosure may include a braided nylon suture with one end fixed to an anchor, a heavy monofilament nylon suture shuttle, internal locking mechanisms with multiple variations, and a simplified methodology of how a knotless suture anchor with an internal suture locking mechanism may be constructed in accordance with the present disclosure might be inserted and used. Also disclosed is a simplified technique for insertion and use of an anchor of the present disclosure. For example, in methods disclosed herein, suture 1022 can be single or double loaded as applicable.

Anchors 1010, 1010′ can be produced in various diameters, including without limitation, diameters between approximately 2 and 6 millimeters.

The present disclosure thus provides, among other things, the potential of decreased surgical time, increased surgical accuracy, a decreased dependence on knot-tying and loosening, lower profile by eliminating prominent knots on articulating surfaces.

Specifically, the knotless anchor of the present disclosure may be inserted through drilling cannula (which reduces the likelihood of losing the hole), and a “normal” suture passage may be used. Also, tissue tension may be adjusted by “rolling” tissue in either direction. This can thus address problems which may occur with current anchor designs, such as losing the hole in the methodology where a hole is drilled, and the devise is removed. Then, the hole must be relocated. This can pose particular difficulty in relation to the lower glenoid quadrants. Additionally, the angle of insertion of such a conventional anchor may be married to a working portal. Moreover, with such designs, it may be difficult to manipulate tissue as a suture is tensioned.

In one exemplary implementation, anchor 1010 may include a standard tap-in or screw-in insertion that permits drilling and insertion through the same cannula, which may be 3.0 to 4.5 mm cannula, and as noted above, the anchor may be single or double loaded. Anchor 1010 may include suture 1022 being a wire fixed on one end in the anchor and with a free end. A heavy nylon suture shuttle may be preloaded, and an internal locking mechanism may include a cleat, a locking knot, and a Chinese finger trap type device. Other variations of the present disclosure could include a Chinese finger trap type device including a suture-in suture with unidirectional tensioning, an internal cam, and a second suture to lock a pre-tied knot when adequate tension has been obtained.

As explained above, exemplary implementations disclosed herein describe cinchable suture anchors which are useful for attaching soft tissue to bone. Multiple versions of anchors are described herein, including “hard” anchors (typically made of solid materials loaded with sutures) and “soft” anchors (typically made of various types of suture material or fabric and loaded with sutures).

The anchors described herein are useful in that they can be applied to attach soft tissue to bone without the use of knots. Also, the cinching mechanisms of the anchors described herein allow the user to progressively increase tension through the tissue, thus bringing the tissue closer to a target point and allowing the surgeon to customize and finely adjust the compression force to each particular application.

The anchors described herein can be made in various sizes and of various materials. Examples of the hard anchor shell materials would include, but are not limited to, PEEK and absorbable biocomposite polymers. Examples of the soft anchor shell would include, but are not limited to, ultra-high molecular weight polyethylene, nylon, polyester and other suitable polymers for suture or fabric, and including absorbable materials.

To recap the disclosure above, the basic technique by which the anchors herein are utilized involve the following steps: insertion of the anchor into bone; passing the working suture attached to the anchor through the soft tissue which is to be attached; using a shuttle suture with a looped end and a free end to pass the tail of the working suture through the cinching mechanism of the anchor; pulling on the working suture tail until adequate tension is achieved which, in turn, applies a compression force to the soft tissue onto the bone.

The purpose of this compression is to facilitate healing of the soft tissue to the bone. The anchors disclosed herein can be used both in open surgical procedures and in arthroscopic surgical procedures. The insertion tools disclosed herein are constructed so that these anchors would be useful in both open surgery and arthroscopic procedures.

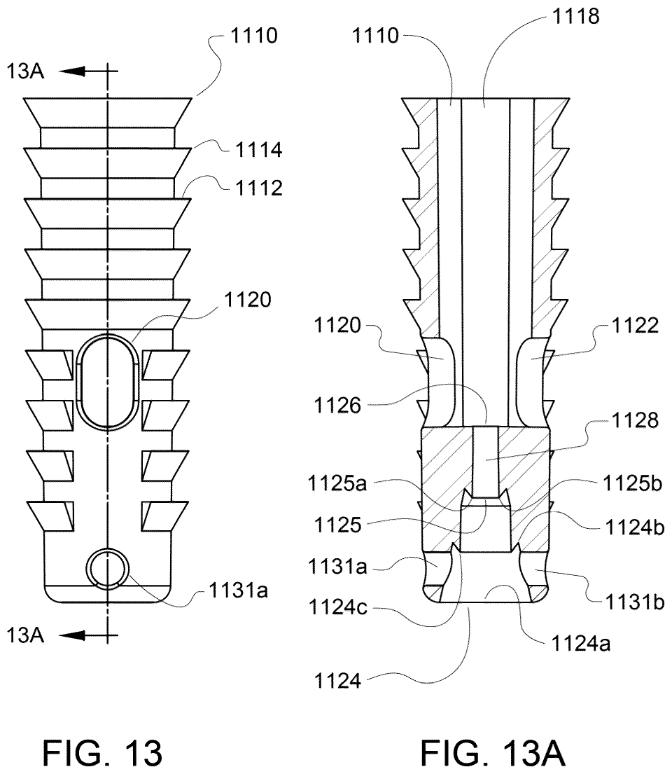

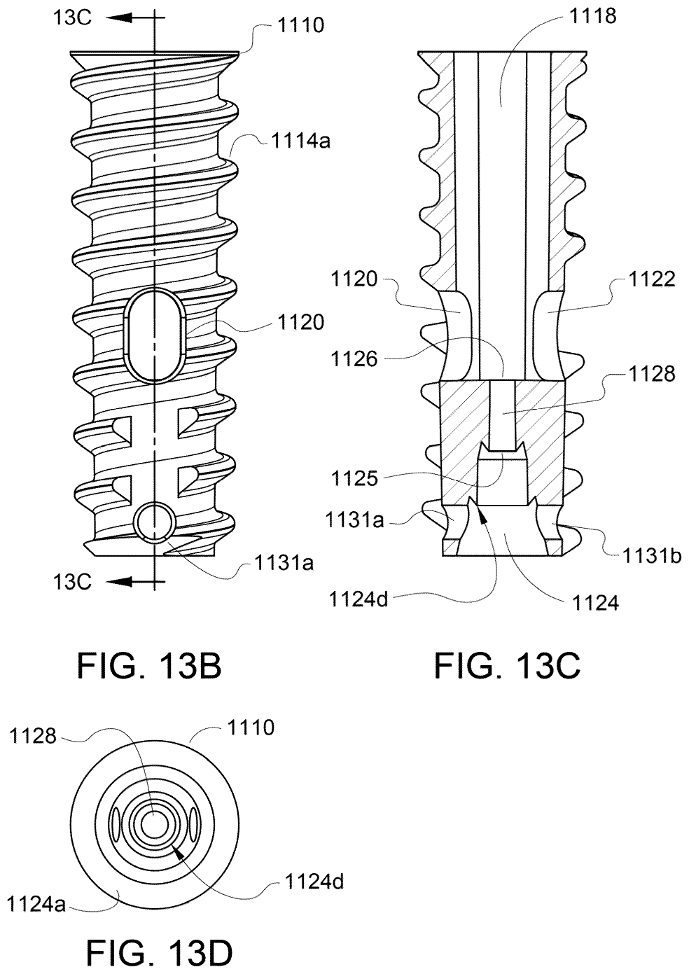

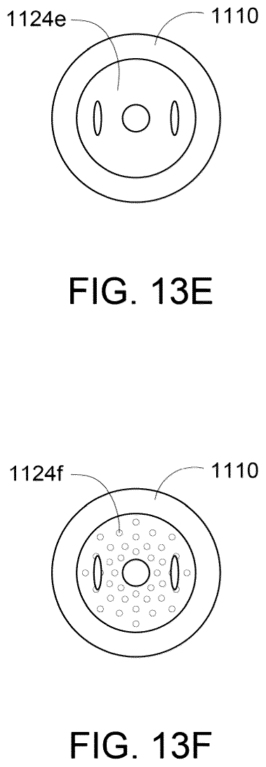

Turning to FIGS. 13-13F, another implementation of a suture anchor, namely, anchor 1110, is provided for implantation in tissue configured for receipt of a suture, with the anchor includes a body portion having a bottom portion and defining a passage, or opening, 1124 in the bottom portion configured for receipt of the suture. The body portion of anchor 1110 defines a passage 1125 in communication with the opening 1124 which is configured for receiving the suture. At least one of the opening 1124 and the passage 1125 is progressively narrowed and configured to form a cinching arrangement to induce friction in the suture as the suture is pulled through the cinching arrangement, whereby the suture may be selectively fixed with respect to the anchor 1110 as the suture is pulled through the cinching arrangement.

More specifically, anchor 1110 includes a hard outer shell 1112 equipped with outer fins 1114, or threads 1114a (FIGS. 13B and 13C) suitable for inserting into bone. Fins 1114 of outer shell 1112 are configured such that when anchor 1110 is tapped into a predrilled hole, fins 1114 attach anchor 1110 to the bone. Instead of fins, the anchor 1110 may be equipped with threads 1114a to allow anchor 1110 to be screwed into a predrilled hole. The proximal shape of an upper portion 1116 (which is in communication with a central passage, generally 1118) of anchor 1110 is shown as being generally hexagonal in shape which fits a hexagonal driver to tap or screw the anchor into bone.

Anchor 1110 is designed with suture openings, or windows, 1120 and 1122 to allow a suture to pass through the anchor 1110. A passage 1124 is provided in the bottom of anchor 1110 and is specially shaped to increase friction as the suture passes through the cinching arrangement or mechanism, generally 1126. The bottom of anchor 1110 can be of various shapes, which include, but are not limited to conically-shaped, funnel-shaped, bell-shaped, dome shaped, box shaped, pyramidal or triangular shaped, etc. in communication with a suture passage 1128 for receiving and allowing suture to pass therethrough, upon the suture first passing through passage 1124 and then through a passage 1125, which is narrower than passage 1124. Note that passage 1128 is narrower than passage 1125, which aids in enhancing the frictional engagement of the suture with the walls of passage 1128 and the friction between adjacent suture, thereby “cinching” the suture as the suture is pulled through passages 1124, 1125, and 1128, to restrain rearward or retrograde movement of the suture from such passages.

As shown in FIG. 13D, a bottom view of one implementation of anchor 1110 may include a configuration where the shape and surface of the anchor bottom interacts with the working suture and in the cinching mechanism of the anchor. Various shapes produce various levels of friction within the cinching mechanism. FIG. 13D illustrates a cone-shape version of passage 1124 having raised teeth 1124d. The shape and depth of the cone can be varied to alter the performance characteristics. The suture, including the working suture and pre-tied knot or luggage tag, interact with this surface and teeth 1124d, which increases friction of the suture in the cinching mechanism.

More specifically, as shown in FIG. 13D, surface 1124a of the bottom can be varied as well and can be smooth or have ribs, teeth, projections, protuberances and/or textured, etc. The surface features 1124a help to increase friction being applied to suture passing through cinching mechanism 1126 while still allowing for smooth movement of suture through suture passage. Note that a circular recess or groove 1124b is provided at the juncture of passage 1124 and 1124a, which provides a downwardly extending ringed tooth 1124c for introducing more friction in the suture as it is pulled and makes its way from passage 1124 into passage 1124a. Similarly, a circular recess or groove 1125a is provided at the juncture of passage 1124a and 1125, which provides a downwardly extending ringed tooth 1125b for introducing more friction in the suture as it is pulled through passage 1124a into passage 1125.

FIG. 13E illustrates a bottom view of an implementation of anchor 1110, where the cone inlet portion 1124e of anchor 1110 is smooth. Once again, passage 1124 can be varied in depth and shape in numerous ways, meaning, it does not have to be a perfect cone but instead can have convex or concave profile, a hemispherical profile, a bell-shaped profile, an inverse funnel profile, or a combination of such profiles. Altering the shape of the bottom can produce various levels of friction within the cinching mechanism as desired.

FIG. 13F illustrates a bottom view of an implementation of anchor 1110, where the cone inlet portion 1124e of anchor 1110 has been textured with numerous projections or protuberances and/or dimple type shapes 1124f. This influences the level of friction as the suture is tightly applied against this surface.

Central passage, or cannula, 1118 includes in the implementation narrow passage 1125, which allows a suture to pass from the top of anchor 1110 to the bottom of anchor 1110 but is narrowed so as to not allow a knot or multiple sutures to pass upwardly from the bottom to the top of anchor 1110 or in a reverse, or retrograde, direction.

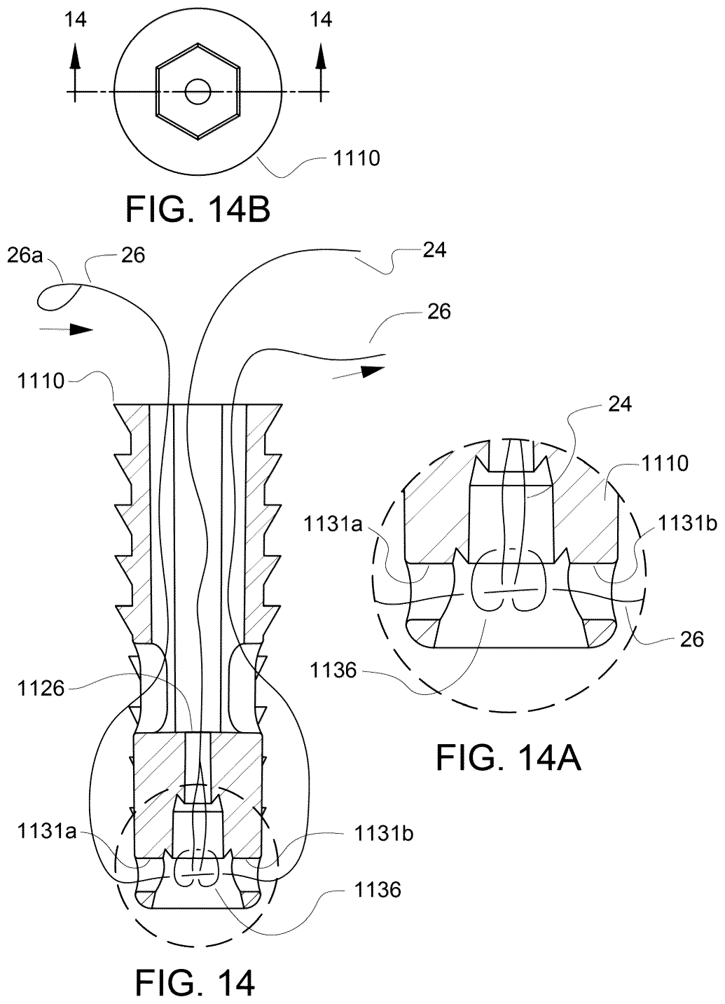

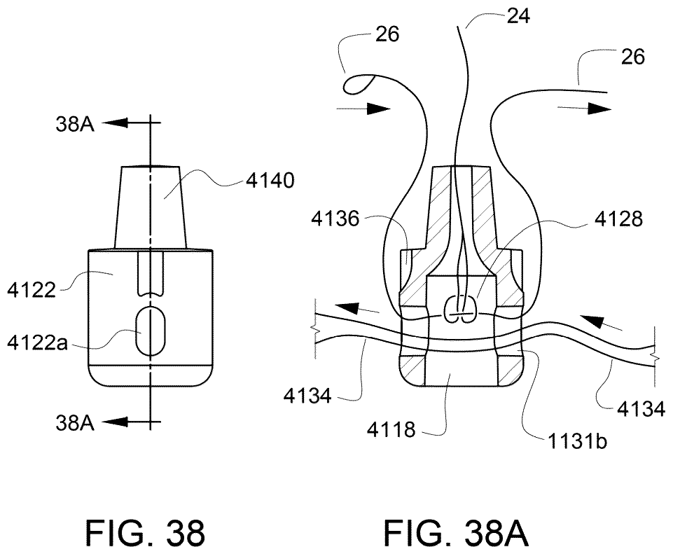

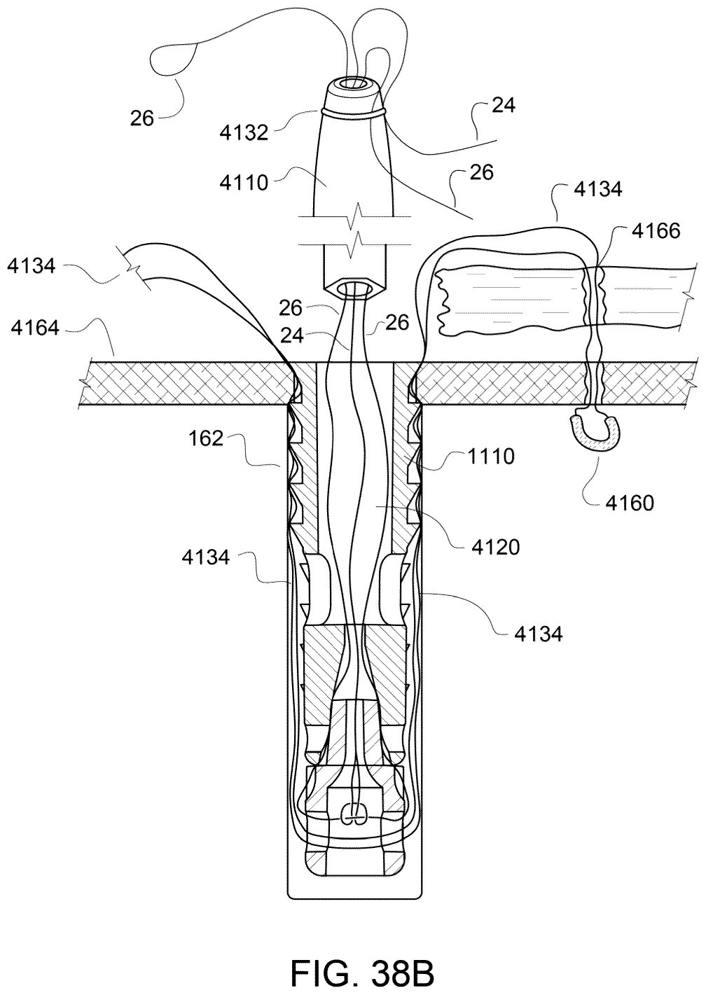

Fenestrations, slots, or windows, 1131a and 1131b are provided in the lower portion of anchor 1110 and are in communication with generally cone-shaped passage 1124 and to vary the suture paths of working sutures 24 and shuttle sutures 26 as needed for a particular application or implementation, such as discussed herein with respect to FIGS. 14-19 , and similarly, in modified fashion, in FIGS. 38A and 38B.

In FIGS. 14, 14A, 14B, 15, 15A, 15B, 16, 16A, and 16B, exemplary implementations of anchor 1110 is shown equipped with suture. A working suture 24 and a shuttle suture 26 are shown. The shuttle suture 26 is equipped with a small loop 26a at the bottom end of suture 26. Loop 26a can be formed into pre tied knots of various shapes. Working suture 24, can be of various diameters and of various cross sections, including flat oval and round, and working suture 24 may be tapered to allow easier passage through the cinching mechanism 1126.

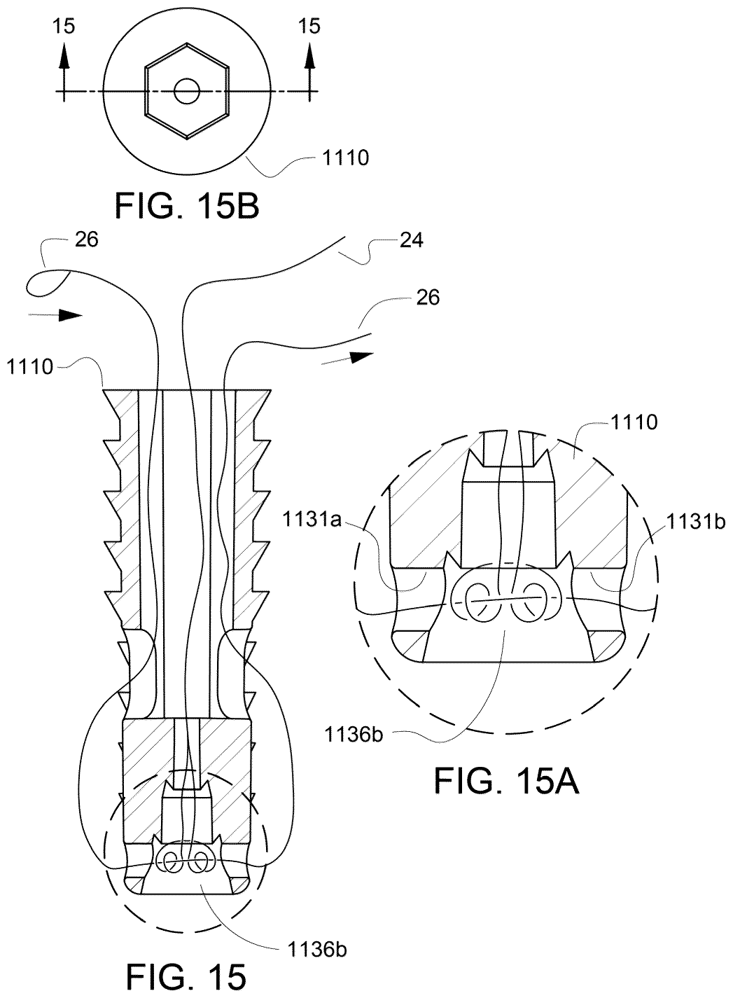

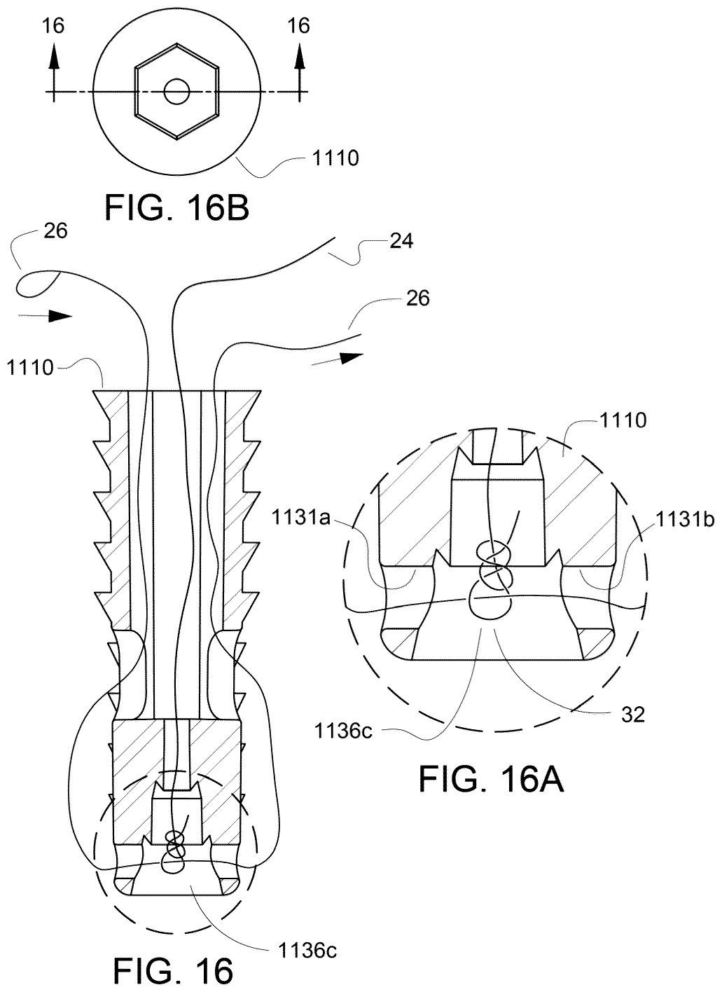

As shown in FIG. 14-14B, an exemplary implementation of cinching mechanism 1126 may include, but is not limited to, a pretied knot, including a luggage tag pre-tied knot, generally 1136. This pretied knot, or luggage tag, 1136 of working suture 24 is wrapped around the shuttle suture 26. When there is no tension on the working suture 24, there is very little friction on the shuttle suture 26. When the shuttle suture 26 is used to pass the working suture 24 back through the anchor 1110 through a bottom chamber 1124 of anchor 1110 and the luggage tag 1136, there is increasing friction on working suture 24 as it is pulled through the anchor 1110. As described previously, in the usual application, the working suture 24 has been passed through soft tissue, returns back through the anchor via the shuttle loop 26a through the cinching mechanism 1126 located in the bottom of the anchor 1110 and through the luggage tag, or other pre-tied knot configuration. This arrangement forms a continuous suture loop which progressively tightens, i.e., the more tension that is placed on the working suture 24, the more friction it generates within the anchor 1110, and cinching mechanism 1126, which only permits uni-directional pulling of working suture 24, meaning cinching mechanism 1126 will tension working suture 24, but it will not allow working suture 24 to loosen under normal conditions. Examples of the pre-tied knot in the bottom portion of anchor 1110 are shown in FIGS. 14 and 14A as a single luggage tag 1136, as a double luggage tag 1136b in FIGS. 15 and 15B, and as a noose-type pre-tied knot 1136c in FIGS. 16 and 16A.

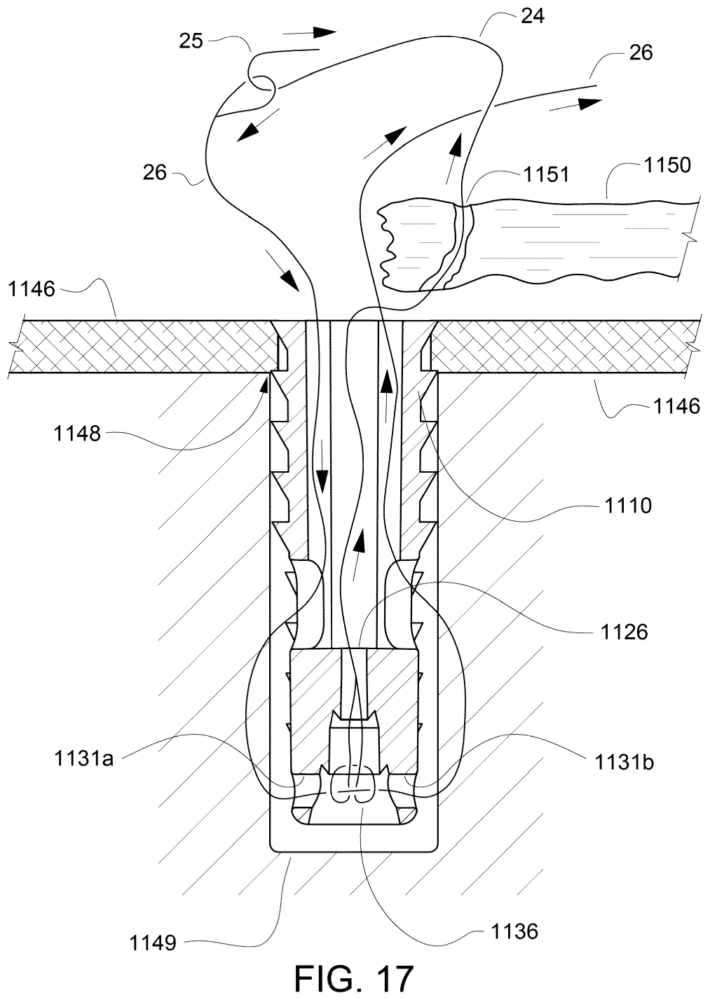

In FIG. 17, an exemplary implementation of anchor 1110 has been placed in a pre-drilled hole 1148 to a depth of 1149. The outer table or a cortical layer of bone 1146 is usually harder than the underlying bone which allows for better fixation. The working suture 26 has been passed through the tissue 1150 at point 1151, and the tail of suture 26 has been passed through the loop 26a of suture shuttle 26 and folded over, as shown at point 25. This prepares the working suture 26 to be shuttled through the anchor and its locking, or cinching, mechanism, generally 1126. The directional arrows in FIG. 17 show how the suture 26 will be pulled through the anchor cinching mechanism 1126.

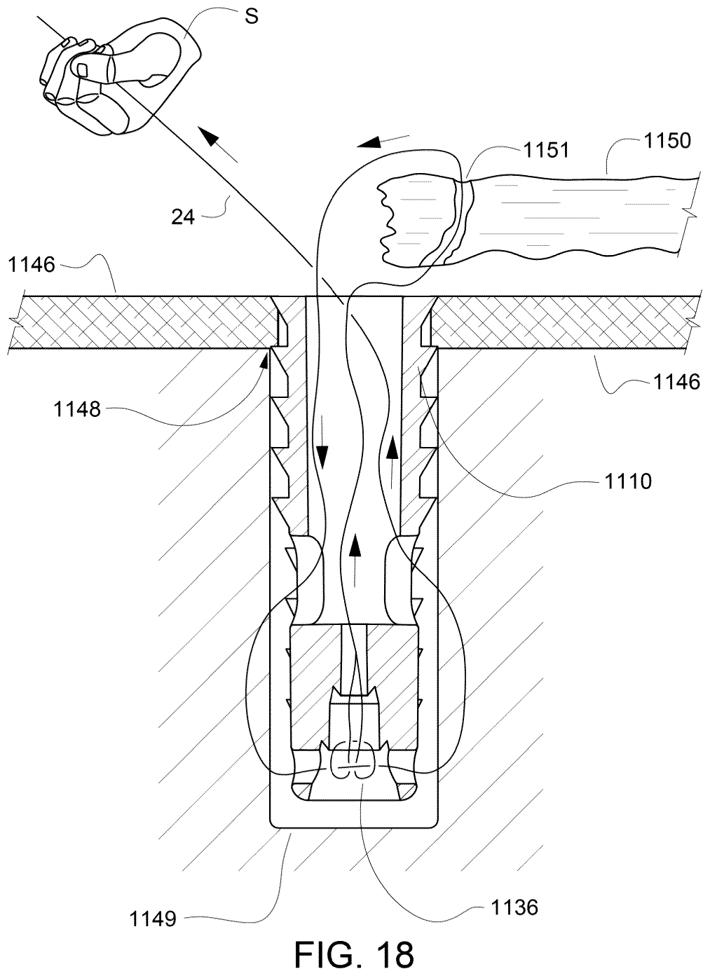

In FIG. 18, an exemplary implementation of anchor 1110 of FIG. 17 is shown after shuttle suture 24 has been shuttled through the anchor 1110. The surgeon simply pulls on suture 24 in a manner similar to using a zip tie, until adequate tension is achieved. This places a compressive force on tissue 1150 to the bone 1146 and a tension force on the anchor 1110.

FIGS. 19, 19A, and 19B show an exemplary implementation of anchor 1110 of FIG. 14, with an additional knot 1134 placed in the working suture 24. This knot 1134 creates additional friction and improves resistance to loosening once the working suture 24 is deployed through the anchor 1110.

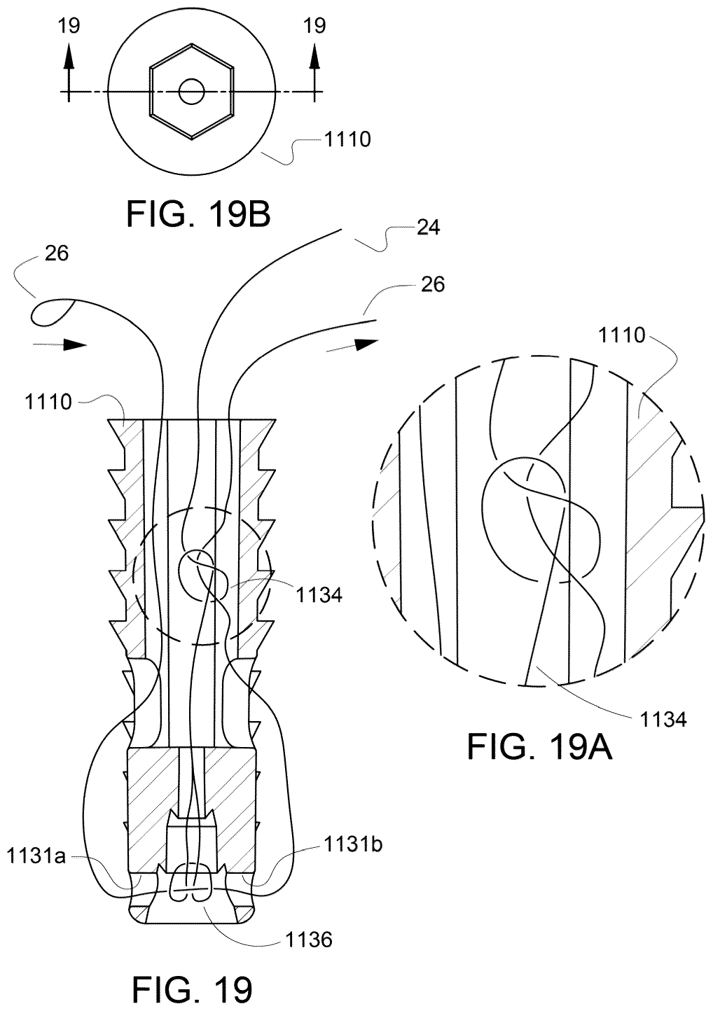

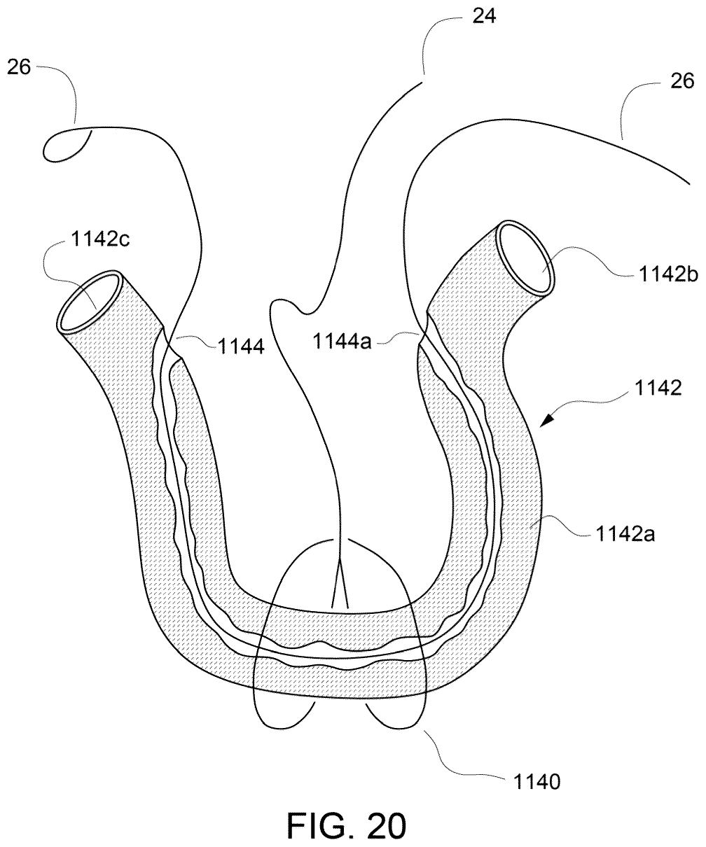

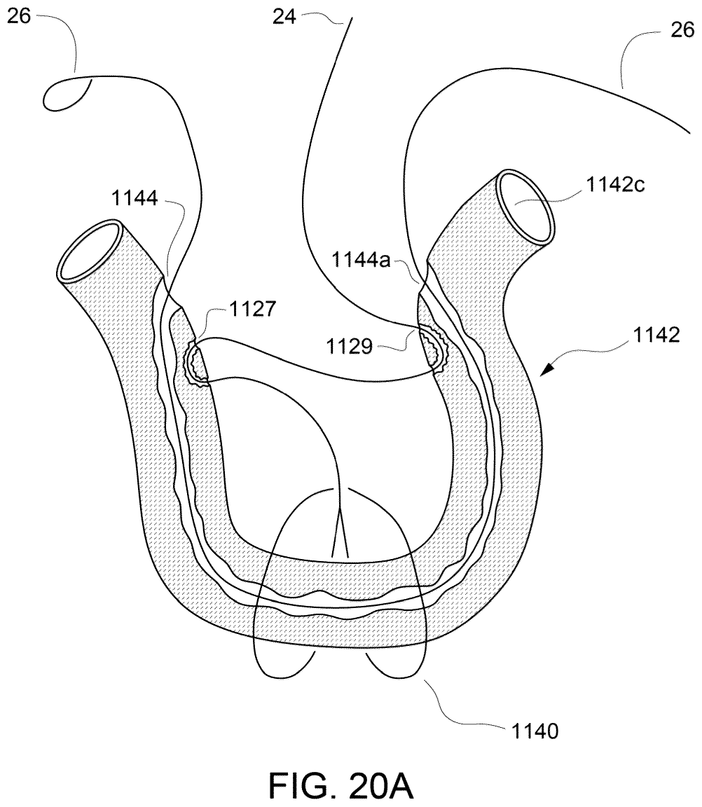

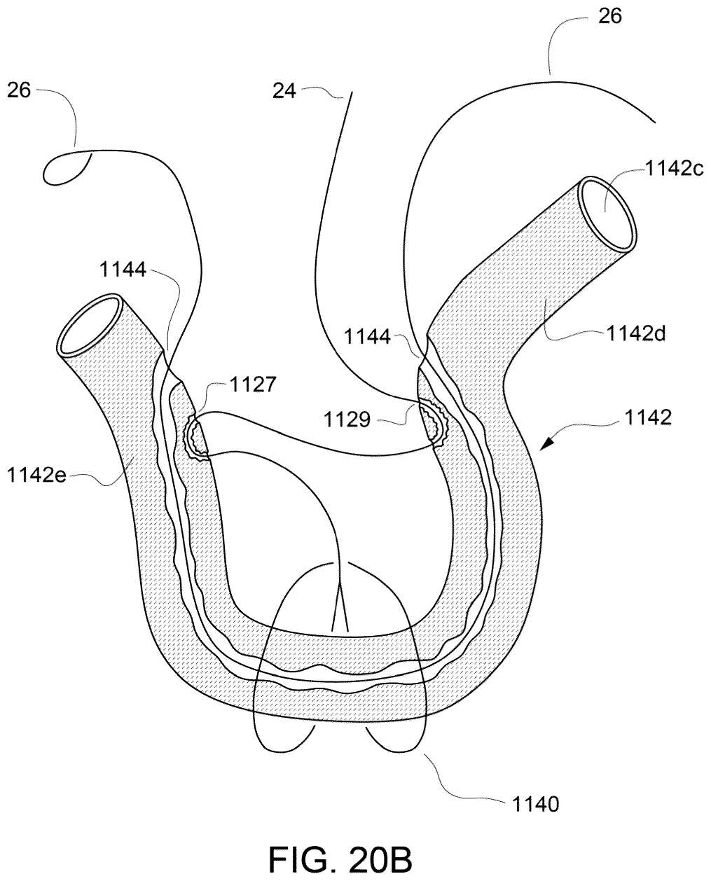

In FIGS. 20 and 20A, an exemplary implementation of a soft anchor 1142 is shown configured for receipt of a suture and for implantation in a boney tissue hole, where the anchor 1142 includes a pliable body portion 1142a constructed of at least one of ultra-high molecular weight polyethylene, nylon, polyester and other suitable polymer material, and the body portion is configured for receipt of the suture and for insertion into the boney tissue hole. The body portion is configured for expanding outwardly, in a sense, by crumpling “bunching up” and/or “balling up,” upon tension being applied to the suture, where, upon the body portion being inserted in the boney tissue hole and tension being applied to the suture, the body portion bunches up or balls up to expand sufficiently to effectively fix the body portion 1142a in the hole by an interference fit, and consequently, the suture to the anchor. In certain implementations, the body portion is constructed of a woven material, nonwoven material and/or an extruded or seamed material.

More specifically, anchor 1142 is shown in FIGS. 20 and 20A as including the body portion or shell 1142a, which may be constructed of suture material or woven fabric constructed of a suitable flexible material, including, but are not limited to, ultra-high molecular weight polyethylene, nylon, polyester and other suitable polymers for suture or fabric. Shell 1142 a could also be of a smooth or textured tube or tube-like extruded or seamed construction not of woven or nonwoven fabric. This would also include by absorbable materials. Shell 1142a allows fixation to bone when anchor 1142 is pushed into a pilot hole, such that anchor 1142 becomes fixed when tension on sutures 24 and 26 causes the shell 1142a to effectively compress and expand outwardly to bunch up, or, “ball up,” so that anchor 1142 cannot pull back out of the bone. This permits a high level of fixation to the bone. Anchor 1142 is equipped with a working suture 24 that is analogous to the working suture in the hard anchors discussed above and otherwise herein. Working suture 24 wraps around suture itself by means of a pre-tied knot, namely, a luggage tag type knot 1140. The shuttle suture 26 passes through the wall 1142b of the shell 1142a at points 1144 and 1144a. Points 1144, 1144a can be varied in their distance from the ends the anchor 1142 as desired for different applications. Additionally, as shown in FIG. 20A, working suture 24 can pass through the wall 1142b of the shell 1142a at points 1127 and 1129, which can be varied in their distance from the ends the anchor 1142 as desired for different applications.

FIG. 20B illustrates an implementation of soft anchor 1142, which is identical to anchor 1142 shown in FIG. 20A, except that the lengths of the proximal and distal ends of the anchor 1142 are asymmetrical. One limb or arm 1142d has been made longer than the other limb or arm 1142e. This may be advantageous in certain applications and help to improve fixation of anchor 1142 in bone.

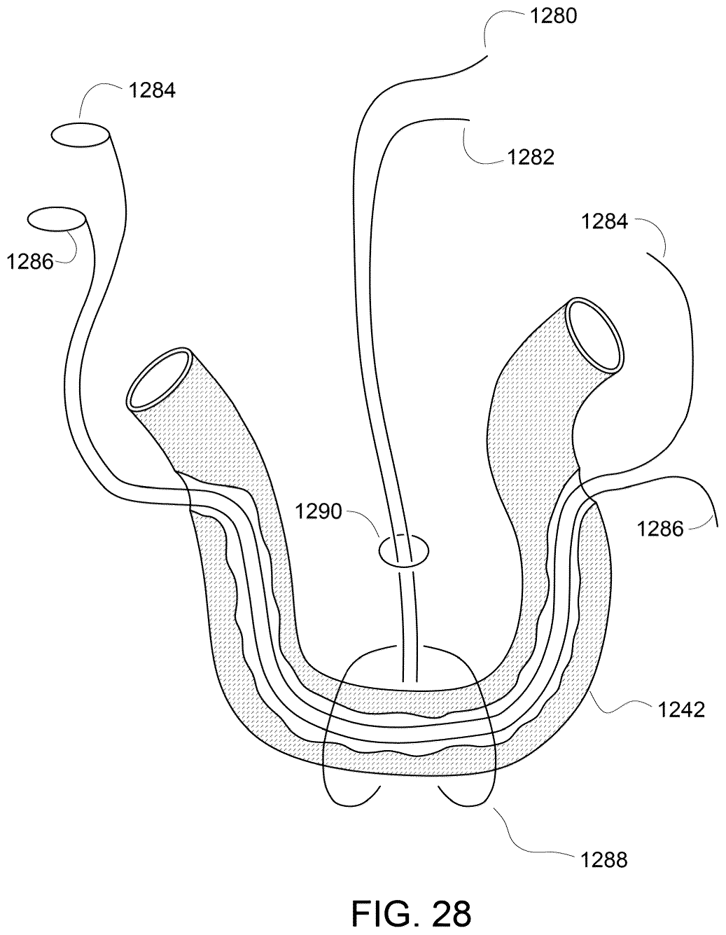

FIG. 28A illustrates an implementation of double-armed soft anchor 1143, the working suture 84 is attached to the body of the anchor 1143 via a luggage tag pre-tied knot 1289. Working suture 80 passes through the shell 1143a of one arm of the anchor 1143 at location 1127. The second working suture 82 is attached to the anchor via luggage tag 1288. It then passes through the anchor shell 1143a at location 1129. The anchor 1143 contains two shuttle sutures 84, 86. Once the anchor 1143 is deployed in bone, the working sutures 80 and 82 are shuttled through the anchor independently with their respective shuttle suture, 84 or 86, respectively. A variation of this use of this double-armed anchor is to use only one shuttle suture, either suture 84 or suture 86, to shuttle both working sutures 80 and 82 through the anchor 1143 simultaneously. This simplifies the procedure and still allows the working sutures 80 and 82, to be tensioned independently of one another. Another advantage of this configuration is that when the anchor 1143 is implanted in bone, tensioning of the working sutures 80 and 82 helps to bunch-up the anchor and give it a high level of fixation in a predrilled hole in which anchor 1143 is deployed. Notably, anchor 1143 can be useful in double row tendon repairs.

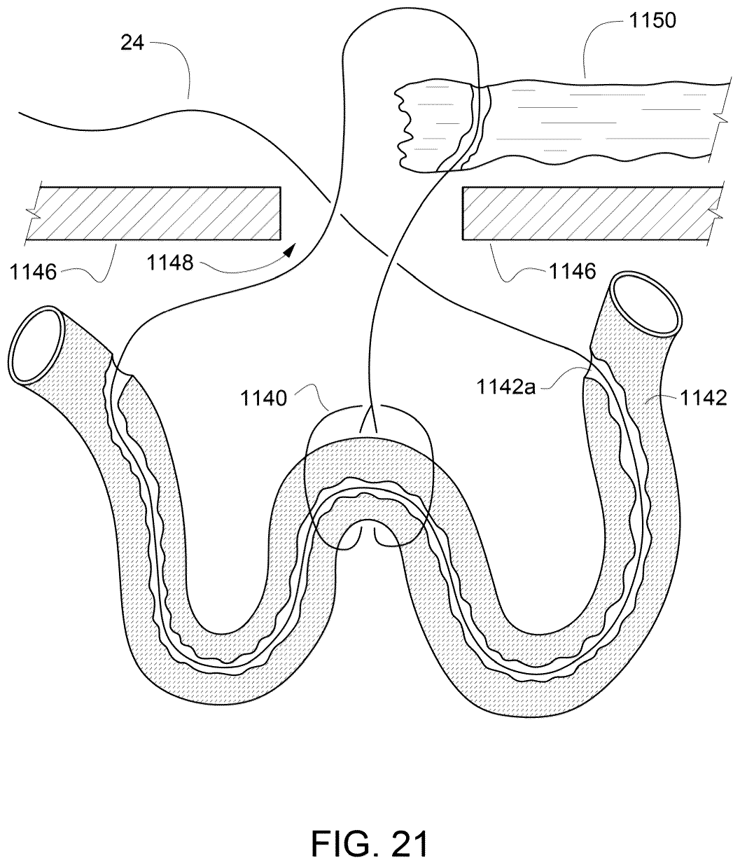

In FIG. 21, the position of the anchor 1142 is shown after it has been deployed in bone 1146. Living bone typically has a hard outer cortical shell and deeper layers of cancellous, or softer bone. In this application, a hole 1148 is drilled through the cortical shell into the lower softer bone. The suture anchor 1142 is passed through hole 1148, and when tension is applied to the working suture 24 and the shuttle suture 26, anchor 1142 changes shape and, in at least one exemplary implementation, effectively balls, or, bunches, up an/or turns into a wave, wavy, or generally “W” shape. Once anchor 1142 is fixed and the working suture 24 has been shuttled through anchor 1142, the shape of the construct, or configuration, of anchor 1142 and sutures 24 and 26 are as shown in FIG. 21-25. Note that the working suture 24 passes from the luggage tag pre-tied knot 1140 through the hole 1148, through the tissue 1150, back through the passage 1142c (which extends the length of anchor 1142) through the luggage tag 1140 (which encircles sheath 1142a though which suture 24 passes while in central passage 1142c), and then out of and out hole 1144. During the movement of working suture 24 in this manner, the surgeon S continually applies tension to the tail of the working suture 24 until adequate pressure on the tissue 1150 is achieved, and this, in turn, causes luggage tag knot 1140 to pinch or cinch working suture 24 withing anchor 1142 to thereby restrain further movement of working suture 1124. This arrangement is referred to herein as the fixation, or cinching, mechanism of soft anchor 1142. Once adequate tension and the attachment of tissue 1150 to bone 1146 have been achieved, the tail of working suture 24 is cut, and the tissue repair is complete. The fixation mechanism of working suture 1142 is provided via the friction forces encountered by working suture 24 due to its torturous path through anchor 1142 and the tension on the luggage tag knot 1140 compressing working suture 24.

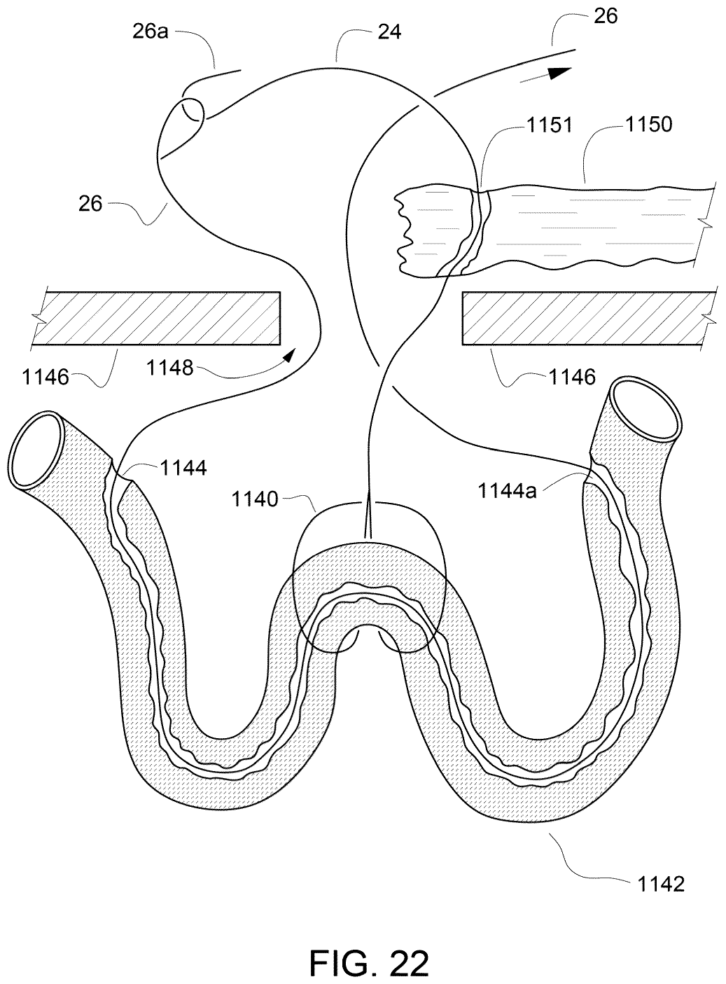

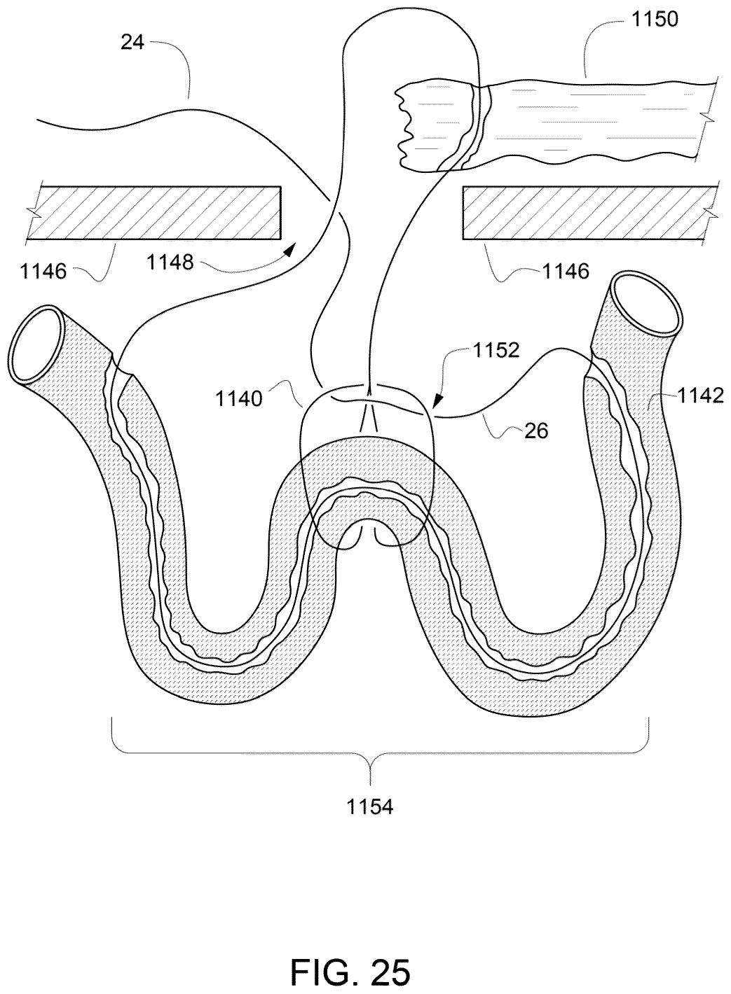

FIG. 22 shows another exemplary implementation of anchor 1142 of FIG. 21. Anchor 1142 has been passed into bone 1146 and deployed so that the anchor 1142 has balled/bunched up into a “W” shape (FIG. 25) and is thereby substantially restrained from pulling out of bone 1146, and working suture 24 has been passed through tissue 1150 at location 1151. The tail of the working suture 24 has been passed through the loop 26a of the shuttle suture 26 as shown at point 26b. This prepares the arrangement for shuttling the suture 24 through anchor 1142 and the cinching of working suture to anchor 1142.

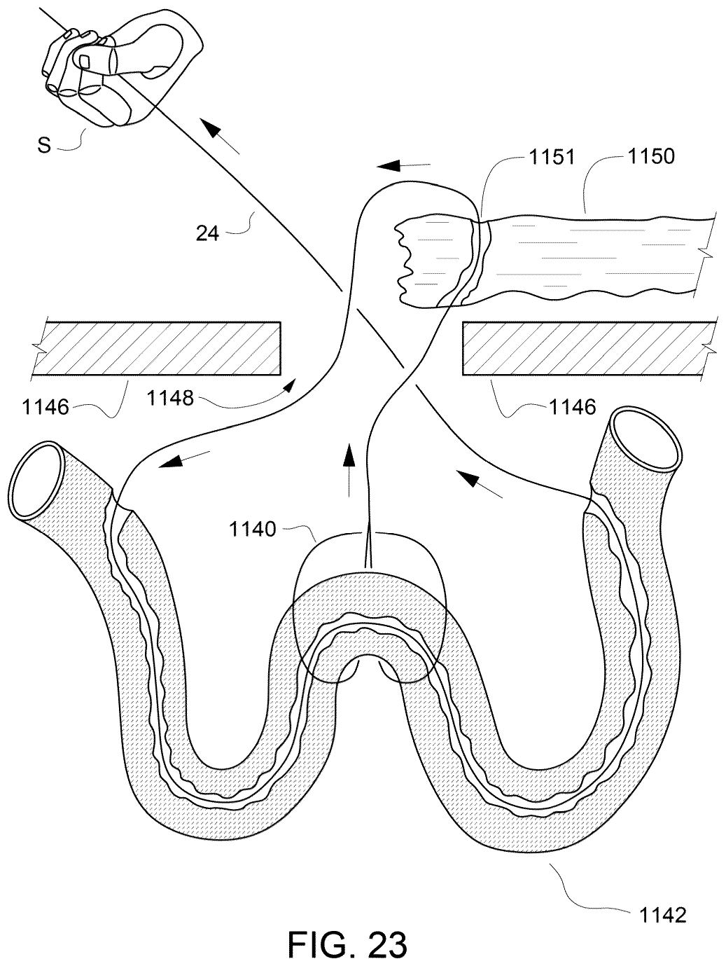

In FIG. 23, working suture 24 has been shuttled through anchor 1142 and the surgeon by hand 24d grips and pulls on suture 24 until appropriate tension is achieved. This creates compression of the tissue 1151 against the bone 1146 to promote healing of the soft tissue to the bone.

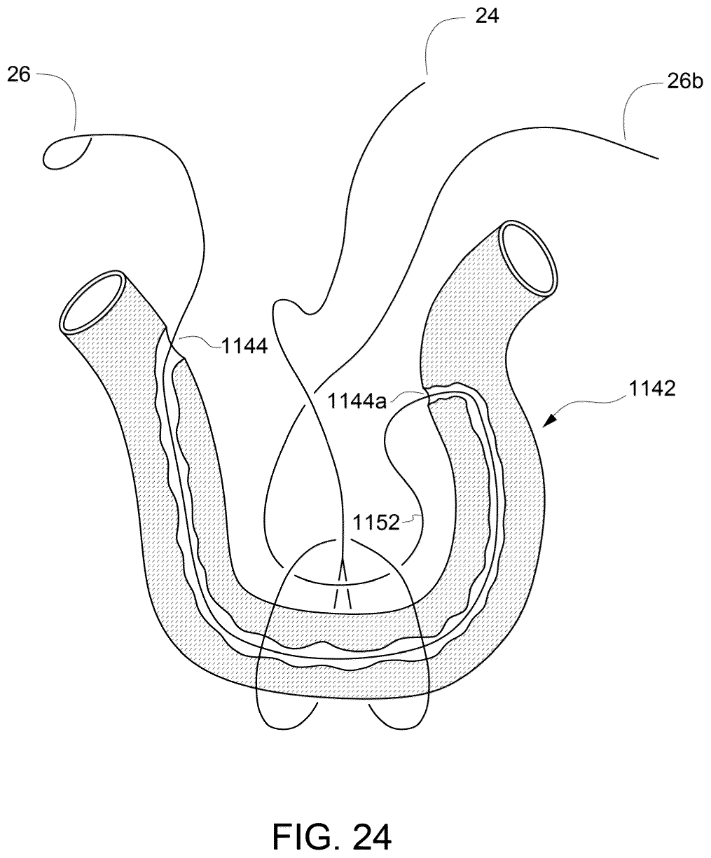

FIG. 24 represents another exemplary implementation of an anchor 1142. In this implementation, the free end 26b shuttle suture 26 passes through shell 1142 through the luggage tag knot 1140 and then exits shell 1142a. Once suture free end 26 exits shell 1142, a length the suture limb 1152 is turned back through the luggage tag knot 1140, and then the shuttle suture proceeds out of shell 1142a of anchor 1142. This extra turn back through the luggage tag knot 1140 allows for greater friction and greater balling/bunching up of the anchor 1142 in the hole in the bone. Other than the course of the exiting limb 1152 of the shuttle suture 26, this implementation of anchor 1142 is generally similar to implementations of anchor 1142 discussed above.

FIG. 25 schematically illustrates an exemplary implementation where the anchor 1142 shown in FIG. 24 after it has been deployed in bone 1146, where the working suture 24 has been shuttled back through the anchor 1142 with an additional pass through the luggage tag knot 1140 and then exits anchor 1142. The implementation of FIG. 25 is similar to the implementation of FIG. 21, which shows the anchor 1142 in its deployed state or configuration.

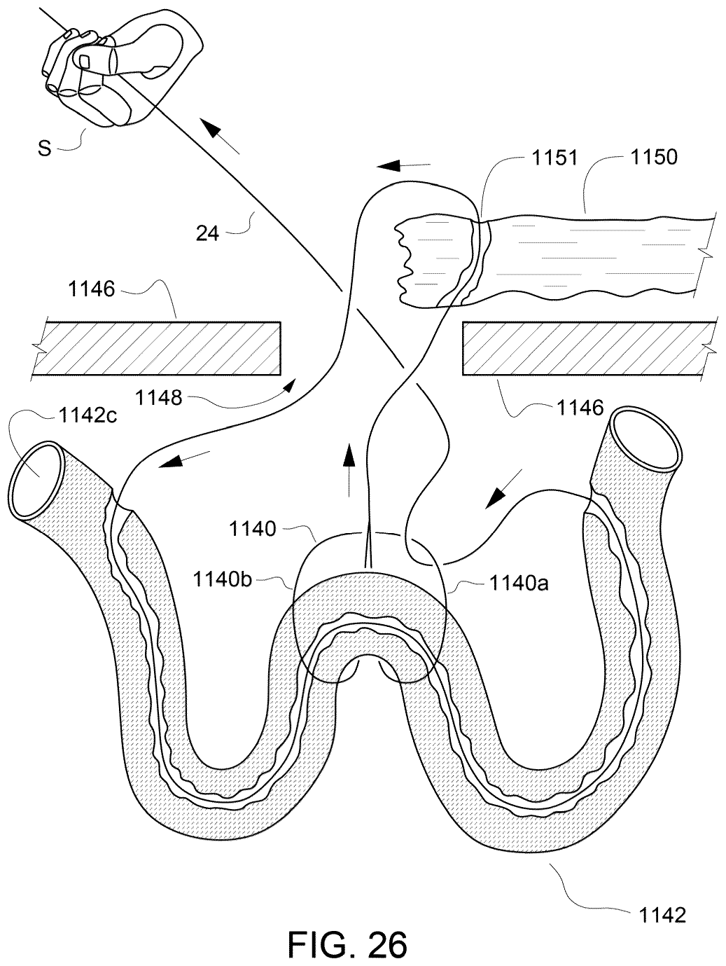

FIG. 26 shows another exemplary implementation of anchor 1142 shown in FIG. 25. Anchor 1142 is identical to the anchor deployed in FIG. 25, except the working suture 24 goes under only one limb 1140a of the luggage tag 1140, instead of under both limbs 1140a and 1140b as shown in FIG. 25.

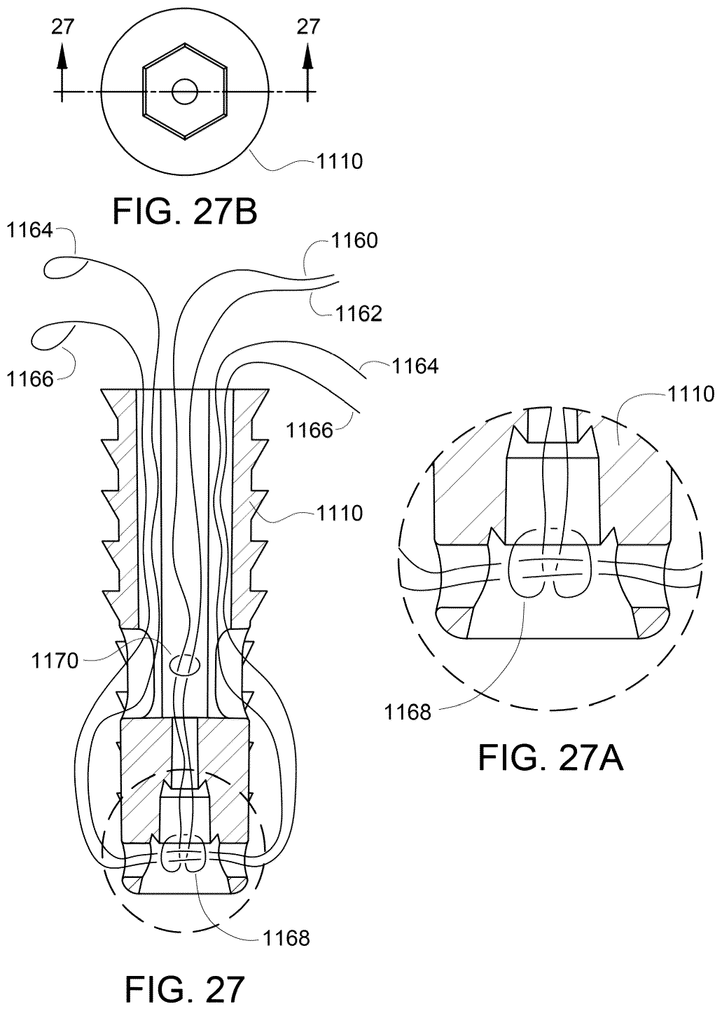

There are instances when it is desirable to have more than one working suture in a given suture anchor. In the implementations shown in FIGS. 27, 27A, and 27B, there are two working sutures 1160 and 1162 in anchor 1110, and two shuttle sutures 1164 and 1166. This arrangement is similar to the singly loaded anchor demonstrated in FIG. 14 and allows the first working suture 1160 to be passed through tissue and then shuttled back through the anchor via shuttle 1164 and then tensioned. In many cases, multiple tissue fixation points are needed for adequate repairs, thus, the second working suture 1162 would then be passed through tissue and shuttled back through the anchor via shuttle 1166 to give double fixation. This double fixation is arguably somewhat synergistic, meaning that increasing tension on working suture 1160 and working 1162 increases the friction and fixation of the anchor 1110. An additional feature is a knot 1168 which may be provided to restrain slippage of one working suture on the other, i.e., knot 1168 may be tied to limit slippage of suture 1160 on suture 1162.

FIG. 28 illustrates yet another exemplary implementation of a doubly armed soft cinching suture anchor 1242. This is a soft version of a suture anchor and has similarities with the anchor shown in FIG. 27. Anchor 1242 includes a working suture 80 and a working suture 82, with a shuttle suture 84 and a shuttle suture 86. The fixation method includes use of a luggage tag number pretied knot 88. This configuration allows, as with the anchor arrangement shown in FIG. 27 regarding use of anchor 1110, the creation of two fixation points through soft tissue 1150 using a single anchor. Anchor 1242 may be equipped with a knot 90, as well, which may restrain relative slippage between working sutures 80 and 82.

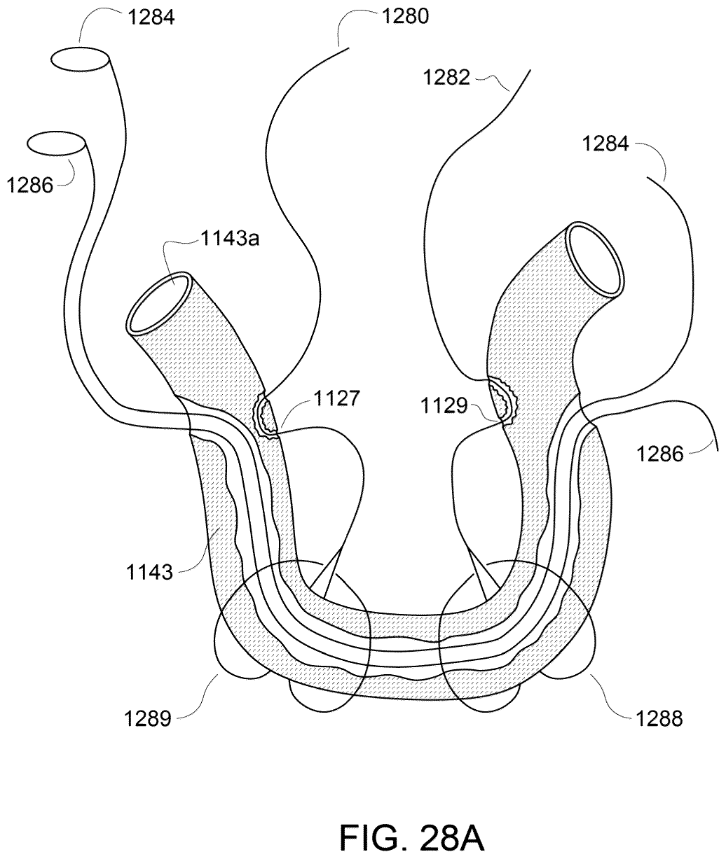

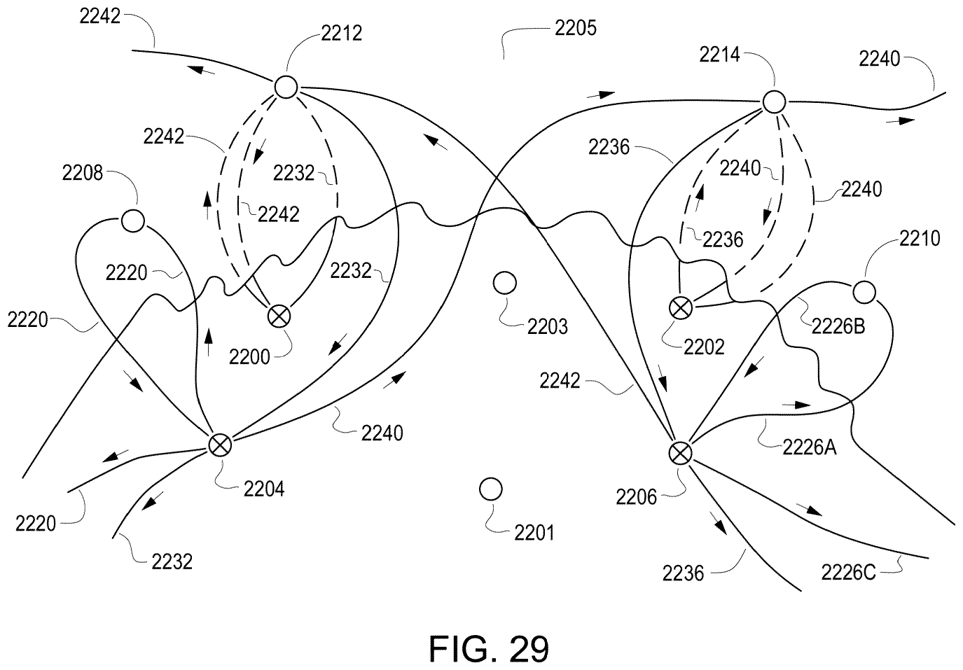

The present disclosure addresses exemplary applications in which doubly armed cinching anchors are advantageous. FIG. 29 is an implementation of the demonstration of one of those applications, specifically, a double row tissue repair. This repair technique is commonly used in rotator cuff repair. The doubly armed anchor can be either the doubly armed hard anchor or the doubly armed soft anchor. The method of this technique allows all knotless fixation to be in a double row configuration. There are four anchors placed, two medial and two lateral. The medial row anchors are single-loaded cinchable anchors, meaning each anchor has a single working suture. Examples of suitable hard anchor versions for this are shown in FIGS. 16, 17, and 18. Examples of suitable soft anchor versions are shown in FIGS. 20, 21, 22, 23, 24, 25, and 26.

In FIG. 29, anchor 2200 and anchor 2202 are the medial row anchors. The lateral row anchors, anchor 2204 and anchor 2206, are doubly armed cinching anchors. Examples of the hard version 1142 of these doubly armed anchors are shown and discussed with regard to FIGS. 27, 27A and 27B. An example soft version of the doubly armed anchor 1242 is shown in FIGS. 28 and 28A.

In the example of a rotator cuff repair, the repair is begun by placing anchor 2200 at the medial aspect of the bone footprint near the articular surface 2203 in the posterior half of the footprint. The working suture 2232 and the shuttles sutures are passed through the rotator cuff at location 2212. Next, anchor 2202 is placed in the anterior half of the footprint adjacent to articular surface 2203. Working suture 2236 and the accompanying shuttle sutures are passed through the rotator cuff at location 2214. Next, doubly armed anchor 2204 is placed in the lateral aspect of the footprint in the posterior half. Working suture 2220 is passed through the edge of the tendon 2205 at location 2208 and then shuttled back through the cinching mechanism of anchor 2204. The free end of suture 2220 is now tensioned to provisionally advance the edge of the tendon out towards the lateral aspect of the footprint. Next doubly armed anchor 2206 is placed in the anterior half at the lateral aspect of the footprint. Working suture 2226 is then passed through the tendon at location 2210. Suture 2226 is then passed back through the cinching mechanism of anchor 2206, and the free end of suture 2226 is then pulled until the tendon edge is advanced out to the lateral edge of the footprint. At this point, the tendon edge has been brought out to its anatomic position. Further fixation is achieved by passing suture 2232 through anchor 2204. Suture 2232 is passed through the cinching mechanism of anchor 204 via the suture shuttles, and the free end of suture 2232 is tensioned to further increase the strength of the repair.

In a like manner, suture 2236 is shuttled through anchor 2206, and once suture 2232 is passed through the cinching mechanism of the anchor, the free end of suture 2236 is then pulled to further increase tension and fixation of the repair. Working suture 2240 is then shuttled through anchor 2202, and once working suture 2240 is passed through the anchor's cinching mechanism, and the free end of working suture 2240 is tensioned to further increase the pressure on the tendon to bone.

Next, suture 2242 (now acting as a crossing/compression suture) is shuttled through anchor 2200, and once suture 2242 has passed through the anchor's cinching mechanism, the free end of 2242 is pulled until adequate tension is achieved. At this point, the tension on all the sutures is still adjustable at the surgeon's discretion. The free ends of sutures 2220, 2232, 2226, 2236, 2242, and 2240 can be further tensioned. In one exemplary implementation, sutures 2220 and 2226. are tensioned first, as this continues to pull the rotator cuff tendon towards its lateral anatomic insertion. Secondarily, tensioning sutures 2232, 2236, 2240, and 2242 compress the tendon to the bone. This is advantageous as tendon compression to bone has been shown to improve healing.

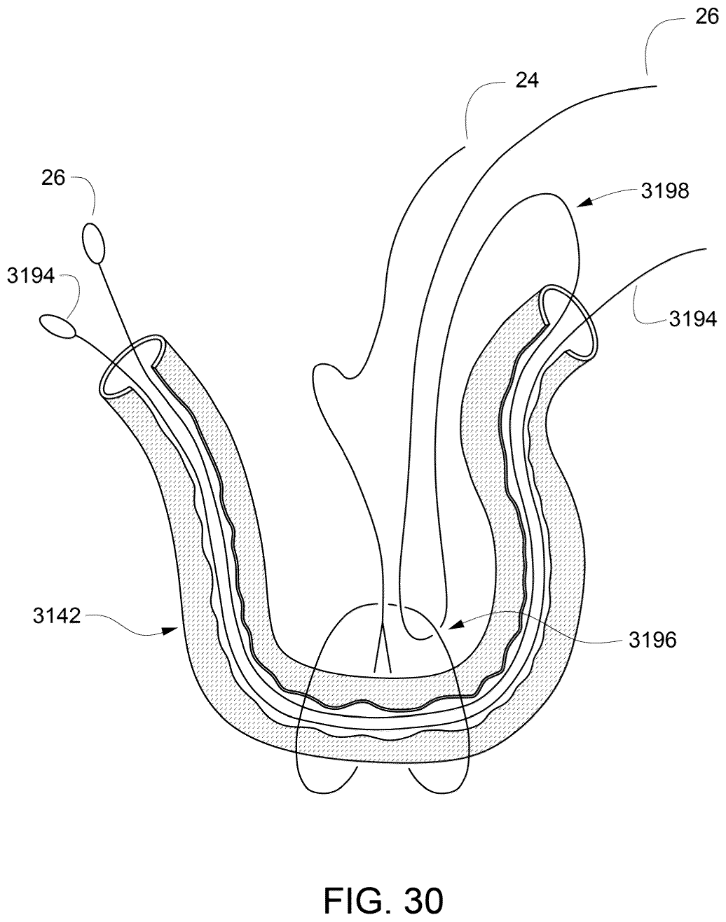

Rotator cuff repair is one example of tissue repair to bone where two rows of suture anchors have been shown to be mechanically superior. FIG. 30 describes a soft anchor 3142 useful for lateral row fixation. Frequently, soft anchors use smaller holes and do less damage to the underlying bone than hard anchors. Thus, it is generally advantageous for a soft anchor to be utilized for the lateral row. The shell of anchor 3142 is constructed of suture and/or woven on nonwoven fabric. A working suture 24 is attached to anchor 3142 by a luggage tag pre-tied knot 96. A shuttle suture 26 shuttles the working suture through the anchor, and an additional, larger shuttle 94 is used to shuttle the medial row sutures through the anchor 1300. The order in which this is completed would be as follows: the anchor 3142 would be deployed into the bone 1146; the medial row anchors are passed through the anchor with an anchor insertion tool (not shown in FIG. 30) still in place until adequate tension is applied to the medial rose sutures. Next, working suture 24 is shuttled through the anchor 3142, after being passed through additional tissue, and then passed through the anchor with shuttle suture 26. As the working suture is tensioned within the anchor 3142, this fixes both the medial row sutures which have been shuttled through the anchor with shuttle 94 and working suture 24.

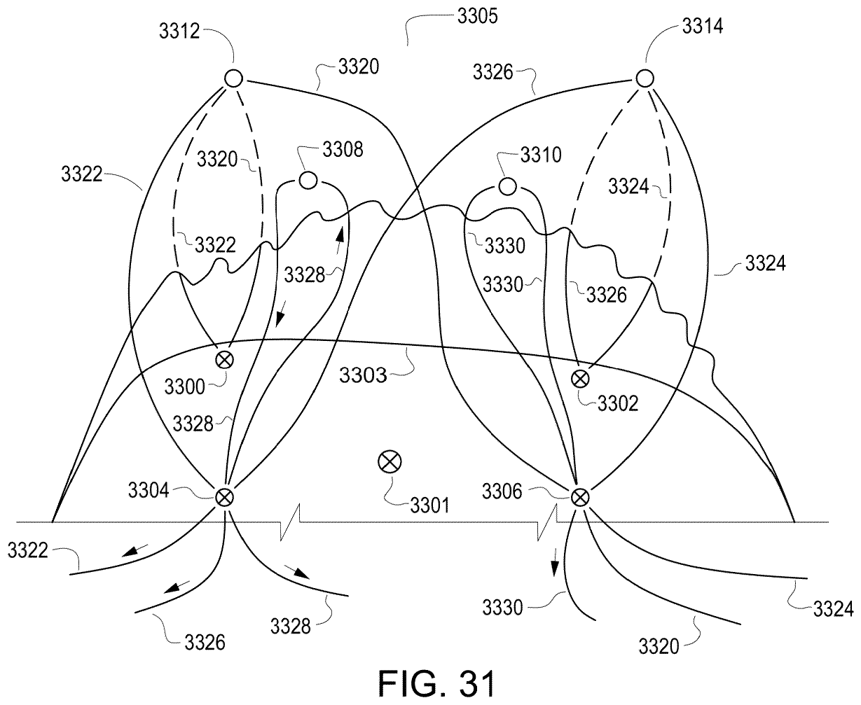

FIG. 31 an exemplary implementation of techniques using the lateral row soft suture described in FIG. 30. In FIG. 31, the lateral row soft anchors 3304 and 3306 are used to form the lateral row anchor fixation for tendon to bone. In a double row repair, the medial row, non-cinching, suture anchors 3300 and 3302 are placed in bone. In FIG. 31, posterior anchor 3300 is shown having sutures 320 and 322 coming from it, and anterior anchor 3302 has sutures 324 and 326 coming from it. Sutures 320 and 322 are passed through the tendon 305 at location 312. Sutures 324 and 326 are passed through tendon 305 at location 314. This leaves four free strands of sutures that have been passed through the tendon. Suture 322 is combined with suture 326 and passed through anchor 3304. The design of anchor 3304 can be substantially the same as or similar to soft suture anchor 3142 described with respect to FIG. 30, which allows passage therethrough of the medial row sutures 322 and 326 prior to insertion into its bone hole. Anchor 3304 is then inserted into its bone hole, and tension is applied to the tails of suture 322 and 326 to advance the tendon 305 out onto the bony surface 301. This provides provisional fixation for the tendon. Next, suture 328, which is the working suture of anchor 3304, is then passed through the edge of the tendon at location 308. Working suture 328 is then shuttled back through anchor 3304 through the internal cinching/locking mechanism, such as described above with respect to soft suture anchor 1142, and tension is applied to the free end of suture 328 until adequate tension is achieved. The internal cinching/locking mechanism for suture 328 also provides additional fixation and friction for sutures 322 and 326 giving a substantially secure fixation resistant to loosening.

Next, the anterolateral anchor 3306 is utilized to fix the remaining sutures. The design of this anchor 3306 can be the same or similar to the soft anchor 3142 shown in FIG. 30. Suture 324 is combined with suture 320 and passed through anchor 306. Anchor 306 is then inserted into its bone hole, and tension is applied to the tails of suture 324 and 320 to advance the tendon 305 out onto the bony surface 301 until it is reduced into the correct position. Next, the working suture 330 in anchor 3306 is passed through the edge of the tendon at location 310 and then shuttled back through the cinching mechanism in anchor 3306. Tension is then applied to the free end of working suture 330 until adequate tension is achieved. The cinching/locking mechanism of anchor 306 also provides additional fixation and friction for sutures 324 and 320, thereby providing a substantially secure fixation resistant to loosening.

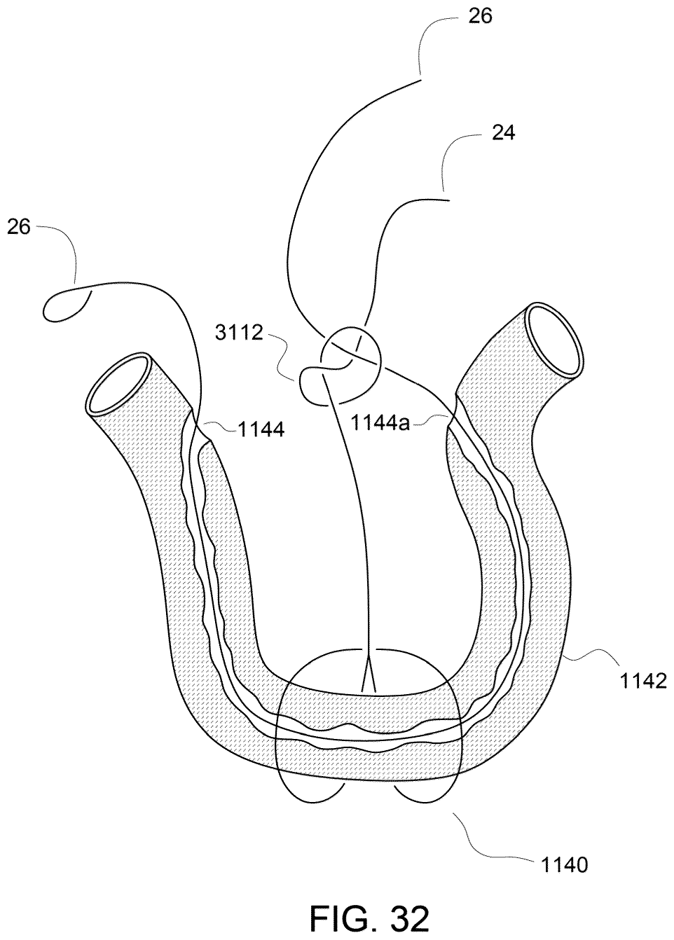

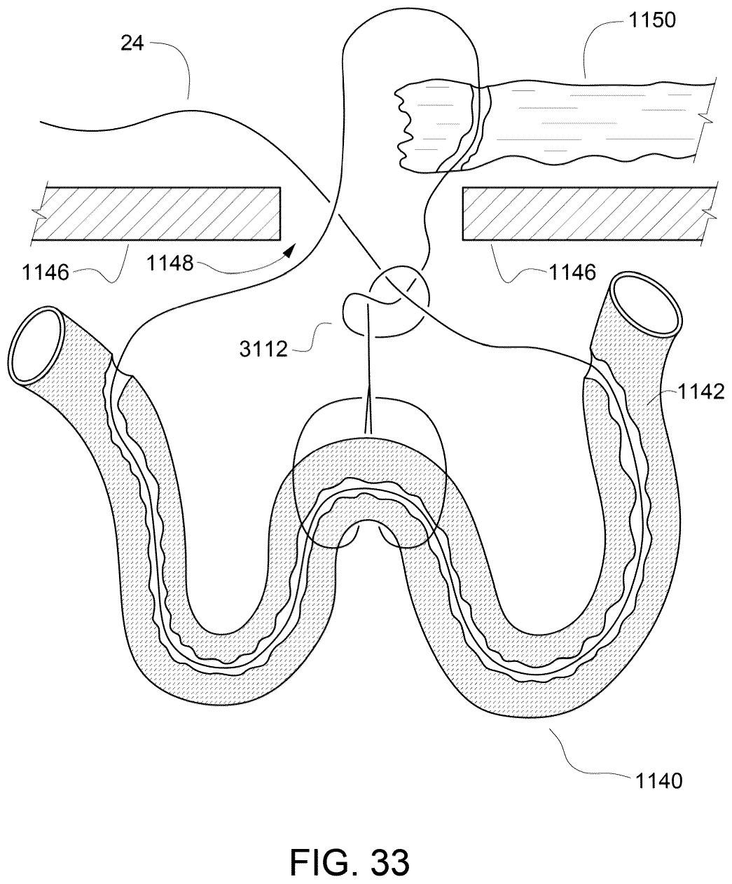

FIG. 32 shows an implementation of soft suture anchor 1142 analogous to the soft suture anchor of FIGS. 20 and 21, except as shown in FIG. 32, a knot 3112 has been added through which shuttle suture 26 passes. Knot 3112 is added to improve symmetry of the anchor 1142 for both insertion and deployment, as shown in FIG. 33. Knot 3112 also adds increased friction for enhancing fixation of the working suture by the cinching/locking mechanism.

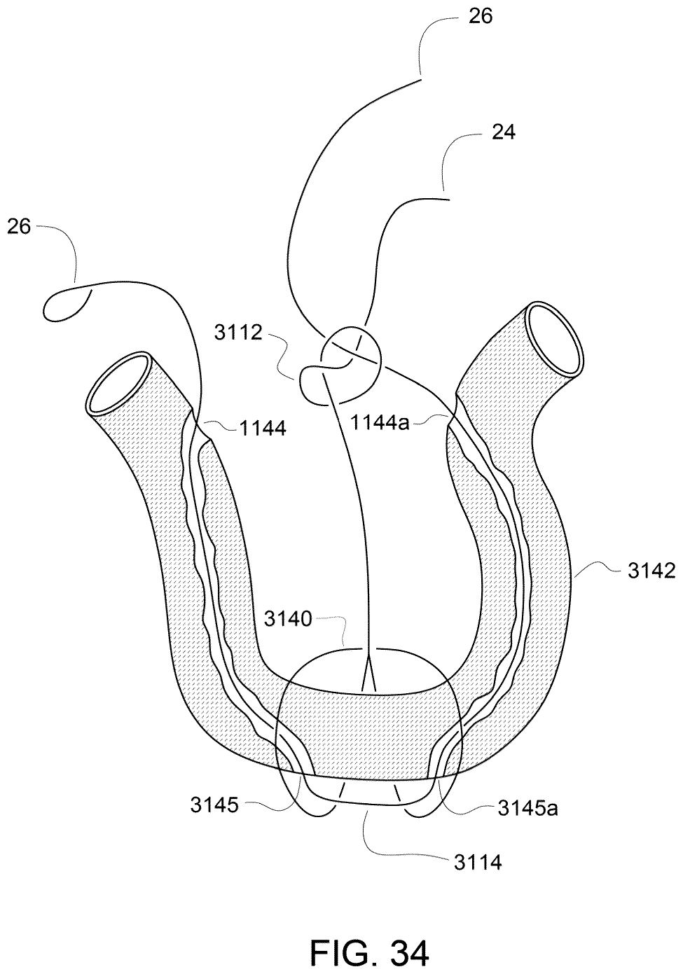

In FIG. 34, such figure is an exemplary implementation of an anchor and suture configuration analogous to that depicted in FIG. 32. However, in FIG. 34, an additional feature includes the shuttle suture 26 penetrating the wall of the anchor 3142 at point 3145 and extending outside of anchor 1142 for a length 3114, and then reentering anchor 1142 at point 3245a, so that shuttle suture 26 contacts the luggage tag knot 3140 directly and more intimately. Luggage tag knot 3140 is similar to the luggage tag 1140 shown as in FIG. 20. The purpose of such luggage tag knots is to increase friction as the suture working suture 24, after working suture 24 is shuttled back through the anchor, passes through the cinching/fixation mechanism.

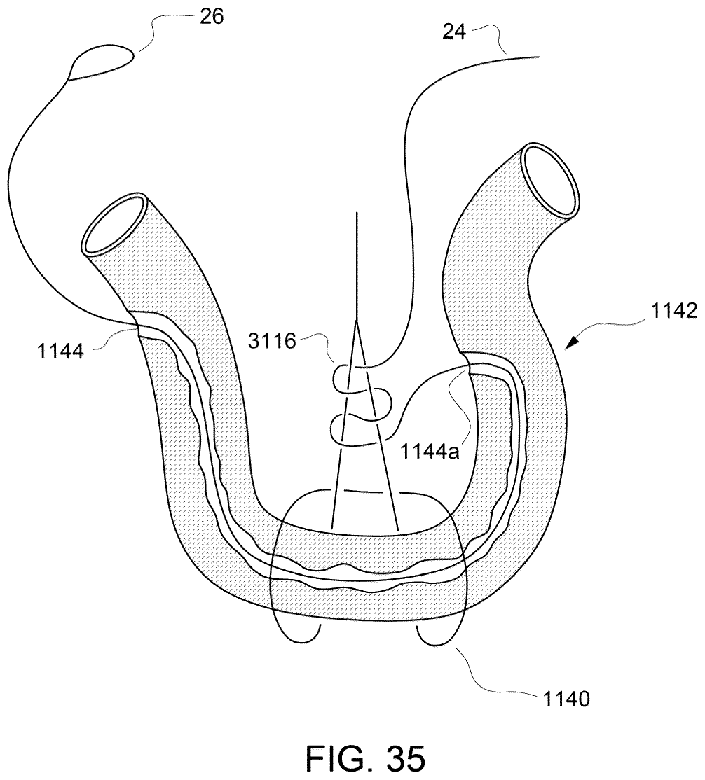

FIG. 35 is another exemplary implementation of an anchor and suture configuration analogous to that depicted in FIG. 20. The modification in this configuration is the interlacing, or weaving, of the shuttle suture 26 through an elongated luggage tag loop 3116. This, once again, improves the symmetry of the anchor 3142 during insertion and increases the friction as the working suture 24 is cinched within the anchor 42. Also, suture 26 entry point 1144 faces away from point 1144a in this implementation.

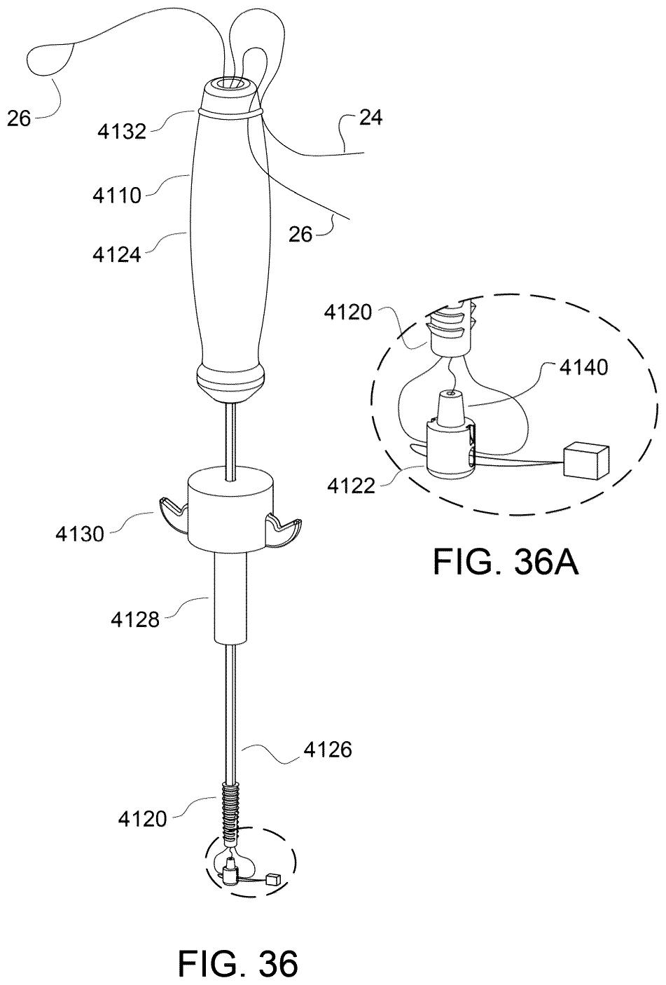

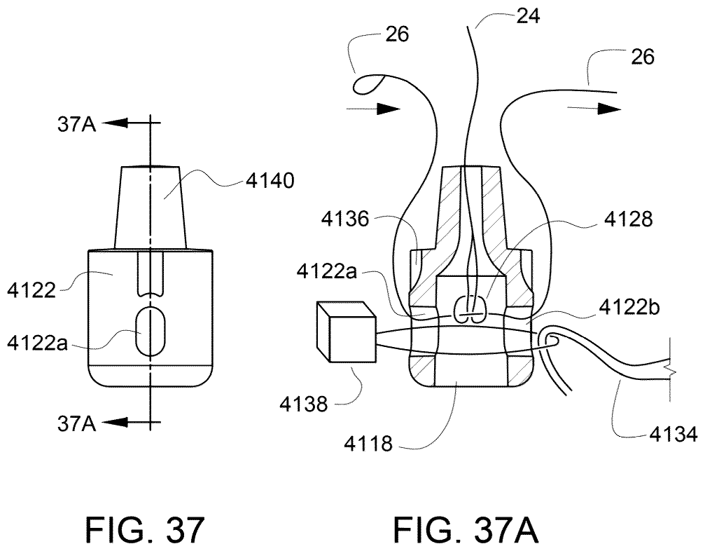

FIGS. 36, 36A, 37, 37A, 38, 38A, and 38B illustrate exemplary implementations of lateral row suture anchor and insertion tool, generally 4110, configured to carry a cinchable working suture 24 and includes a lower eyelet 4122 member for use with a screw in anchor 4120 to lock the medial row anchors via interference fit. Eyelet member 4122 includes essentially the lower third of the hard anchor 1110 discussed above, to form eyelet member 4122, which includes having apertures 4122a and 4122b (FIG. 37A) 4120for receiving suture in a manner similar to the receipt of sutures by fenestrations, slots, or windows, 1131a and 1131b provided in the lower portion of anchor 1110. Eyelet member 4122 fits on the tip of the screw-in lateral row anchor 4120.

FIGS. 36 and 36A represent an exemplary implementation of a design for a lateral row anchor application. A hole would be predrilled or tapped in the desired bony location. The tip of this device including the eyelet member 4122 and a portion of the hexagonal (or other desired cross-sectional shape) shaft 4126 of insertion tool 4110 would be inserted into the hole to a desired depth. Prior to insertion of the eyelet, the medial row sutures are loaded through the suture loading mechanism 4138 at the bottom of the anchor. Insertion tool 4110 is then rotated, using a handle 4124, which allows the threads of a screw-in portion of the anchor 4120 to engage the bone. Handle 4124 is rotated until suture anchor 4120 is entirely within the bone. This screw-in portion of the anchor 4120 fixes the medial row anchors via interference fit between the screw and the bone.

Other features of insertion tool 4110 include cleats 4130, which allow the medial row sutures to be tensioned and cleated prior to screwing in the anchor 4120. A pusher tube 4128 includes a sleeve 4128a received on a shaft 4126 extending from handle 4124 of insertion tool 4110. Use of insertion tool 4110 allows the surgeon to keep continuous downward pressure on the screw-in anchor 4120 to encourage engagement with the bone. The pusher tube 4128 can be equipped with reverse threaded screw threads to prevent it from migrating.

Once screw-in anchor 4120 is fully engaged, an O-ring 4132 carried on and encircling the handle 4124, proximate the extreme end of handle 4124, can be removed, which releases the preloaded sutures 24 and 26.

Once screw 4120 is fully seated, and the sutures are released, the insertion tool 4110 can be removed. This leaves the eyelet 4122, the screw-in, or, interference, suture anchor 4120, and the sutures 24 and 26 implanted in the bone. Also implanted at this point are the medial row sutures, which can then be cut.

FIGS. 37 and 37A provide further detail of an exemplary implementation of the eyelet member 4122 portion of the anchor. To use the eyelet member 4122 for a double row repair, the first step is to load medial row sutures 4134 into the eyelet. This is accomplished by threading the sutures 4134 through the suture slots, passages, or, windows 4122a and 4122b with the suture loader 4138. The suture loader 4138 is threading device that operates in in some respects like a needle threading device used for threading a sewing needle. Once the medial row sutures 4134 are pulled through the anchor, they may be placed into a pre-drilled hole and tensioned as described in FIG. 36. The cinching mechanism within this eyelet is similar to that shown in FIGS. 13, 13A, 14, 14A. A cavity 18 in the bottom portion of the anchor is shaped and/or may include surface texture features to increase friction relative to the sutures. A luggage tag pre tied knot 4128 is provided through which a shuttle suture 26 passes. Working suture 24 is routed through eyelet member 4122 in substantially the same manner as working suture shown in FIGS. 13A, 14, and 14A.

Once the medial sutures have been tensioned and the screwdriver type portion of the insertion tool 4110 has been removed, eyelet member 4122 functions as a cinchable knotless suture anchor. After the medial row anchors are tensioned, there is often a need for additional fixation of the edge of the soft tissue. In this instance, working suture 24 can be passed through the additional soft tissue and then shuttled back through the anchor eyelet with its internal cinching mechanism. Suture 24 is pulled and tensioned by the surgeon until the desired level of fixation has been obtained. The eyelet allows unidirectional passage, so once suture 24 is tensioned, it is restrained from loosening.

Other features include the hexagonal shaft 4126 of the insertion tool 4110 being cannulated, which allows sutures 24 and 26 to pass therethrough. The eyelet member 4122 is, in an exemplary implementation, shaped so that a nipple-shaped upper portion 4140 of the eyelet fits inside the hexagonal shaft 4126. The eyelet is held onto the tip of the shaft by tension in sutures 24 and 26. Release of the eyelet from the tip of the anchor simply requires loosening the sutures 24 and 26 by removing the O-ring 4132. The shaft is then pulled backwards out of the hole to remove the shaft 4126 from the eyelet member 4122. Slots 4136 on either side of the eyelet allow smooth passage of the sutures around the eyelet and up into shaft 126.

FIGS. 38 and 38A illustrate further detail from FIGS. 36, 36A, 37, and 37A, including illustration of the passage of the tails of the medial row sutures through the eyelet member 4122 via suture windows 4122a and 4122b. FIG. 38 is an exemplary implementation of the anchor 4110 after it has been inserted into bone and shows medial row sutures 134 which originated from medial anchor 4160. Anchor 4160 can be any commonly used suture anchor which has free sutures which may be passed thru tissue. Medial row sutures 134 pass through the soft tissue at location 4166. The sutures then pass over the tissue and are then fixed at a second point with anchor 4120. As anchor 4120 is screwed into the bone via the anchor shaft 4126, the sutures 134 are fixed via an interference fit between the screw 4120 and the wall of the bone hole 162. Once the media row of sutures 134 have been fixed, the free tails may be cut and discarded. After the screw anchor 4120 has been inserted to an appropriate depth, insertion tool 4110, which includes shaft 4126 is removed. O-ring 4132 is released which will allow the handle 4124 to fully removed. This will leave working suture 24 and the two ends of the shuttle suture 26 still attached to the anchor 4110 configuration. These sutures can subsequently be used to attached additional tissue to the anchor in a manner similar to that shown in FIGS. 17 and 18.

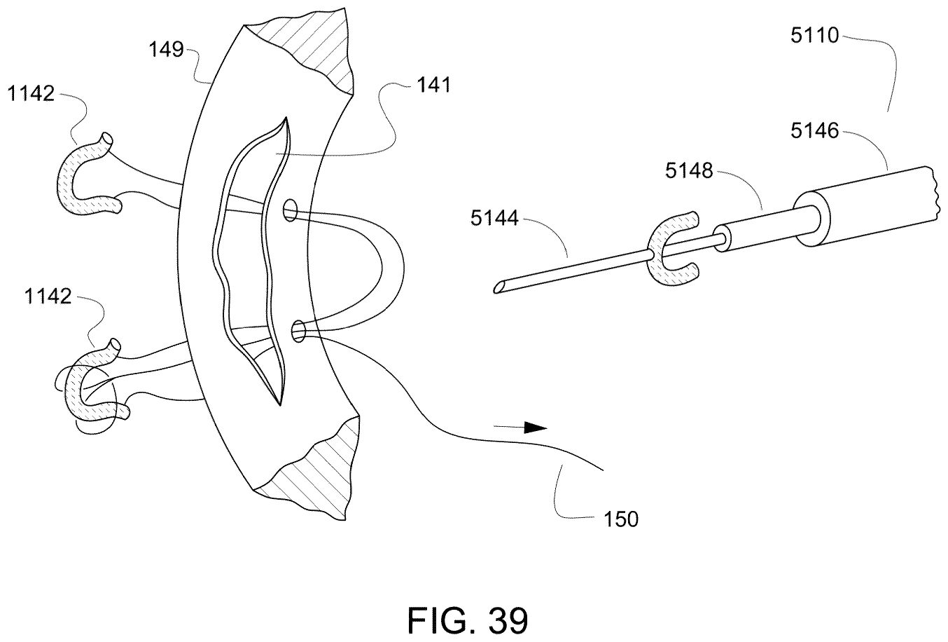

FIG. 39 illustrates an exemplary implementation of an assembly, generally 5110, for use with one or more of meniscal repair devices disclosed herein, which utilizes the same cinching mechanism within the soft anchors discussed above, such as in regard to that shown in FIGS. 20 and 21. Meniscal repair devices must compress the torn tissue together to promote healing. Also, during installation of the suturing device, damage as to the meniscus must be minimized. To also promote healing and avoid iatrogenic injury, it is important to maintain a very low diameter of the suturing device as it is passed through the meniscus tissue. If the diameter is too large it could cause permanent damage to the tissue, with poor long-term results. Additionally, because of the difficulty in working arthroscopically in a small space, targeting of device insertion during surgery is difficult working arthroscopically. Meniscal repair devices have been known to be fragile and to suffer from an inability to precisely place the device where it will be most effective.

The features of meniscal repair device assembly 5110 which enhance targeting ability include a rigid single or double-barrel tube 5146 and a low diameter piercing needle 5144. Also, device 5110 is equipped with a plunger 5148, which allows the meniscal suture anchors to be passed through a very low diameter hole. The velocity of the plunger 5148 can be increased to the point that it allows insertion of the anchor with minimal tissue disruption. An analogous biologic scenario could include where shrapnel passing through skin at a high velocity creates a very small entrance wound compared to the size of the shrapnel. Analogously, high speed passage of the meniscal anchor produces a very small entrance wound through the meniscus and thus limits damage to the tissue during insertion. The velocity of plunger 5148 can be controlled by a spring-loaded or gun type mechanism, or simply inserted by a handle. meniscal repair device 5110 can be used either during arthroscopic or open surgery.

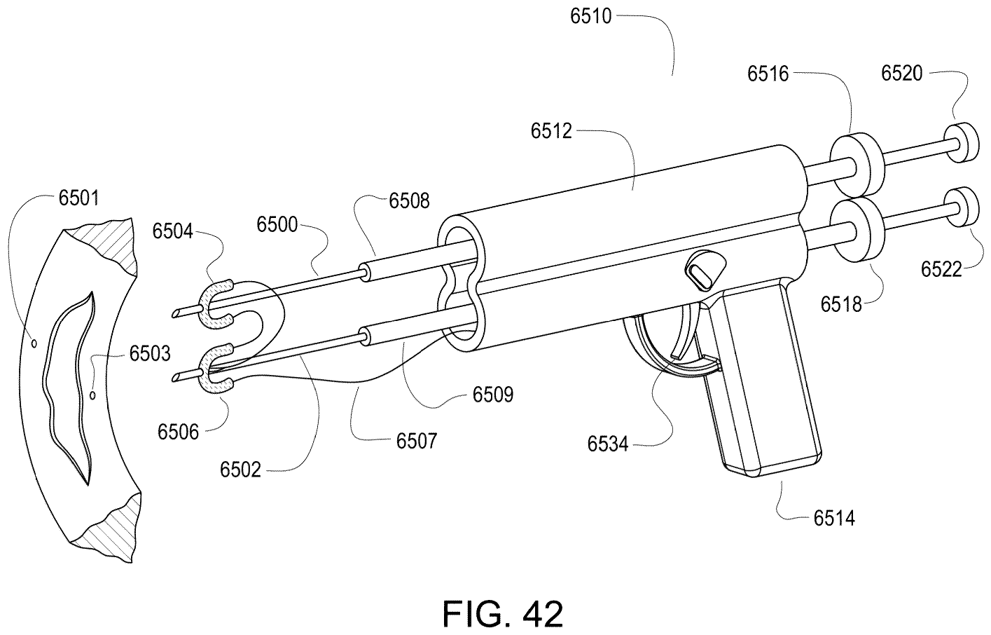

Prior to implantation, one or more suture anchors 1142 are loaded onto the piercing needle 5144. (It should be noted that there can be two piercing needles in a double barrel tube 5146, as shown in FIGS. 42-45.) The first step is to pass piercing needle 5144 through the meniscus tear 141. For safety, this is passed to a pre-prescribed controllable depth. The insertion device is equipped with a stop mechanism (not shown) to prevent insertion to an unsafe depth. The anchor must be passed to the periphery of the meniscus 149. The anchor is pushed through to the periphery 149 by inserting plunger 5148 to the appropriate depth. The piercing needle 5144 is backed out of the tear and moved to an adjacent location and then passes back through the meniscus. Then anchor 1142 is passed through the meniscus tear to the periphery 149, once again by using plunger 5148 to pass it to the appropriate depth. The delivery device is now removed leaving anchors 1142. To compress the meniscus tear, the surgeon pulls on suture 150 until adequate tension in suture 150 is obtained. The internal locking/cinching mechanism within suture anchor 1142, such as discussed above regarding soft suture anchors disclosed herein, allows unidirectional passage of the suture, and ultimately cinches it. The more force applied to suture 150, the tighter the repair becomes. Once the desired compression is obtained, suture 150 is cut flush with the surface of the meniscus, leaving no exposed knots and a high-quality high-tension repair.

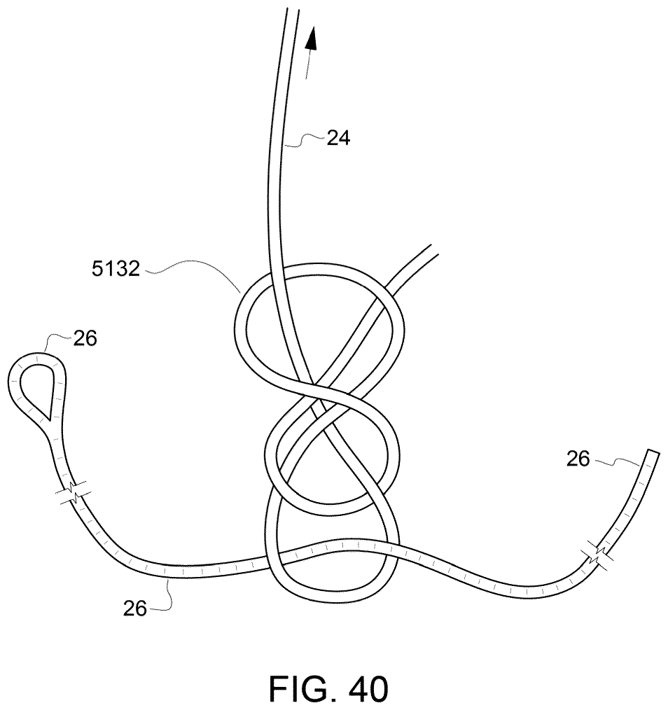

In FIG. 40, an exemplary implementation of pretied knot 5132 is shown schematically. Knot 5132 is designed to create high levels of friction between the two sutures. In deployment of cinchable anchors, such as anchors 1110 and 1142, the tail of the working suture 24 is pulled back through the pre-tied knot 5132 by shuttle suture 26. This creates a loop of repair suture through the tissue as shown in previous figures. Then the surgeon pulls on the working suture 24, and the more that suture 24 is tensioned, the tighter the pre-tied knot 5132 becomes, thus substantially restraining knot 5132 from loosening and giving sufficient compression of the tissue. Knot 5132 creates high levels of friction on its own, but the combination of the friction of the pre-tied knot and the conically shaped (or other uniquely shaped) bottom of the suture anchor 42 (in the case of hard suture anchor) creates a relatively high level of friction to restrain loosening of the suture. This is just one example of a pre-tied knot, and many other configurations can be used.

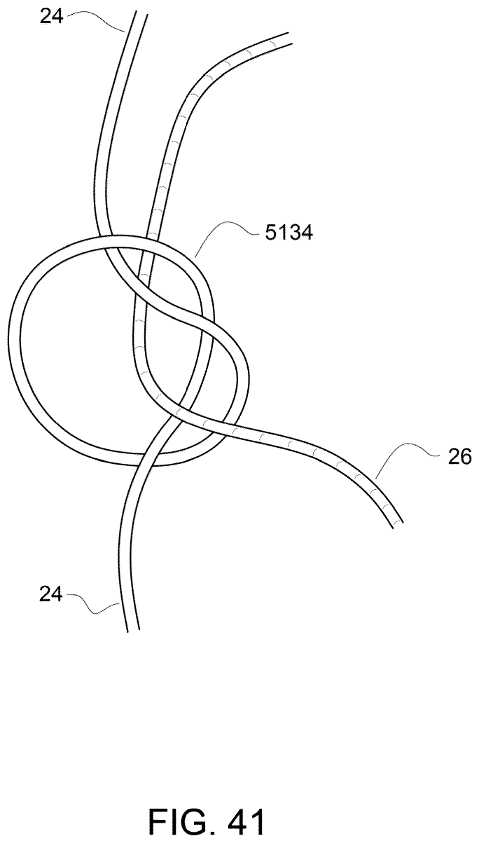

FIG. 41 shows an exemplary implementation of a simple knot 5134 through which suture 24 passes to increase friction. Knot 5134 corresponds to knot 5132 shown in FIG. 19 and can be a simple knot created in the working suture 24, with the shuttle suture 26 passing through knot 4134.

FIG. 42 shows an exemplary implementation of an insertion tool, generally 6510, for use in the meniscal repair system discussed above and in relation to FIG. 39. To create a repair with this anchor system, two points of fixation are provided. In FIG. 42, insertion tool is shown as a double barrel configuration. Insertion tool 6510 is shaped like a gun with a handle 6514 which allows the surgeon to comfortably hold the device while positioning the implants. Insertion tool 6510 could also be made with a single barrel gun with an internal switching mechanism to shift between deploying the first needle 6504 and the second needle 6506 into the barrel of the gun. Insertion tool 6510 is pre-loaded with low diameter needles 6500 and 6502. Suture anchor 6504 is preloaded as well, by sliding it on to needle 6500 in a manner not unlike how a skewer would be used for food. In like manner, anchor 6504 is loaded onto needle 6502. Suture anchor 6504 can be identical to the anchor shown in FIG. 20. For the purposes of meniscal repair, anchor 6504 can be miniaturized to create the correct performance profile for meniscus repair. Suture anchor 6504 is like the anchor shown in FIG. 20, but there is no luggage tag know cinching mechanism, as the suture passes freely through anchor 6504. Both anchors 6504 and 6506, after they have passed through the meniscus periphery, expand, and are thus restrained from pulling out.

Pusher tubes number 6508 and 6509 of insertion tool 6510 are pre-assembled. These have an inner diameter which accommodates needles 6500 and 6502. The barrel of the gun type insertion tool 6510 assembly is shown as 6512. The inner diameter of the barrel is just large enough to accommodate all the aforementioned parts.

In the example shown in FIG. 42, insertion tool 6510 is shown as being double barreled. The design of barrel (or barrels) 6512 can be modified in numerous configurations, and one or more additional barrels could be provided if desired. The length of the barrel(s) is long enough to accommodate elements 6500 through 6509 discussed above. The outer size of the barrel(s) configuration 6512 is kept small enough to allow easy passage into the knee during arthroscopy. The barrel(s) can be shaped in numerous curves to allow easy passage to different parts of the meniscus. The barrel(s) can also be rotated with respect to the handle to make it more comfortable for the surgeon to insert the device.

To perform a meniscal repair, needle 6500 is passed through the meniscus, at point 6501, to a predetermined depth. Handle 6516 is pushed inward to force plunger 508 to insert anchor 6504 into the meniscus. Once anchor 6504 has passed the periphery and deployed, needle 6500 can be completely withdrawn, by pulling on handle 6520, leaving just the anchor implanted. These steps are repeated at a different location, in this case at point 6503, which is chosen to optimally compress the tear. Needle 6502 is passed through the meniscus at this new location 6503 and plunger 6509 is pushed by handle 6518 through the meniscus until anchor 6506 has passed through the periphery. Needle number 6502 is fully withdrawn by pulling on handle 6522 and the entire assembly is withdrawn. To cinch the anchors and complete the repair, the surgeon simply pulls on suture 6507 until adequate tension is created. The cinching mechanism an anchor 6506, which only allows uni-directional passage of this suture, fixes the suture with respect to the anchor, i.e., the tightening of the suture by the surgeons creates increasing tension and compression of the tear while restraining loosening. This process can be repeated as many times as necessary, based on the surgeon's discretion, until the desired repair is obtained.

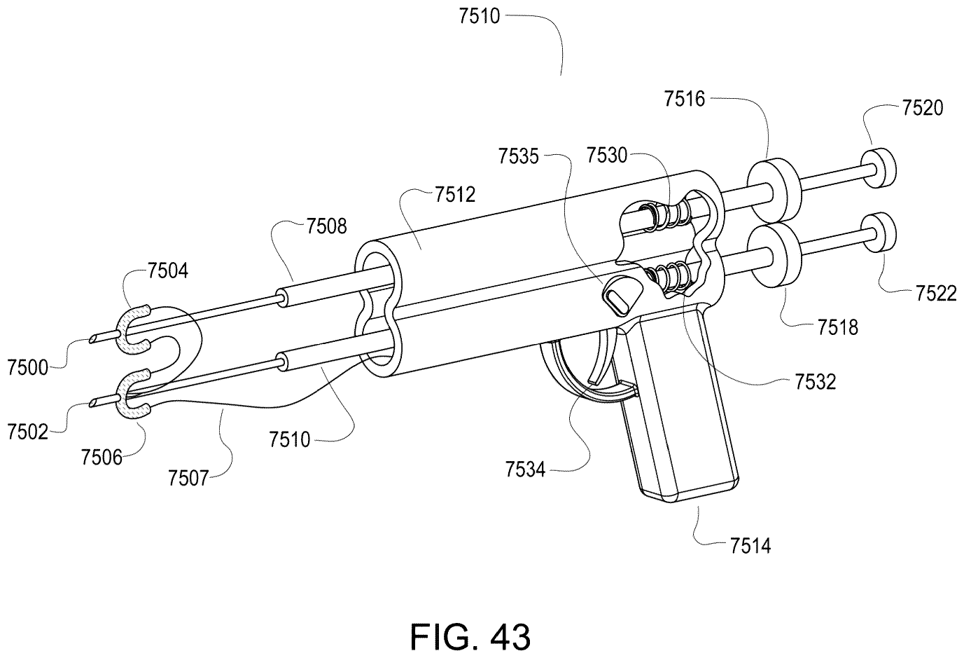

FIG. 43 shows an exemplary implementation of an insertion tool, generally 7510, like the insertion tool 6510 shown in FIG. 42, for manual insertion of the suture anchors. Insertion tool 7510 is a spring-loaded device designed to create a higher velocity in advancing the pusher tubes 7508 and 7509. This increased velocity allows the suture anchors 7504 and 7506 to be forced through the meniscus more quickly. This feature should make the device easier to use for the surgeon and should result in better control and less damage to the meniscus. Insertion tool 7510 comes preloaded with the components shown in FIG. 43.

To start the process, insertion tool 7510 is inserted into the joint, and needle 7500 is inserted into the meniscus to approach prescribed depth. Handle 7516 is pulled rearwardly and “cocked” in a manner similar to how one would cock a gun. The mechanism 7530 is a spring-loaded mechanism which allows cocking and releasing of the mechanism. When the surgeon is ready, trigger 7534 is utilized to fire the insertion tool 7510, which causes plunger 7508 to rapidly advance and force anchor 7504 through the meniscus into appropriate position. In one implementation, insertion tool 7510 is equipped with a toggle switch 7535 which allows this procedure to be repeated with the other anchor. After the toggle switch 7535 is flipped to the alternate position, needle 7502 is passed through the meniscus using handle 7522, and handle 7518 is used to cock the insertion tool 7510. Mechanism 7532 includes a spring-loaded mechanism generally the same as 7530. Once the surgeon is ready, the surgeon pulls trigger 7534, which causes a plunger 7509 to advance and force at high velocity anchor 6506 through the meniscus. Both suture anchors now being deployed, the insertion tool 7510 can be removed from the joint. Next, the surgeon simply pulls on suture 7507 until adequate tension is obtained across the repair. A conventional knot pusher (not shown) may be used at this point to create better leverage for tensioning if necessary. Once adequate tension is obtained, the surgeon cuts suture 7507 flush with the surface of the meniscus, leaving no exposed knots.

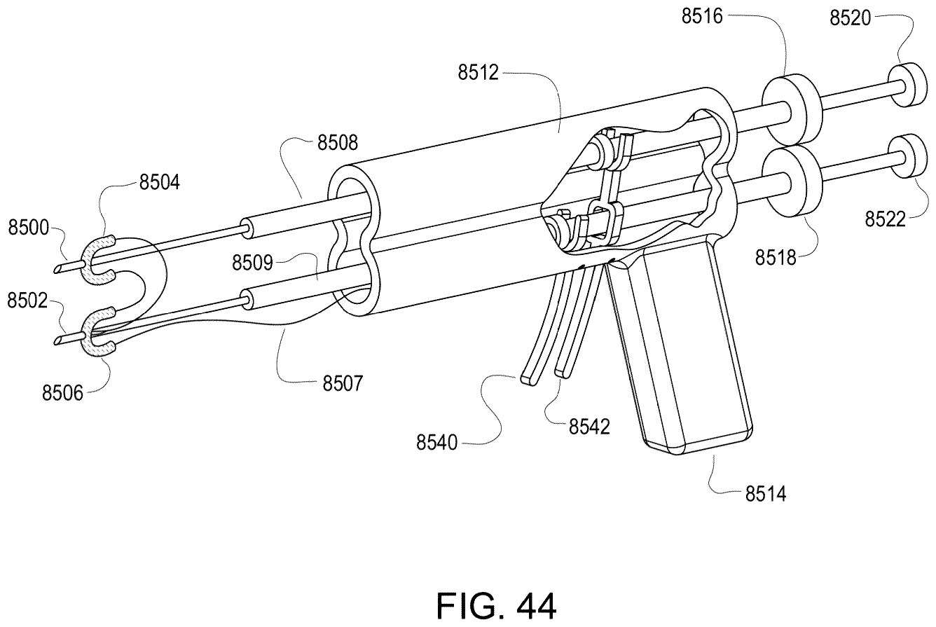

FIG. 44 illustrates another exemplary implementation of an insertion tool, generally 8510, similar to that shown in FIG. 43. Insertion tool 8510 is used in generally the same manner as insertion tool 7510 to repair a meniscus tear. The primary difference between the two devices is that this is equipped with lever 8540 and lever 8542. Insertion tool 8510 includes at least one mechanism similar to a caulk gun. In use, a lever 8540 is squeezed to advance a plunger 8509 and forces anchor 8506 down needle 8502 and into the meniscus. Subsequently, lever 8542 is squeezed to advance plunger 8508 to force anchor 8504 down needle 8500 and through the meniscus. Once the anchors 8500 and 8506 are placed in the meniscus, the insertion tool 8510 is withdrawn, and the surgeon pulls on suture 8507 until adequate tension of the repair has been obtained.

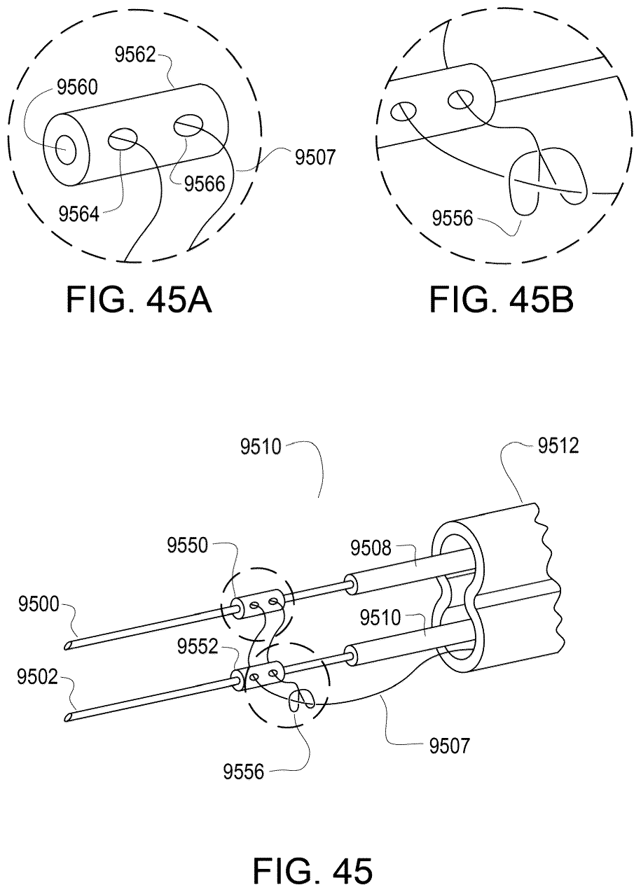

In FIGS. 45 and 45A, another exemplary implementation of a meniscus repair assembly, generally 9510, is shown, which is similar to counterpart assemblies on the insertion tool 7510 shown in FIGS. 42 and 43, and insertion tool 8510 shown in FIG. 44, and is for use in connection with one or more of such insertion tools. The primary difference between assembly 9510 shown in FIG. 45 compared with the preceding insertion tools is that the implants are hard suture anchor implants, typically made of PEEK or bio-composite polymer, rather than soft suture anchors. These implants are inserted with essentially the same technology utilized in the devices shown in FIGS. 39, 42, 43, and 44. The devices which can be utilized to insert hard suture anchors 9550 and 9552 are essentially identical to those devices pictured in FIGS. 42, 43, and 44. The procedure is essentially identical as well. Needles 9500 and 9502 are utilized to pierce the meniscus in the desired location. Plunger 9508 is utilized to force anchor 9550 through the meniscus. Then, plunger 9509 is used to force anchor 9552 through the meniscus. The insertion tool 9510 can then be removed, and the repair can be cinched down. Cinching is accomplished by pulling on suture 9507 until the desired compression is achieved. The pre-tied knot 9554, which can be a luggage tag pre-tied knot, allows the device to cinch down but not loosen. A luggage tag configuration is shown as 9556 in FIGS. 45 and 45B.

In FIG. 45A, an exemplary implementation of how the suture passes through the hard implants 9550 and 9552 is shown. The inner diameter of the implant 9560 is just large enough to accommodate the needles 9500 or 9502. The outer diameter 9562 is a low diameter compared to most meniscal repair devices, so that minimal damage is done to the meniscus with insertion of the implant. The implant is equipped with holes 9564 and 9566 to allow for passage of suture through the implant. Suture 507 can be of various diameters and materials. Alternatively, insertion tool assembly 9510 can be equipped with a looped end to form a luggage tag 9556, as shown in FIGS. 45 and 45B. Luggage tag 9556 could also be a pretied knot.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” /d/ or “comprising,” when used in this specification, specify the presence of stated features, elements, and/or components, but do not preclude the presence or addition of one or more other features, steps, operations, elements, components, and/or groups thereof.

What is claimed is:

1. An anchor for implantation in tissue, the anchor comprising:
a shell defining at least one passage;
a first suture in the at least one passage attached to the shell;
a longitudinally extending suture entwinement in the at least one passage; and
a shuttle suture having a first end and a second end; the first end being configured to engage the first suture,
wherein:
the shuttle suture passes in a first direction in the at least one passage and loops to pass in a second direction generally opposite to the first direction into and through the longitudinally extending suture entwinement, and
upon the first suture being received by the first end of the shuttle suture and the second end of the shuttle suture being pulled:
the first suture passes into the longitudinally extending suture entwinement; and
the longitudinally extending suture entwinement locks the first suture to the anchor.

2. The anchor of claim 1, wherein:
the at least one passage comprises a first passage and a second passage,
the longitudinally extending suture entwinement is proximate to the second passage, and
the shuttle suture passes in the first passage and loops to pass into the second passage in the second direction into and through the longitudinally extending suture entwinement.

3. The anchor of claim 1, wherein the longitudinally extending suture entwinement is in a generally double helix formation comprising the shuttle suture and a first portion of the first suture and is configured to allow the shuttle suture to move longitudinally in only one direction with respect the first portion of the first suture.

4. The anchor of claim 1, wherein the longitudinally extending suture entwinement comprises the shuttle suture and a first portion of the first suture being generally interlaced or coiled with one another.

5. The anchor of claim 1, further comprising an entwinement suture fixed to the shell, wherein the longitudinally extending suture entwinement:
is in a generally double helix formation comprising the shuttle suture and the entwinement suture: and
is configured to allow the shuttle suture and the first suture to move longitudinally in only one direction with respect the entwinement suture.

6. The anchor of claim 1, wherein the longitudinally extending suture entwinement comprises the shuttle suture and an entwinement suture being generally interlaced or coiled with one another.

7. The anchor of claim 2, further comprising a partition defining the first passage and the second passage,
wherein:
the partition defines a partition passage and a post,
the shuttle suture passes through the partition passage when the shuttle suture passes from the first passage to the second passage, and
the first suture is tied to the post to attach the first suture to the shell.

8. The anchor of claim 7, wherein:
the shell defines at least one lateral passage in communication with at least one of the first passage, the second passage, and the partition passage, and
the at least one lateral passage is configured to allow access to the first suture sufficient for attaching the first suture to the post.

9. An anchor for implantation in tissue, the anchor comprising:
an elongated shell defining at least one first passage;
a partition defining at least one second passage generally perpendicularly intersecting the at least one first passage and in communication with the exterior of the shell;
a first suture in the at least one first passage attached to the partition;
a unidirectional suture locking mechanism in the at least one first passage; and
a shuttle suture having a first end and a second end; the first end being configured to engage the first suture,
wherein:
the shuttle suture passes in a first direction in the at least one passage and loops to pass in a second direction generally opposite to the first direction into and through the unidirectional suture locking mechanism, and
upon the first suture being received by the first end of the shuttle suture and the second end of the shuttle suture being pulled, the first suture passes into the unidirectional suture locking mechanism, and the unidirectional suture locking mechanism locks the first suture to the anchor.

10. The anchor of claim 9, wherein the unidirectional suture locking mechanism comprises a woven sleeve.

11. The anchor of claim 9, wherein the unidirectional suture locking mechanism comprises a finger trap.

12. The anchor of claim 9, wherein the unidirectional suture locking mechanism comprises a noose.

13. The anchor of claim 9, wherein the unidirectional suture locking mechanism comprises a pre-tied knot.

14. The anchor of claim 9, wherein the unidirectional suture locking mechanism comprise a suture entwinement comprising the shuttle suture and at least one of the first suture and an entwinement suture.

15. The anchor of claim 9, wherein
the shell comprises a first end in communication with at least one of the first passage and the second passage and a second end opposite the first end, and
the anchor further comprises a body member attached to the second end and defining a suture passage configured for receiving at least one of the first suture and the shuttle suture.

16. The anchor of claim 15, wherein the body member is configured to pivot with respect to the shell.

17. An anchor for implantation in tissue, the anchor comprising:
an elongated woven shell defining a passage;
a first suture having a first end and a second end,
wherein:
the first suture passes through the passage along the length of the elongated woven shell, and
the first end is configured to engage with the second end in a manner that attaches the first suture to the elongated woven shell;
a unidirectional suture locking mechanism in the passage connected to the first suture; and
a shuttle suture having a first end and a second end,
wherein:
the first end is configured to engage the first suture,
the shuttle suture passes in a first direction in the at least one passage and into and through the unidirectional suture locking mechanism, and
upon the first suture being received by the first end of the shuttle suture and the second end of the shuttle suture being pulled, the first suture passes into the unidirectional suture locking mechanism, and the unidirectional suture locking mechanism locks the first suture to the anchor.

18. The anchor of claim 17, wherein:
the elongated woven shell is generally U-shaped, and
the shuttle suture passes in a first direction in the at least one passage and loops to pass in a second direction generally opposite to the first direction into and through the unidirectional suture locking mechanism.

19. The anchor of claim 17, wherein:
the first end of the first suture comprises a loop, and
the second end of the first suture passes through the loop in a manner that attaches the first suture to the elongated woven shell.

20. The anchor of claim 17, wherein the unidirectional suture locking mechanism comprises at least one of a group consisting of a woven sleeve, an elongated suture entwinement, a finger trap type device, a pre-tied knot, and a hangman noose.
